(19) United States
(12) Patent Application Publication
(10) Pub. No.: US 2026/0308163 A1
(43) Pub. Date: Oct. 8, 2026

(54) CRYSTALLINE FORMS OF A PARP1 INHIBITOR

(71) Applicant: Gilead Sciences, Inc., Foster City, CA (US)

(72) Inventors: Jing Liu, San Diego, CA (US); Jayachandra P. Reddy, West Palm Beach, FL (US); Stefani Kocevska, San Francisco, CA (US)

(21) Appl. No.: 19/428,821

(22) Filed: Dec. 22, 2025

Related U.S. Application Data
(60) Provisional application No. 63/738,612, filed on Dec. 24, 2024.

(51) Int. Cl. C07D 491/048; A61K 31/496

(52) U.S. Cl. CPC C07D 491/048; A61K 31/496

(57) ABSTRACT

The present disclosure relates to salts and solid forms of a compound that are inhibitors of PARP and their uses as therapeutic agents for treating diseases, such as cancer.

### CROSS-REFERENCE TO RELATED APPLICATIONS

This application claims the benefit under 35 U.S.C. § 119(e) of U.S. Provisional Application No. 63/738,612, filed on Dec. 24, 2024, which is hereby incorporated herein by reference in its entirety for all purposes.

### FIELD

The present disclosure relates to salts and solid forms of a compound that inhibit PARP and their uses as therapeutic agents for treating diseases, such as cancer.

### BACKGROUND

Poly(ADP-ribose)polymerase (PARP) or poly(ADP-ribose)synthase (PARS) has an essential role in facilitating DNA repair, controlling RNA transcription, mediating cell death, and regulating immune response. These actions make PARP inhibitors targets for a broad spectrum of disorders. PARP inhibitors have demonstrated efficacy in numerous models of disease, particularly in models of ischemia reperfusion injury, inflammatory disease, degenerative diseases, protection from adverse effects of cytotoxic compounds, and the potentiation of cytotoxic cancer therapy. PARP has also been indicated in retroviral infection and thus inhibitors may have use in antiretroviral therapy. PARP inhibitors have been efficacious in preventing ischemia reperfusion injury in models of myocardial infarction, stroke, other neural trauma, organ transplantation, as well as reperfusion of the eye, kidney, gut, and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes (as well as complications) and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing cell death of cancer cells, limiting tumor growth, decreasing metastasis, and prolonging the survival of tumor-bearing animals.

PARP1 and PARP2 are the most extensively studied PARPs for their role in DNA damage repair. PARP1 is activated by DNA damage breaks and functions to catalyze the addition of poly (ADP-ribose) (PAR) chains to target proteins. This post-translational modification, known as PARylation, mediates the recruitment of additional DNA repair factors to DNA lesions.

Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy.

Inhibition of PARP family enzymes has been exploited as a strategy to selectively kill cancer cells by inactivating complementary DNA repair pathways. A number of pre-clinical and clinical studies have demonstrated that tumor cells bearing deleterious alterations of BRCA1 or BRCA2, key tumor suppressor proteins involved in double-strand DNA break (DSB) repair by homologous recombination (HR), are selectively sensitive to small molecule inhibitors of the PARP family of DNA repair enzymes. Such tumors have deficient homologous recombination repair (HRR) pathways and are dependent on PARP enzymes function for survival. Although PARP inhibitor therapy has predominantly targeted SRCA-mutated cancers, PARP inhibitors have been tested clinically in non-SRCA-mutant tumors, those which exhibit homologous recombination deficiency (HRD).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double strand breaks (DSBs) through collapse of replication forks in S-phase. It is believed also that PARP1—DNA trapping is an effective mechanism for selectively killing tumor cells having HRD. An unmet medical need therefore exists for effective and safe PARP inhibitors, especially PARP inhibitors having selectivity for PARP1, and a stable form of a PARP1 inhibitor.

### Summary

Provided herein are salts and solid forms of a compound that inhibits PARP1.

Provided herein are salts and solid forms, such as crystalline forms, of Compound I:

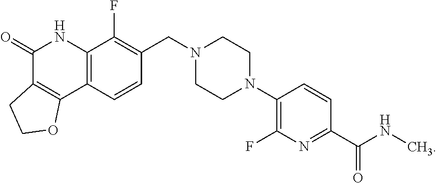 [SMILES: CNC(=O)c1ccc(N2CCN(Cc3ccc4c5c(c(=O)[nH]c4c3F)CCO5)CC2)c(F)n1]

Also disclosed herein is a pharmaceutical composition comprising a crystalline form disclosed herein, and a pharmaceutically acceptable excipient.

Also disclosed herein is a method of treating cancer in a subject in need thereof, the method comprising administering a crystalline form disclosed herein.

In some embodiments, the cancer is breast cancer, ovarian cancer, pancreatic cancer, prostate cancer, a hematological cancer, gastrointestinal cancer, or lung cancer.

Also disclosed herein is a method of treating a cancer comprising a BRCA1 and/or a BRCA2 mutation in a subject in need thereof, the method comprising administering a crystalline form disclosed herein.

In some embodiments, the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

### BRIEF DESCRIPTION OF THE FIGURES

FIG. 1A shows an X-ray powder diffraction (XRPD) pattern for Compound I Form I.

FIG. 1B shows a differential scanning calorimetry (DSC) curve and a thermogravimetric analysis (TGA) for Compound I Form I.

FIG. 1C shows a dynamic vapor sorption (DVS) curve for Compound I Form I.

FIG. 2A shows an XRPD pattern for Compound I Form II.

FIG. 2B shows a DSC and TGA for Compound I Form II.

FIG. 3A shows an XRPD pattern for Compound I Form III.

FIG. 3B shows a DSC and TGA for Compound I Form III.

FIG. 3C shows a DVS curve for Compound I Form III.

FIG. 4A shows an XRPD pattern for Compound I Form IV.

FIG. 4B shows a DSC and TGA for Compound I Form IV.

FIG. 5A shows an XRPD pattern for Compound I Form V.

FIG. 5B shows a DSC and TGA for Compound I Form V.

FIG. 6A shows an XRPD pattern for Compound I Form VI.

FIG. 6B shows a DSC and TGA for Compound I Form VI.

FIG. 7A shows an XRPD pattern for Compound I Form VII.

FIG. 7B shows a DSC and TGA for Compound I Form VII.

FIG. 8A shows an XRPD pattern for Compound I Form VIII.

FIG. 8B shows a DSC and TGA for Compound I Form VIII.

FIG. 9A shows an XRPD pattern for Compound I Form IX.

FIG. 9B shows a DSC and TGA for Compound I Form IX.

FIG. 10A shows an XRPD pattern for Compound I Form X.

FIG. 11A shows an XRPD pattern for Compound I Form XI.

FIG. 11B shows a DSC and TGA for Compound I Form XI.

FIG. 12A shows an XRPD pattern for Compound I Form XII.

FIG. 12B shows a DSC and TGA for Compound I Form XII.

FIG. 13A shows an XRPD pattern for Compound I Form XIII.

FIG. 13B shows a DSC and TGA for Compound I Form XIII.

FIG. 14A shows an XRPD pattern for Compound I Form XIV.

FIG. 15A shows an XRPD pattern for Compound I Phosphate Form I.

FIG. 15B shows a DSC thermogram for Compound I Phosphate Form I.

FIG. 15C shows a TGA for Compound I Phosphate Form I.

FIG. 15D shows an XRPD pattern for Compound I Phosphate Form I.

FIG. 15E shows a DSC and TGA for Compound I Phosphate Form I.

FIG. 15F shows a DSC and TGA for Compound I Phosphate Form I.

FIG. 15G shows a DVS curve for Compound I Phosphate Form I.

FIG. 16A shows an XRPD pattern for Compound I Fumarate Form I.

FIG. 16B shows a DSC thermogram for Compound I Fumarate Form I.

FIG. 16C shows a TGA for Compound I Fumarate Form I.

FIG. 16D shows an XRPD pattern for Compound I Fumarate Form I.

FIG. 16E shows a DSC and TGA for Compound I Fumarate Form I.

FIG. 16F shows a DVS curve for Compound I Fumarate Form I.

FIG. 16G shows a DSC thermogram for Compound I Fumarate Form I.

FIG. 17A shows an XRPD pattern for Compound I HCl Form I.

FIG. 17B shows a DSC and TGA for Compound I HCl Form I.

FIG. 18A shows an XRPD pattern for Compound I HCl Form II.

FIG. 18B shows a DSC and TGA for Compound I HCl Form II.

FIG. 19A shows an XRPD pattern for Compound I Sulfate Form I.

FIG. 19B shows a DSC and TGA for Compound I Sulfate Form I.

FIG. 20A shows an XRPD pattern for Compound I Maleate Form I.

FIG. 20B shows a DSC and TGA for Compound I Maleate Form I.

FIG. 21A shows an XRPD pattern for Compound I Phosphate Form II.

FIG. 21B shows a DSC and TGA for Compound I Phosphate Form II.

FIG. 22A shows an XRPD pattern for Compound I Succinate Form I.

FIG. 22B shows a DSC and TGA for Compound I Succinate Form I.

FIG. 23A shows an XRPD pattern for Compound I Tosylate Form I.

FIG. 23B shows a DSC and TGA for Compound I Tosylate Form I.

FIG. 24A shows an XRPD pattern for Compound I Mesylate Form I.

FIG. 24B shows a DSC and TGA for Compound I Mesylate Form I.

FIG. 25A shows an XRPD pattern for Compound I Phosphate Form III.

FIG. 26A shows an XRPD pattern for Compound I Phosphate Form IV.

FIG. 27A shows an XRPD pattern for Compound I Phosphate Form V.

FIG. 27B shows a DSC and TGA for Compound I Phosphate Form V.

FIG. 28A shows an XRPD pattern for Compound I Phosphate Form VI.

FIG. 28B shows a DSC and TGA for Compound I Phosphate Form VI.

FIG. 29A shows an XRPD pattern for Compound I Phosphate Form VII.

FIG. 29B shows a DSC and TGA for Compound I Phosphate Form VII.

FIG. 30A shows an XRPD pattern for Compound I Malonate Acetonitrile Solvate Form I.

FIG. 31A shows an XRPD pattern for Compound I Malonate Form I.

FIG. 31B shows a DSC thermogram for Compound I Malonate Form I.

FIG. 31C shows a TGA for Compound I Malonate Form I.

FIG. 32A shows an XRPD pattern for Compound I Citrate Methanol Solvate Form I.

FIG. 33A shows an XRPD pattern for Compound I Citrate Form I.

FIG. 34A shows an XRPD pattern for Compound I Citrate Form II.

FIG. 34B shows a DSC thermogram for Compound I Citrate Form II.

FIG. 34C shows a TGA for Compound I Citrate Form II.

FIG. 35A shows an XRPD pattern for Compound I Oxalate Acetonitrile Solvate Form I.

FIG. 36A shows an XRPD pattern for Compound I Oxalate Form I.

FIG. 37A shows an XRPD pattern for Compound I Fumarate Form II.

FIG. 37B shows a DSC thermogram for Compound I Fumarate Form II.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

Forms of Compound I or salts, co-crystals, solvates, or hydrates thereof are provided herein. In some embodiments, reference to a form of Compound I or a salt, co-crystal, solvate, or hydrate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt, co-crystal, solvate, or hydrate thereof is present in a composition in the designated form. For instance, in some embodiments, reference to of Compound I Form I means that at least 50%, at least 55%, at least 60%, at least 65%, at least 70%, at least 75%, at least 80%, at least 85%, at least 90%, at least 95%, or at least 99% of Compound I is present in a composition as Form I.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

As used herein, the term “salt” refers to a compound formed by the reaction of an acid and a base, resulting in the formation of a positively charged cation and a negatively charged anion. In general, a salt is defined as a compound that is formed by the combination of positively and negatively charged ions, where the charges of the ions result in a neutral compound. Salts can be either inorganic or organic. As used herein, the term “salt” includes partially or fully ionized salt forms. In some embodiments, the salt is fully ionized.

The term “co-crystal” refers to a molecular complex of a compound disclosed herein and one or more non-ionized co-crystal formers connected via non-covalent interactions. In some embodiments, the co-crystals disclosed herein may include a non-ionized form of Compound I (e.g., Compound I free form) and one or more non-ionized co-crystal formers, where non-ionized Compound I and the co-crystal former(s) are connected through non-covalent interactions. In some embodiments, co-crystals disclosed herein may include an ionized form of Compound I (e.g., a salt of Compound I) and one or more non-ionized co-crystals formers, where ionized Compound I and the co-crystal former(s) are connected through non-covalent interactions. Co-crystals may additionally be present in anhydrous, solvated or hydrated forms. In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, increased stability, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “co-crystal former” or “co-former” refers to one or more pharmaceutically acceptable bases or pharmaceutically acceptable acids disclosed herein in association with Compound I, or any other compound disclosed herein. Such bases include ethanolamine, diethanolamine, triethanolamine, tromethamine, N-methylglucamine, and the like. Such acids include inorganic acids such as hydrochloric acid, sulfuric acid, nitric acid, phosphoric acid, and the like; or organic acids such as formic acid, acetic acid, propionic acid, glycolic acid, pyruvic acid, lactic acid, malonic acid, maleic acid, tartaric acid, citric acid, methanesulfonic acid, ethanesulfonic acid, gluconic acid, glutamic acid, salicylic acid, stearic acid, and the like.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, acetonitrile, methanol, N,N-dimethylformamide, tetrahydrofuran, 2-methyltetrahydrofuran, dimethylsulfoxide, and water. In general, the solvated forms are equivalent to unsolvated forms and are encompassed within the scope of the present disclosure.

The term “desolvated” refers to a form that is a solvate as described herein, and from which solvent molecules have been partially or completely removed. Desolvation techniques to produce desolvated forms include, without limitation, exposure of a form (solvate) to a vacuum, subjecting the solvate to elevated temperature, exposing the solvate to a stream of gas, such as air or nitrogen, or any combination thereof. Thus, a desolvated or “unsolvated” form can be “anhydrous”, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

“Physically stable” as used herein indicates that a particular form of a compound does not change into one or more different physical forms (e.g., a different solid form as measured by XRPD, DSC, TGA, etc) when subjected to specified conditions (e.g., at room temperature and/or ambient humidity) for a specified period of time (e.g., 1 day, 2 days, 3 days, 1 week, 2 weeks, 3 weeks, 1 month, 2 months, 6 months, 12 months, or longer). In some embodiments, less than 25% of a particular form of a compound changes into one or more different physical forms when subjected to specified conditions. In some embodiments, less than about 20%, less than about 15%, less than about 10%, less than about 5%, less than about 1%, or less than about 0.5% of the particular form of a compound changes into one or more different physical forms when subjected to specified conditions. In some embodiments, no detectable amount of the particular form of a compound changes into one or more different physical forms when subjected to specified conditions.

“Chemically stable” as used herein indicates that the chemical structure of a particular compound does not change into another compound (e.g., decompose) when subjected to specified conditions (e.g., at room temperature and/or ambient humidity) for a specified period of time (e.g., 1 day, 2 days, 3 days, 1 week, 2 weeks, 3 weeks, 1 month, 2 months, 6 months, 12 months, or longer). In some embodiments, less than 25% of a particular form of a compound changes into one or more different compounds when subjected to specified conditions. In some embodiments, less than about 20%, less than about 15%, less than about 10%, less than about 5%, less than about 1%, or less than about 0.5% of the particular form of a compound changes into one or more different compounds when subjected to specified conditions. In some embodiments, no detectable amount of the particular form of a compound changes into one or more different compounds when subjected to specified conditions.

“Substantially pure (form of a polymorph),” in some embodiments, means that in the referenced material, at least 99.9% of the material is the referenced polymorph. “Substantially pure (form of a polymorph),” in some embodiments, means that in the referenced material, at least 99.5% of the material is the referenced polymorph. “Substantially pure (form of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure (form of a polymorph),” in some embodiments, means that in the referenced material, at least 97% of the material is the referenced polymorph. “Substantially pure (form of a polymorph),” in some embodiments, means that in the referenced material, at least 96% of the material is the referenced polymorph. “Substantially pure (form of a polymorph),” in some embodiments, means that in the referenced material, at least 95% of the material is the referenced polymorph.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to a salt that retains the biological effectiveness and properties of the given compound, and which are not biologically or otherwise undesirable. The term “pharmaceutically acceptable solvate” of a given compound likewise refers to a solvate of a given compound or salt thereof that retains the biological effectiveness and properties of the given compound or salt thereof, and which are not biologically or otherwise undesirable.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

As used herein, a “disease or disorder associated with PARP” or, alternatively, “a PARP-mediated disease or disorder” means any disease or other deleterious condition in which PARP, or a mutant thereof, is known or suspected to play a role.

As used herein, a “disease or disorder associated with PARP1” or, alternatively, “a PARP1-mediated disease or disorder” means any disease or other deleterious condition in which PARP1, or a mutant thereof, is known or suspected to play a role.

In some embodiments, the phrase “substantially shown in Figure” or “substantially as shown in Figure” as applied to an X-ray powder diffractogram is meant to include a variation of ±0.2 °2θ or ±0.1 °2θ, as applied to DSC thermograms is meant to include a variation of ±3° Celsius, and as applied to thermogravimetric analysis (TGA) is meant to include a variation of ±2% in weight loss. In some embodiments, the phrase “substantially shown in Figure” or “substantially as shown in Figure” as applied to DVS curves is meant to include a variation of ±5%.

### Compound I

The compound, 6-fluoro-5-[4-({6-fluoro-4-oxo-2H,3H,5H-furo[3,2-c]quinolin-7-yl} methyl) piperazin-1-yl]-N-methylpyridine-2-carboxamide, designated herein as Compound I, has the following formula:

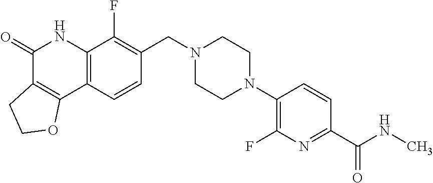 [SMILES: CNC(=O)c1ccc(N2CCN(Cc3ccc4c5c(c(=O)[nH]c4c3F)CCO5)CC2)c(F)n1]

Compound I is an inhibitor of PARP1. The synthesis and method of use thereof is described in PCT International Application Publication No. WO 2023/212219, which is hereby incorporated by reference in its entirety.

Unless otherwise specified, reference to Compound I is intended to encompass the compound per se, or a salt, such as a pharmaceutically acceptable salt, stereoisomer, mixture of stereoisomers, solid form, co-crystal, solvate, and/or hydrate thereof.

### Salts and Forms of Compound I

The present disclosure provides salts and solid forms, such as crystalline forms, of the compound, 6-fluoro-5-[4-({6-fluoro-4-oxo-2H,3H,5H-furo[3,2-c]quinolin-7-yl} methyl) piperazin-1-yl]-N-methylpyridine-2-carboxamide (hereinafter “Compound I”), and salts, co-crystals, solvates, or hydrates thereof. Crystalline forms of Compound I and salts, co-crystals, solvates, or hydrates thereof, and other forms (e.g., amorphous forms) of Compound I and salts, co-crystals, solvates, or hydrates thereof are collectively referred to herein as “forms of Compound I.”

In some embodiments, Compound I is in free form, e.g., a free base. In some embodiments, Compound I is a salt. In some embodiments, Compound I is a pharmaceutically acceptable salt. In some embodiments, Compound I is a solvate. In some embodiments, Compound I is a hydrate. In some embodiments, Compound I is unsolvated. In some embodiments, Compound I is an anhydrate. In some embodiments, provided is a substantially pure form of a solid form of Compound I as described herein. In some embodiments, provided is a substantially pure form of a crystalline form of Compound I as described herein. In some embodiments, provided is an amorphous form of Compound I.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.: 3635-3645 (with respect to crystal engineering, if instructions are not very precise and/or if other external factors affect the process, the result can be unpredictable); Jones et al., 2006, “Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

The polymorphs made according to the methods of the disclosure may be characterized by any methodology according to the art. For example, the polymorphs made according to the methods of the disclosure may be characterized by X-ray powder diffraction (XRPD), differential scanning calorimetry (DSC), thermogravimetric analysis (TGA), hot-stage microscopy, and/or spectroscopy (e.g., Raman, solid-state nuclear magnetic resonance (ssNMR), and infrared (IR)). In some embodiments, crystallinity of a solid form is determined by X-Ray Powder Diffraction (XRPD).

XRPD: Polymorphs according to the disclosure may be characterized by XRPD. The relative intensities of XRPD peaks can vary, depending upon the particle size, the sample preparation technique, the sample mounting procedure and the particular instrument employed. Moreover, instrument variation and other factors can affect the 20 values. Therefore, the XRPD peak assignments can vary, for example by plus or minus 0.2 degrees or by plus or minus 0.1 degrees.

DSC: Polymorphs according to the disclosure can also be identified by its characteristic DSC thermograms. For DSC, it is known that the temperatures observed will depend upon the rate of temperature change as well as sample preparation technique and the particular instrument employed. Thus, the values reported herein relating to DSC thermograms can vary, for example by plus or minus 4° C.

TGA: The polymorphic forms of the disclosure may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of Compound I are useful in the production of medicinal preparations and can be obtained by means of a crystallization process to produce crystalline and semi-crystalline forms or a solidification process to obtain the amorphous form. In some embodiments, the crystallization is carried out by either generating the desired compound (for example, Compound I) in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. In some embodiments, the crystallization comprises addition of a seed form of a desired polymorph. The crystallization or solidification may be followed by drying carried out under controlled conditions until the desired water content is reached in the end polymorphic form. Accordingly, some embodiments provide for methods of preparing a solid form or crystalline form as described herein.

### Forms of Compound I

### Compound I Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form I (Compound I Form I) characterized by an X-ray powder diffractogram comprising peaks (±0.2°) at 8.1, 9.4, and 19.7 °2θ as determined on a diffractometer using Cu-Kα radiation.

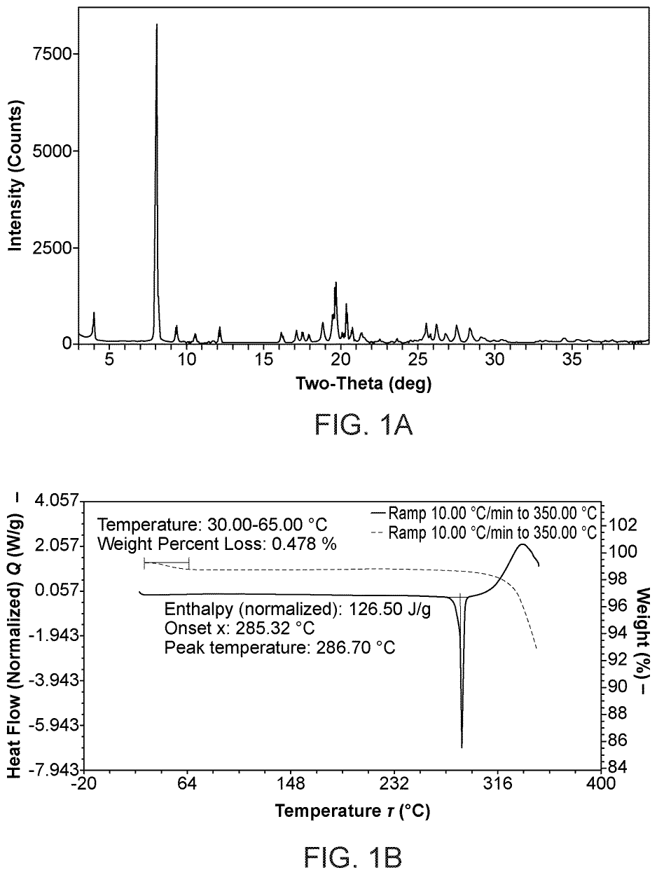

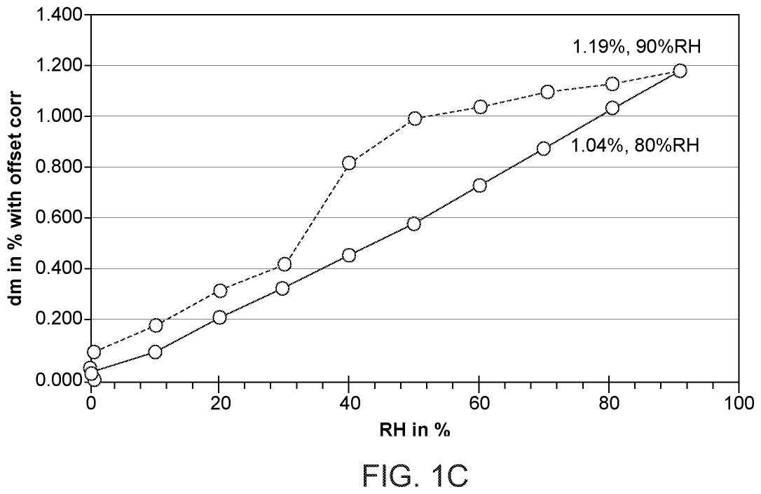

In some embodiments, Compound I Form I is further characterized by:
i) one or more peaks at (±0.2°) at 4.0, 12.2, or 18.8 °2θ;
ii) a diffractogram substantially as shown in FIG. 1A;
iii) a differential scanning calorimetry (DSC) curve comprising an endotherm at about 285° C. (onset temperature);
iv) a differential scanning calorimetry (DSC) curve substantially as shown in FIG. 1B;
v) thermogravimetric analysis (TGA) showing a weight loss of about 0.5 wt % up to about 65° C.;
vi) thermogravimetric analysis (TGA) comprising a thermogram substantially as shown in FIG. 1B; or
vii) a dynamic vapor sorption (DVS) curve showing about 1.04% water uptake at 80% relative humidity (RH) or about 1.19% water uptake at 90% RH; or
viii) a DVS curve substantially as shown in FIG. 1C.

In some embodiments, Compound I Form I is further characterized by an X-ray powder diffractogram comprising one or more peaks at (±0.2°) at 4, 12.2, or 18.8 °2θ as determined on a diffractometer using Cu-Kα radiation. In some embodiments, Compound I Form I is further characterized by an X-ray powder diffractogram comprising one or more peaks at (±0.2°) at 10.6, 16.1, or 20.4 °2θ as determined on a diffractometer using Cu-Kα radiation. In some embodiments, Compound I Form I is further characterized by a diffractogram substantially as shown in FIG. 1A.

In some embodiments, Compound I Form I is further characterized by a DSC curve comprising an endotherm at about 285° C. (onset temperature). In some embodiments, Compound I Form I is further characterized by a DSC curve comprising an endotherm at about 287° C. (peak). In some embodiments, Compound I Form I is further characterized by a DSC curve substantially as shown in FIG. 1B.

In some embodiments, Compound I Form I is further characterized by TGA showing a weight loss of about 0.5 wt % up to about 65° C. In some embodiments, Compound I Form I is further characterized by TGA comprising a thermogram substantially as shown in FIG. 1B.

In some embodiments, Compound I Form I is further characterized by a DVS curve showing about 1.04% water uptake at 80% relative humidity (RH) or about 1.19% water uptake at 90% RH. In some embodiments, Compound I Form I is further characterized by a DVS curve substantially as shown in FIG. 1C.

In some embodiments, Compound I Form I is an anhydrate.

### Compound I Form II

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form II (Compound I Form II) characterized by an X-ray powder diffractogram comprising peaks (±0.2°) at 6.4, 9.1, and 18.7 °2θ as determined on a diffractometer using Cu-Kα radiation.

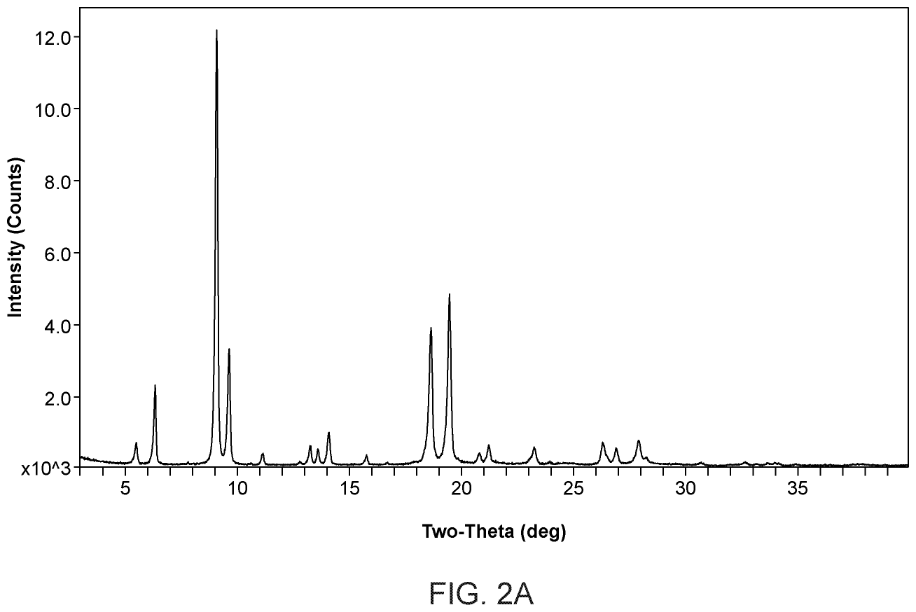

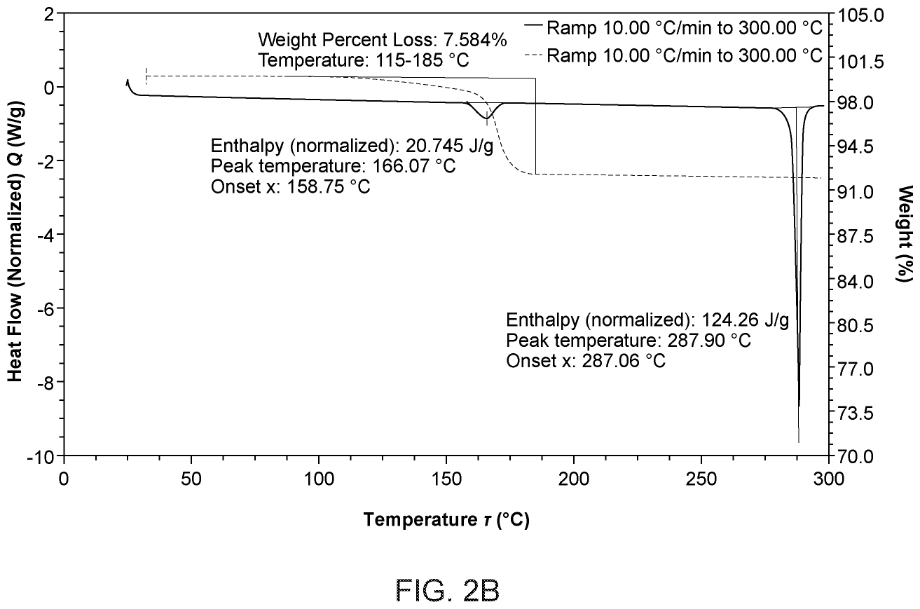

In some embodiments, Compound I Form II is further characterized by:
i) one or more peaks at (±0.2°) at 9.7, 14.1, or 19.5 °2θ;
ii) a diffractogram substantially as shown in FIG. 2A;
iii) a differential scanning calorimetry (DSC) curve comprising an endotherm at about 159° C. (onset temperature) and an endotherm at about 287° C. (onset temperature);
iv) a differential scanning calorimetry (DSC) curve substantially as shown in FIG. 2B;
v) thermogravimetric analysis (TGA) showing a weight loss of about 7.6 wt % up to about 185° C.; or
vi) thermogravimetric analysis (TGA) comprising a thermogram substantially as shown in FIG. 2B.

In some embodiments, Compound I Form II is further characterized by an X-ray powder diffractogram comprising one or more peaks at (±0.2°) at 9.7, 14.1, or 19.5 °2θ. In some embodiments, Compound I Form II is further characterized by an X-ray powder diffractogram comprising one or more peaks at (±0.2°) at 5.5, 11.2, or 23.3 °2θ as determined on a diffractometer using Cu-Kα radiation. In some embodiments, Compound I Form II is further characterized by a diffractogram substantially as shown in FIG. 2A.

In some embodiments, Compound I Form II is further characterized by a DSC curve comprising an endotherm at about 159° C. (onset temperature) and an endotherm at about 287° C. (onset temperature). In some embodiments, Compound I Form II is further characterized by a DSC curve comprising an endotherm at about 166° C. (peak) and an endotherm at about 288° C. (peak). In some embodiments, Compound I Form II is further characterized by a DSC curve substantially as shown in FIG. 2B.

In some embodiments, Compound I Form II is further characterized by a TGA showing a weight loss of about 7.6 wt % up to about 185° C. In some embodiments, Compound I Form II is further characterized by a TGA comprising a thermogram substantially as shown in FIG. 2B.

In some embodiments, Compound I Form II is a solvate. In some embodiments, Compound I Form II is a toluene solvate.

### Compound I Form III

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form III (Compound I Form III) characterized by an X-ray powder diffractogram comprising peaks (±0.2°) at 4.7, 9.5, and 12.6 °2θ as determined on a diffractometer using Cu-Kα radiation.

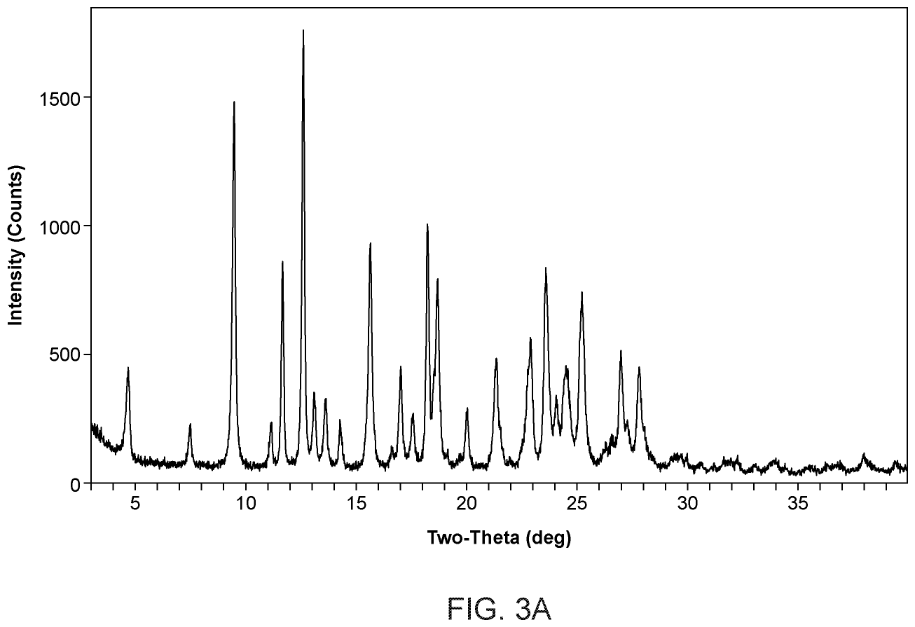

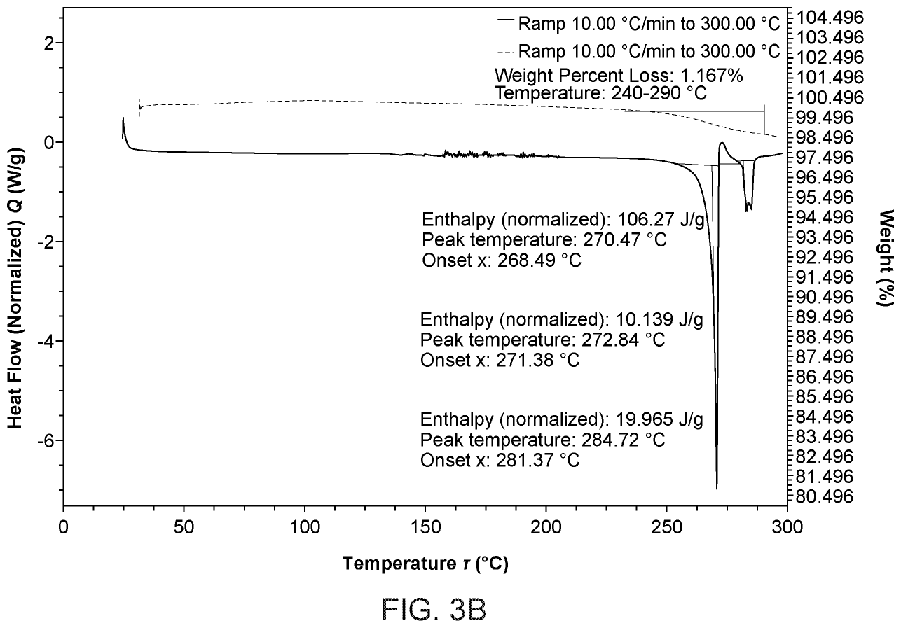

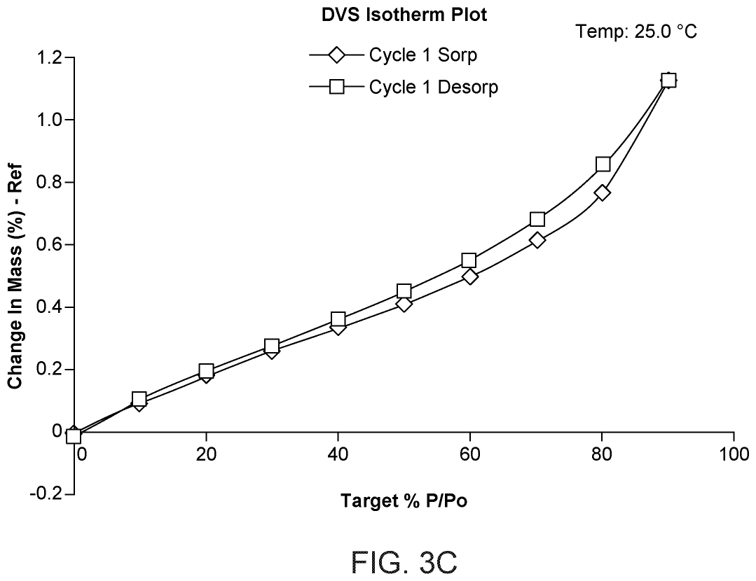

In some embodiments, Compound I Form III is further characterized by:
i) one or more peaks at (±0.2°) at 7.5, 11.7, or 18.3 °2θ;
ii) a diffractogram substantially as shown in FIG. 3A;
iii) a differential scanning calorimetry (DSC) curve comprising an endotherm at about 268° C. (onset temperature) and an endotherm at about 281° C. (onset temperature);
iv) a differential scanning calorimetry (DSC) curve substantially as shown in FIG. 3B;
v) thermogravimetric analysis (TGA) showing a weight loss of about 1.2 wt % up to about 290° C.;
vi) thermogravimetric analysis (TGA) comprising a thermogram substantially as shown in FIG. 3B;
vii) a dynamic vapor sorption (DVS) curve showing about 0.77% water uptake at 80% RH or about 1.13% at 90% RH; or
viii) a DVS curve substantially as shown in FIG. 3C.

In some embodiments, Compound I Form III is further characterized by an X-ray powder diffractogram comprising one or more peaks at (±0.2°) at 7.5, 11.7, or 18.3 °2θ. In some embodiments, Compound I Form III is further characterized by an X-ray powder diffractogram comprising one or more peaks at (±0.2°) at 11.2, 15.7, or 23.6 °2θ as determined on a diffractometer using Cu-Kα radiation. In some embodiments, Compound I Form III is further characterized by a diffractogram substantially as shown in FIG. 3A.

In some embodiments, Compound I Form III is further characterized by a DSC curve comprising an endotherm at about 268° C. (onset temperature) and an endotherm at about 281° C. (onset temperature). In some embodiments, Compound I Form III is further characterized by a DSC curve comprising an endotherm at about 268° C. (onset temperature), an exotherm at about 271° C. (onset temperature), and an endotherm at about 281° C. (onset temperature). In some embodiments, Compound I Form III is further characterized by a DSC curve comprising an endotherm at about 270° C. (peak), an exotherm at about 273° C. (peak), and an endotherm at about 285° C. (peak). In some embodiments, Compound I Form III is further characterized by a DSC curve substantially as shown in FIG. 3B.

In some embodiments, Compound I Form III is further characterized by TGA showing a weight loss of about 1.2 wt % up to about 290° C. In some embodiments, Compound I Form III is further characterized by TGA comprising a thermogram substantially as shown in FIG. 3B.

In some embodiments, Compound I Form III is further characterized by a DVS curve showing about 0.77% water uptake at 80% RH or about 1.13% at 90% RH. In some embodiments, Compound I Form III is further characterized by a DVS curve substantially as shown in FIG. 3C.

In some embodiments, Compound I Form III is an anhydrate.

### Compound I Form IV

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form IV (Compound I Form IV) characterized by an X-ray powder diffractogram comprising peaks (±0.2°) at 4.9, 9.9, and 16.6 °2θ as determined on a diffractometer using Cu-Kα radiation.

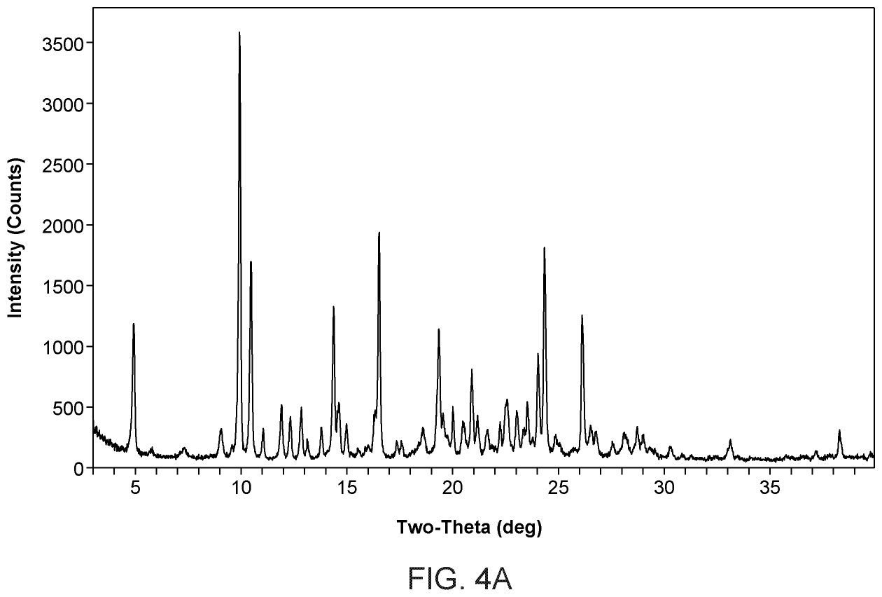

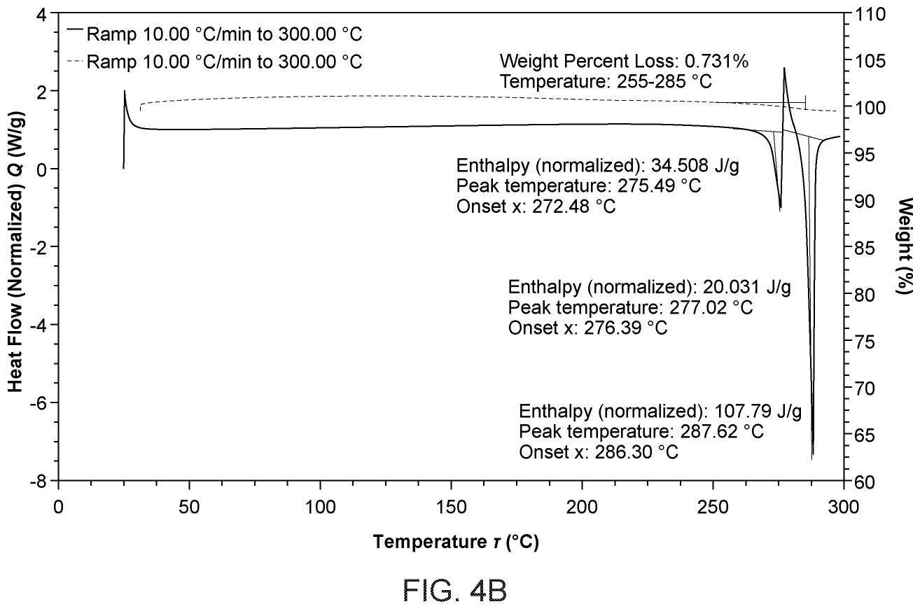

In some embodiments, Compound I Form IV is further characterized by:
i) one or more peaks at (±0.2°) at 10.5, 14.4, or 19.4 °2θ;
ii) a diffractogram substantially as shown in FIG. 4A;
iii) a differential scanning calorimetry (DSC) curve comprising an endotherm at about 272° C. (onset temperature) and an endotherm at about 286° C. (onset temperature);
iv) a differential scanning calorimetry (DSC) curve substantially as shown in FIG. 4B;
v) thermogravimetric analysis (TGA) showing a weight loss of about 0.7 wt % up to about 285° C.; or
vi) thermogravimetric analysis (TGA) comprising a thermogram substantially as shown in FIG. 4B.

In some embodiments, Compound I Form IV is further characterized by an X-ray powder diffractogram comprising one or more peaks at (±0.2°) at 10.5, 14.4, or 19.4 °2θ. In some embodiments, Compound I Form IV is further characterized by an X-ray powder diffractogram comprising one or more peaks at (±0.2°) at 9.1, 11.9, or 20.9 °2θ as determined on a diffractometer using Cu-Kα radiation. In some embodiments, Compound I Form IV is further characterized by a diffractogram substantially as shown in FIG. 4A.

In some embodiments, Compound I Form IV is further characterized by DSC curve comprising an endotherm at about 272° C. (onset temperature) and an endotherm at about 286° C. (onset temperature). In some embodiments, Compound I Form IV is further characterized by DSC curve comprising an endotherm at about 272° C. (onset temperature), an exotherm at 276° C. (onset temperature), and an endotherm at about 286° C. (onset temperature). In some embodiments, Compound I Form IV is further characterized by DSC curve comprising an endotherm at about 275° C. (peak), an exotherm at 277° C. (peak), and an endotherm at about 288° C. (peak). In some embodiments, Compound I Form IV is further characterized by a DSC curve substantially as shown in FIG. 4B.

In some embodiments, Compound I Form IV is further characterized by TGA showing a weight loss of about 0.7 wt % up to about 285° C. In some embodiments, Compound I Form IV is further characterized by TGA comprising a thermogram substantially as shown in FIG. 4B.

In some embodiments, Compound I Form IV is an anhydrate.

### Compound I Form V

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form V (Compound I Form V) characterized by an X-ray powder diffractogram comprising peaks (±0.2°) at 5.7, 9.3, and 14.4 °2θ as determined on a diffractometer using Cu-Kα radiation.

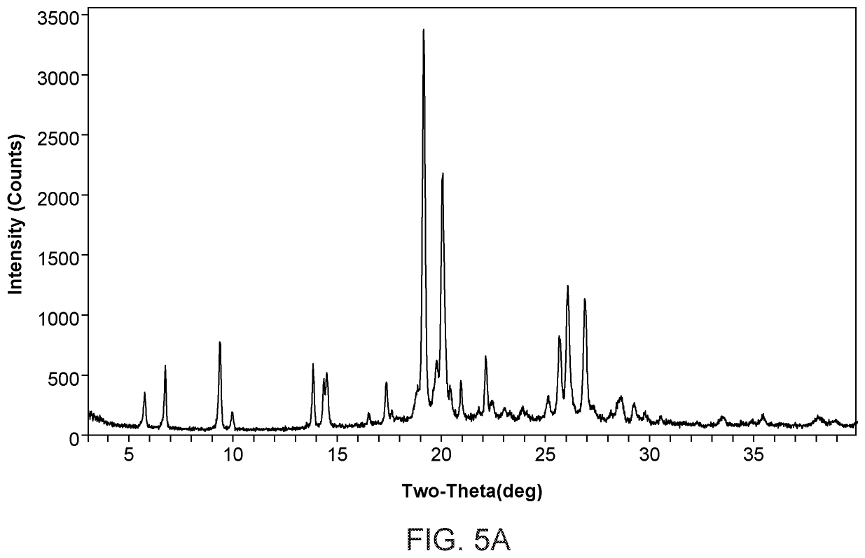

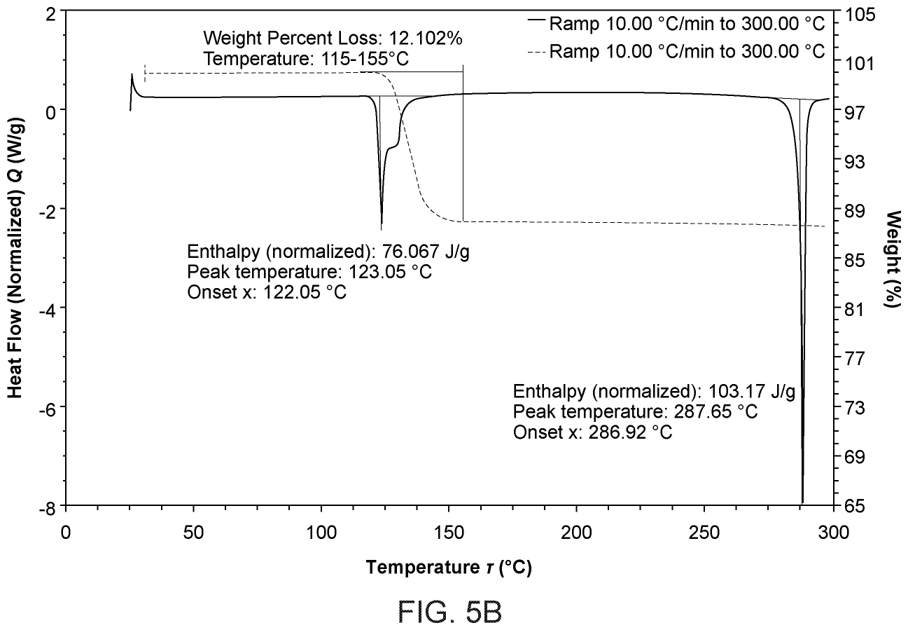

In some embodiments, Compound I Form V is further characterized by:
i) one or more peaks at (±0.2°) at 6.7, 13.8, or 17.4 °2θ;
ii) a diffractogram substantially as shown in FIG. 5A;
iii) a differential scanning calorimetry (DSC) curve comprising an endotherm at about 122° C. (onset temperature) and an endotherm at about 287° C. (onset temperature);
iv) a differential scanning calorimetry (DSC) curve substantially as shown in FIG. 5B;
v) thermogravimetric analysis (TGA) showing a weight loss of about 12.1 wt % up to about 155° C.; or
vi) thermogravimetric analysis (TGA) comprising a thermogram substantially as shown in FIG. 5B.

In some embodiments, Compound I Form V is further characterized by an X-ray powder diffractogram comprising one or more peaks at (±0.2°) at 6.7, 13.8, or 17.4 °2θ. In some embodiments, Compound I Form V is further characterized by an X-ray powder diffractogram comprising one or more peaks at (±0.2°) at 9.9, 19.1, or 20.0 °2θ as determined on a diffractometer using Cu-Kα radiation. In some embodiments, Compound I Form V is further characterized by a diffractogram substantially as shown in FIG. 5A.

In some embodiments, Compound I Form V is further characterized by a DSC curve comprising an endotherm at about 122° C. (onset temperature) and an endotherm at about 287° C. (onset temperature). In some embodiments, Compound I Form V is further characterized by a DSC curve comprising an endotherm at about 123° C. (peak) and an endotherm at about 288° C. (peak). In some embodiments, Compound I Form V is further characterized by a DSC curve substantially as shown in FIG. 5B.

In some embodiments, Compound I Form V is further characterized by TGA showing a weight loss of about 12.1 wt % up to about 155° C. In some embodiments, Compound I Form V is further characterized by TGA comprising a thermogram substantially as shown in FIG. 5B.

In some embodiments, Compound I Form V is a solvate. In some embodiments, Compound I Form V is a dimethylsulfoxide (DMSO) solvate.

### Compound I Form VI

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form VI (Compound I Form VI) characterized an X-ray powder diffractogram comprising peaks (±0.2°) at 7.7, 11.0, and 15.4 °2θ as determined on a diffractometer using Cu-Kα radiation.

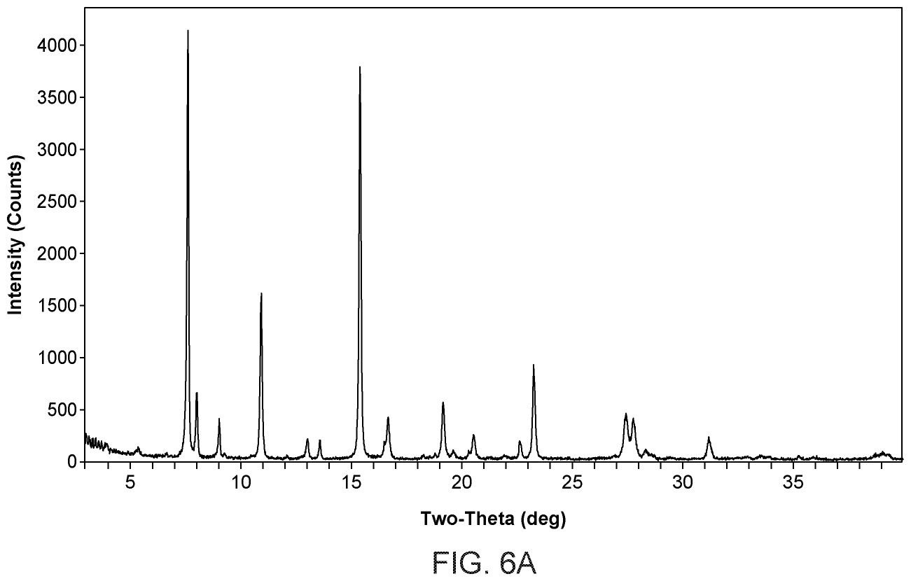

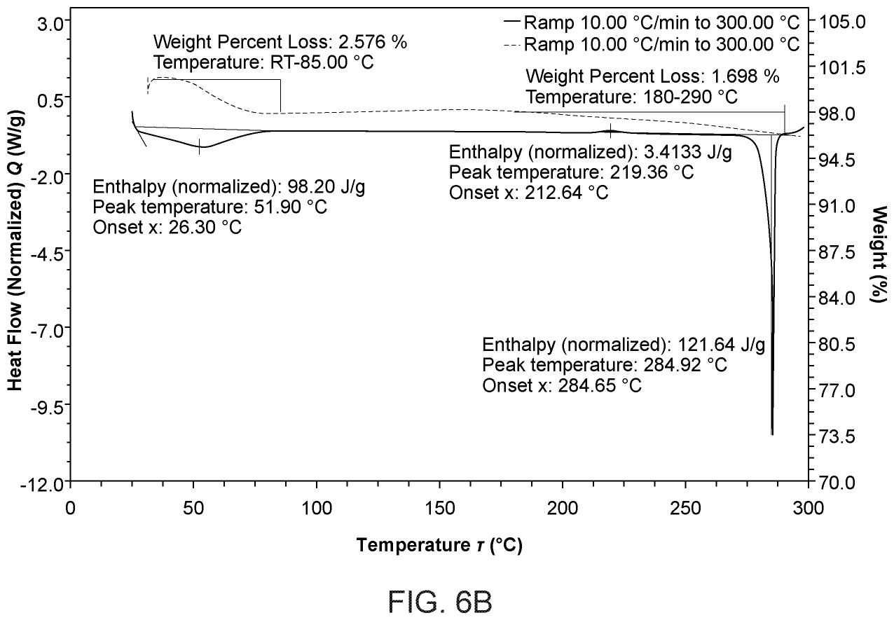

In some embodiments, Compound I Form VI is further characterized by:
i) one or more peaks at (±0.2°) at 8.1, 16.7, or 19.2 °2θ;
ii) a diffractogram substantially as shown in FIG. 6A;
iii) a differential scanning calorimetry (DSC) curve comprising an endotherm at about 26° C. (onset temperature) and an endotherm at about 285° C. (onset temperature);
iv) a differential scanning calorimetry (DSC) curve substantially as shown in FIG. 6B;
v) thermogravimetric analysis (TGA) showing a weight loss of about 4.3 wt % up to about 290° C.; or
vi) thermogravimetric analysis (TGA) comprising a thermogram substantially as shown in FIG. 6B.

In some embodiments, Compound I Form VI is further characterized by an X-ray powder diffractogram comprising one or more peaks at (±0.2°) at 8.1, 16.7, or 19.2 °2θ. In some embodiments, Compound I Form VI is further characterized by an X-ray powder diffractogram comprising one or more peaks at (±0.2°) at 9.1, 13.6, or 23.3 °2θ as determined on a diffractometer using Cu-Kα radiation. In some embodiments, Compound I Form VI is further characterized by a diffractogram substantially as shown in FIG. 6A.

In some embodiments, Compound I Form VI is further characterized by a DSC curve comprising an endotherm at about 26° C. (onset temperature) and an endotherm at about 285° C. (onset temperature). In some embodiments, Compound I Form VI is further characterized by a DSC curve comprising an endotherm at about 52° C. (peak) and an endotherm at about 285° C. (peak). In some embodiments, Compound I Form VI is further characterized by a DSC curve substantially as shown in FIG. 6B.

In some embodiments, Compound I Form VI is further characterized by TGA showing a weight loss of about 4.3 wt % up to about 290° C. In some embodiments, Compound I Form VI is further characterized by TGA showing a weight loss of about 2.6 wt % up to about 85° C. and a weight loss of about 1.7 wt % between about 180 to 290° C. In some embodiments, Compound I Form VI is further characterized by TGA comprising a thermogram substantially as shown in FIG. 6B.

In some embodiments, Compound I Form VI is a hydrate.

### Compound I Form VII

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form VII (Compound I Form VII) characterized by an X-ray powder diffractogram comprising peaks (±0.2°) at 8.1, 16.4, and 21.5 °2θ as determined on a diffractometer using Cu-Kα radiation.

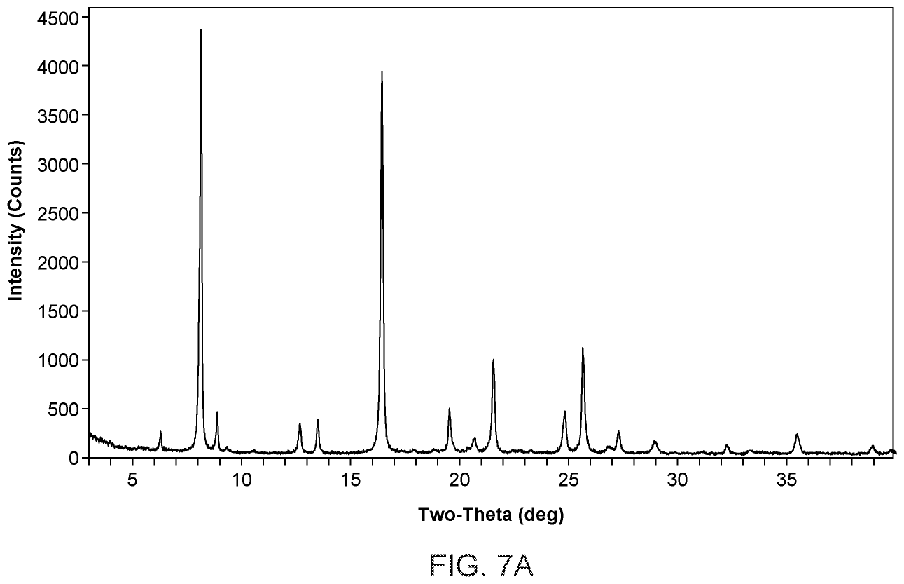

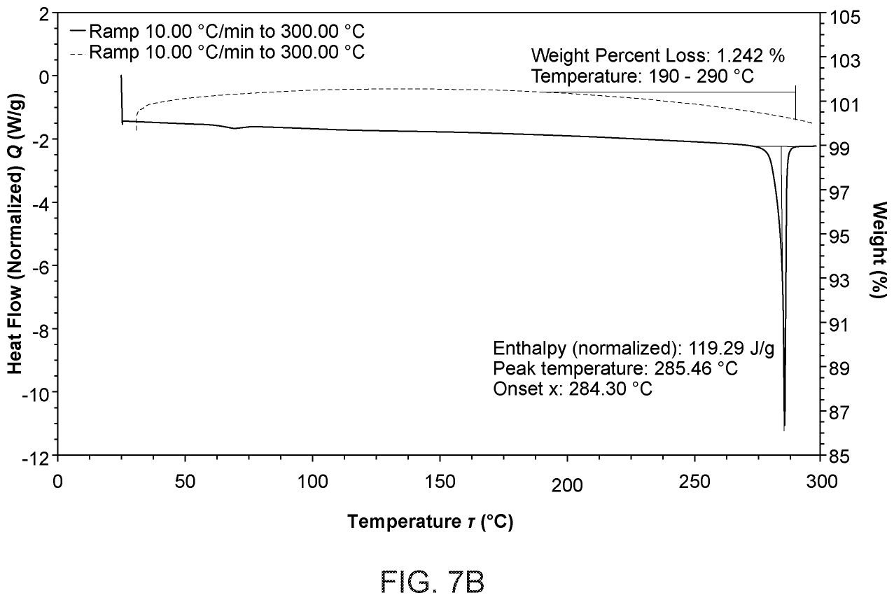

In some embodiments, Compound I Form VII is further characterized by:
i) one or more peaks at (±0.2°) at 8.9, 12.7, or 13.5 °2θ;
ii) a diffractogram substantially as shown in FIG. 7A;
iii) a differential scanning calorimetry (DSC) curve comprising an endotherm at about 284° C. (onset temperature);
iv) a differential scanning calorimetry (DSC) curve substantially as shown in FIG. 7B;
v) thermogravimetric analysis (TGA) showing a weight loss of about 1.2 wt % up to about 290° C.; or
vi) thermogravimetric analysis (TGA) comprising a thermogram substantially as shown in FIG. 7B.

In some embodiments, Compound I Form VII is further characterized by an X-ray powder diffractogram comprising one or more peaks at (±0.2°) at 8.9, 12.7, or 13.5 °2θ. In some embodiments, Compound I Form VII is further characterized by an X-ray powder diffractogram comprising one or more peaks at (±0.2°) at 6.3, 19.5, or 25.6 °2θ as determined on a diffractometer using Cu-Kα radiation. In some embodiments, Compound I Form VII is further characterized by a diffractogram substantially as shown in FIG. 7A.

In some embodiments, Compound I Form VII is further characterized by a DSC curve comprising an endotherm at about 284° C. (onset temperature). In some embodiments, Compound I Form VII is further characterized by a DSC curve comprising an endotherm at about 285° C. (peak). In some embodiments, Compound I Form VII is further characterized by a DSC curve substantially as shown in FIG. 7B.

In some embodiments, Compound I Form VII is further characterized by TGA showing a weight loss of about 1.2 wt % up to about 290° C. In some embodiments, Compound I Form VII is further characterized by TGA comprising a thermogram substantially as shown in FIG. 7B.

In some embodiments, Compound I Form VII is an anhydrate.

### Compound I Form VIII

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form VIII (Compound I Form VIII) characterized by an X-ray powder diffractogram comprising peaks (±0.2°) at 8.1, 10.1, and 18.0 °2θ as determined on a diffractometer using Cu-Kα radiation.

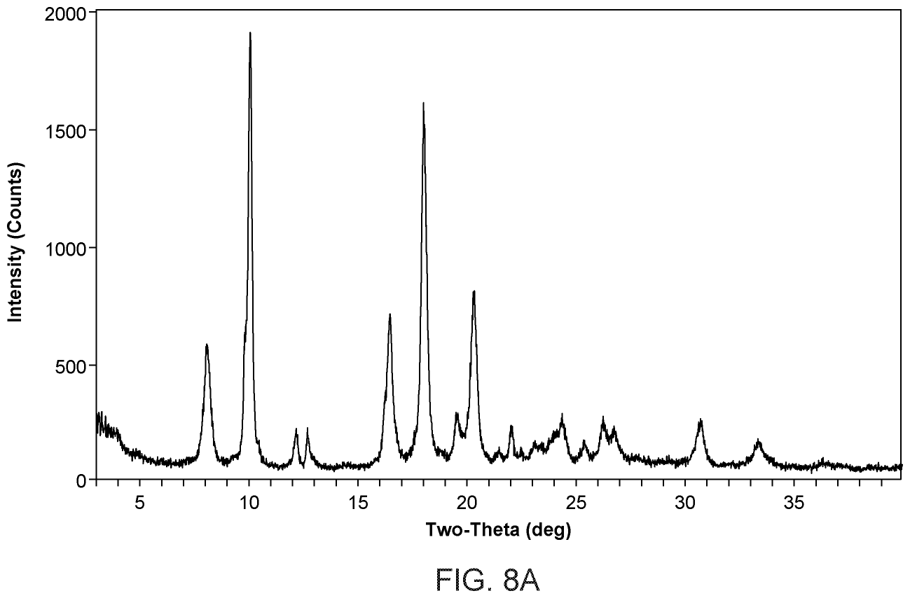

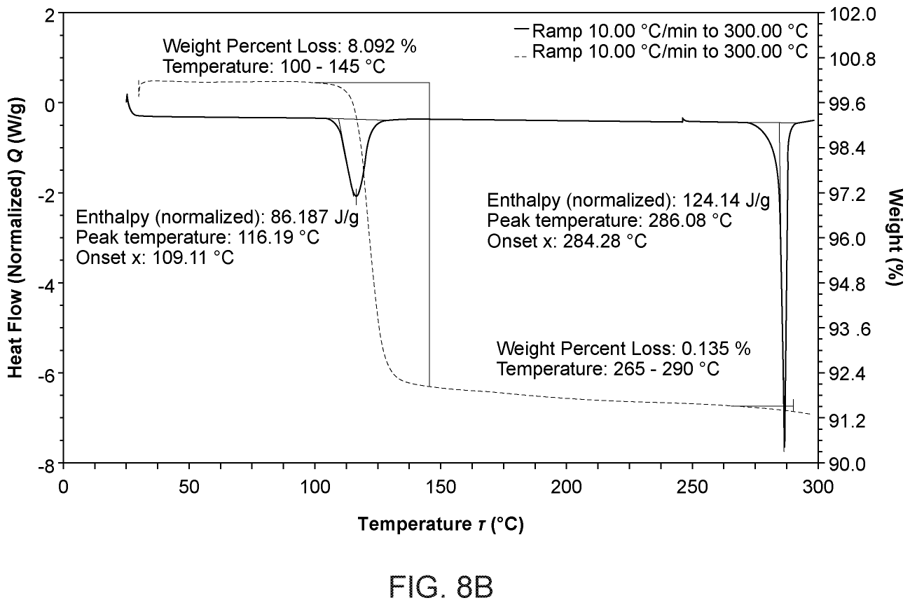

In some embodiments, Compound I Form VIII is further characterized by:
i) one or more peaks at (±0.2°) at 12.2, 16.5, or 20.4 °2θ;
ii) a diffractogram substantially as shown in FIG. 8A;
iii) a differential scanning calorimetry (DSC) curve comprising an endotherm at about 109° C. (onset temperature) and an endotherm at about 284° C. (onset temperature);
iv) a differential scanning calorimetry (DSC) curve substantially as shown in FIG. 8B;
v) thermogravimetric analysis (TGA) showing a weight loss of about 8.2 wt % up to about 290° C.; or
vi) thermogravimetric analysis (TGA) comprising a thermogram substantially as shown in FIG. 8B.

In some embodiments, Compound I Form VIII is further characterized by an X-ray powder diffractogram comprising one or more peaks at (±0.2°) at 12.2, 16.5, or 20.4 °2θ. In some embodiments, Compound I Form VIII is further characterized by an X-ray powder diffractogram comprising one or more peaks at (±0.2°) at 12.7, 19.5, or 22.1 °2θ as determined on a diffractometer using Cu-Kα radiation. In some embodiments, Compound I Form VIII is further characterized by a diffractogram substantially as shown in FIG. 8A.

In some embodiments, Compound I Form VIII is further characterized by a DSC curve comprising an endotherm at about 109° C. (onset temperature) and an endotherm at about 284° C. (onset temperature). In some embodiments, Compound I Form VIII is further characterized by a DSC curve comprising an endotherm at about 116° C. (peak) and an endotherm at about 286° C. (peak). some embodiments, Compound I Form VIII is further characterized by a DSC curve substantially as shown in FIG. 8B.

In some embodiments, Compound I Form VIII is further characterized by TGA showing a weight loss of about 8.2 wt % up to about 290° C. In some embodiments, Compound I Form VIII is further characterized by TGA comprising a thermogram substantially as shown in FIG. 8B.

In some embodiments, Compound I Form VIII is a solvate. In some embodiments, Compound I Form VIII is an acetic acid solvate.

### Compound I Form IX

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form IX (Compound I Form IX) characterized by an X-ray powder diffractogram comprising peaks (±0.2°) at 8.2, 9.9, and 16.6 °2θ as determined on a diffractometer using Cu-Kα radiation.

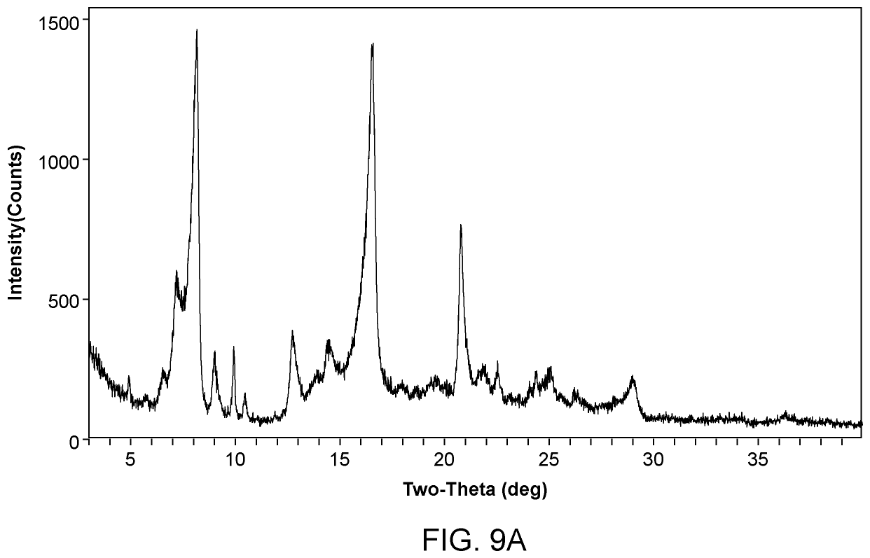

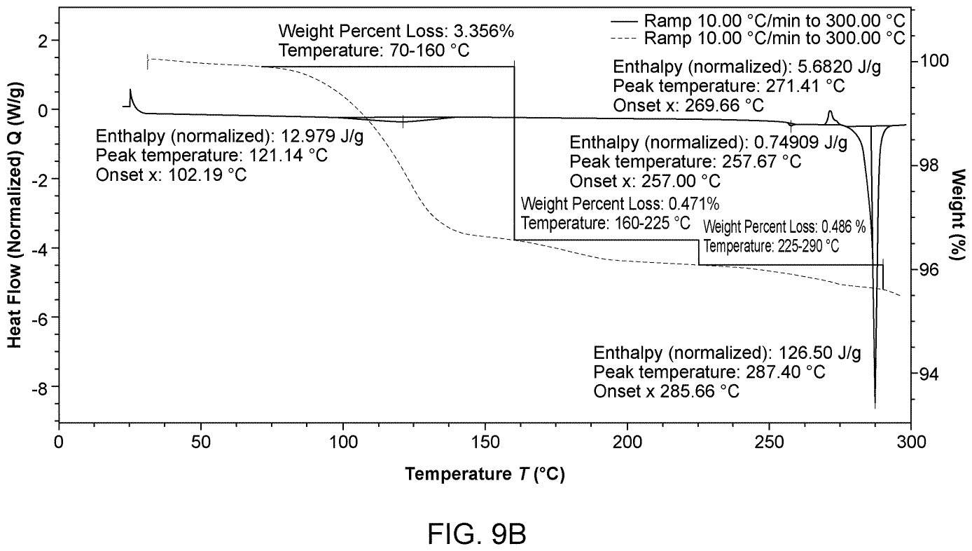

In some embodiments, Compound I Form IX is further characterized by:
i) one or more peaks at (±0.2°) at 9.0, 12.7, or 20.8 °2θ;
ii) a diffractogram substantially as shown in FIG. 9A;
iii) a differential scanning calorimetry (DSC) curve comprising an endotherm at about 102° C. (onset temperature), an endotherm at about 257° C. (onset temperature), and an endotherm at about 286° C. (onset temperature);
iv) a differential scanning calorimetry (DSC) curve substantially as shown in FIG. 9B;
v) thermogravimetric analysis (TGA) showing a weight loss of about 4.4 wt % up to about 290° C.; or
vi) thermogravimetric analysis (TGA) comprising a thermogram substantially as shown in FIG. 9B.

In some embodiments, Compound I Form IX is further characterized by an X-ray powder diffractogram comprising one or more peaks at (±0.2°) at 9.0, 12.7, or 20.8 °2θ. In some embodiments, Compound I Form IX is further characterized by an X-ray powder diffractogram comprising one or more peaks at (±0.2°) at 10.5, 14.4, or 22.5 °2θ as determined on a diffractometer using Cu-Kα radiation. In some embodiments, Compound I Form IX is further characterized by a diffractogram substantially as shown in FIG. 9A.

In some embodiments, Compound I Form IX is further characterized by a DSC curve comprising an endotherm at about 102° C. (onset temperature), an endotherm at about 257° C. (onset temperature), and an endotherm at about 286° C. (onset temperature). In some embodiments, Compound I Form IX is further characterized by a DSC curve comprising an endotherm at about 102° C. (onset temperature), an endotherm at about 257° C. (onset temperature), an exotherm at about 270° C. (onset temperature), and an endotherm at about 286° C. (onset temperature). In some embodiments, Compound I Form IX is further characterized by a DSC curve comprising an endotherm at about 121° C. (peak), an endotherm at about 258° C. (peak), an exotherm at about 271° C. (peak), and an endotherm at about 287° C. (peak). In some embodiments, Compound I Form IX is further characterized by a DSC curve substantially as shown in FIG. 9B.

In some embodiments, Compound I Form IX is further characterized by TGA showing a weight loss of about 4.4 wt % up to about 290° C. In some embodiments, Compound I Form IX is further characterized by TGA comprising a thermogram substantially as shown in FIG. 9B.

In some embodiments, Compound I Form IX is a solvate. In some embodiments, Compound I Form IX is an acetic acid solvate.

### Compound I Form X

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form X (Compound I Form X) characterized by an X-ray powder diffractogram comprising peaks (±0.2°) at 8.1, 9.1, and 18.6 °2θ as determined on a diffractometer using Cu-Kα radiation.

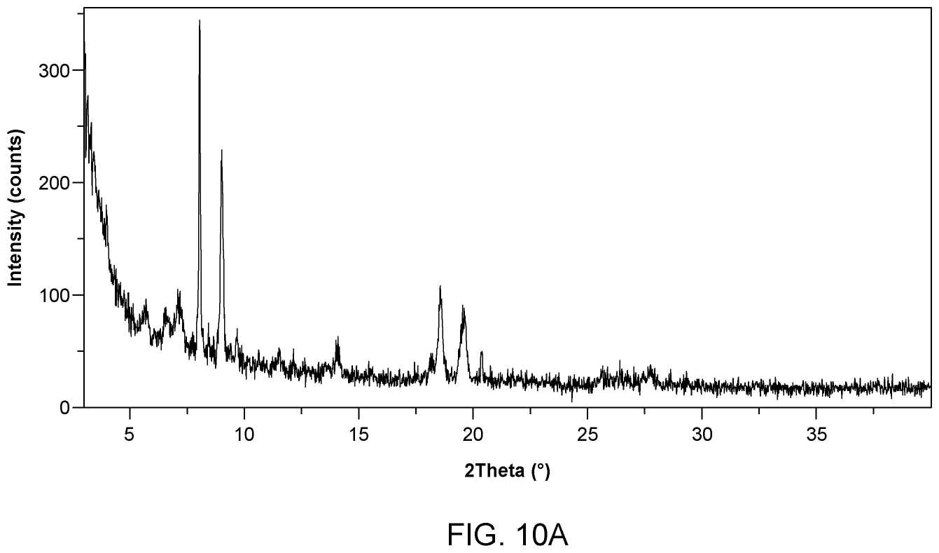

In some embodiments, Compound I Form X is further characterized by:
i) one or more peaks at (±0.2°) at 7.2, 14.1, or 19.6 °2θ; or
ii) a diffractogram substantially as shown in FIG. 10A.

In some embodiments, Compound I Form X is further characterized by an X-ray powder diffractogram comprising one or more peaks at (±0.2°) at 14.1 or 19.6 °2θ. In some embodiments, Compound I Form X is further characterized by a diffractogram substantially as shown in FIG. 10A.

### Compound I Form XI

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form XI (Compound I Form XI) characterized by an X-ray powder diffractogram comprising peaks (±0.2°) at 6.6, 8.1, and 10.4 °2θ as determined on a diffractometer using Cu-Kα radiation.

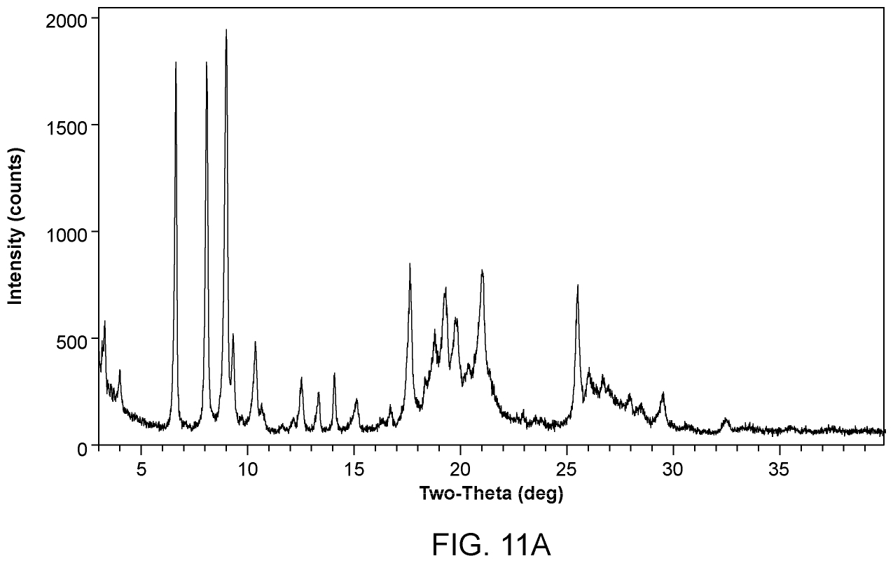

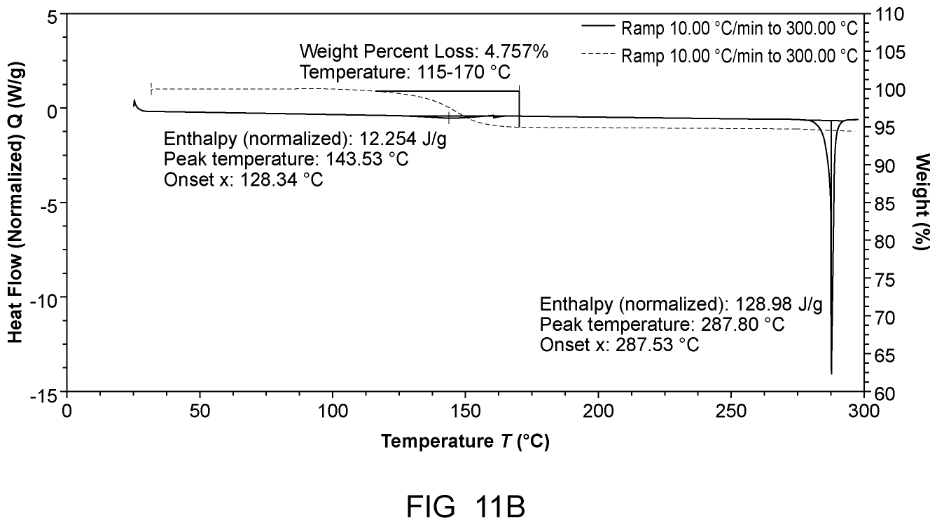

In some embodiments, Compound I Form XI is further characterized by:
i) one or more peaks at (±0.2°) at 9.0, 12.6, or 14.1 °2θ;
ii) a diffractogram substantially as shown in FIG. 11A;
iii) a differential scanning calorimetry (DSC) curve comprising an endotherm at about 128° C. (onset temperature) and an endotherm at about 287° C. (onset temperature);
iv) a differential scanning calorimetry (DSC) curve substantially as shown in FIG. 11B;
v) thermogravimetric analysis (TGA) showing a weight loss of about 4.8 wt % up to about 170° C.; or
vi) thermogravimetric analysis (TGA) comprising a thermogram substantially as shown in FIG. 11B.

In some embodiments, Compound I Form XI is further characterized by an X-ray powder diffractogram comprising one or more peaks at (±0.2°) at 9.0, 12.6, or 14.1 °2θ. In some embodiments, Compound I Form XI is further characterized by an X-ray powder diffractogram comprising one or more peaks at (±0.2°) at 13.4, 17.6, or 19.3 °2θ as determined on a diffractometer using Cu-Kα radiation. In some embodiments, Compound I Form XI is further characterized by a diffractogram substantially as shown in FIG. 11A.

In some embodiments, Compound I Form XI is further characterized by a DSC curve comprising an endotherm at about 128° C. (onset temperature) and an endotherm at about 287° C. (onset temperature). In some embodiments, Compound I Form XI is further characterized by a DSC curve comprising an endotherm at about 144° C. (peak) and an endotherm at about 288° C. (peak). In some embodiments, Compound I Form XI is further characterized by a DSC curve substantially as shown in FIG. 11B.

In some embodiments, Compound I Form XI is further characterized by TGA showing a weight loss of about 4.8 wt % up to about 170° C. In some embodiments, Compound I Form XI is further characterized by TGA comprising a thermogram substantially as shown in FIG. 11B.

In some embodiments, Compound I Form XI is a solvate. In some embodiments, Compound I Form XI is an isopropanol solvate.

### Compound I Form XII

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form XII (Compound I Form XII) characterized by an X-ray powder diffractogram comprising peaks (±0.2°) at 5.8, 8.1, and 9.1 °2θ as determined on a diffractometer using Cu-Kα radiation.

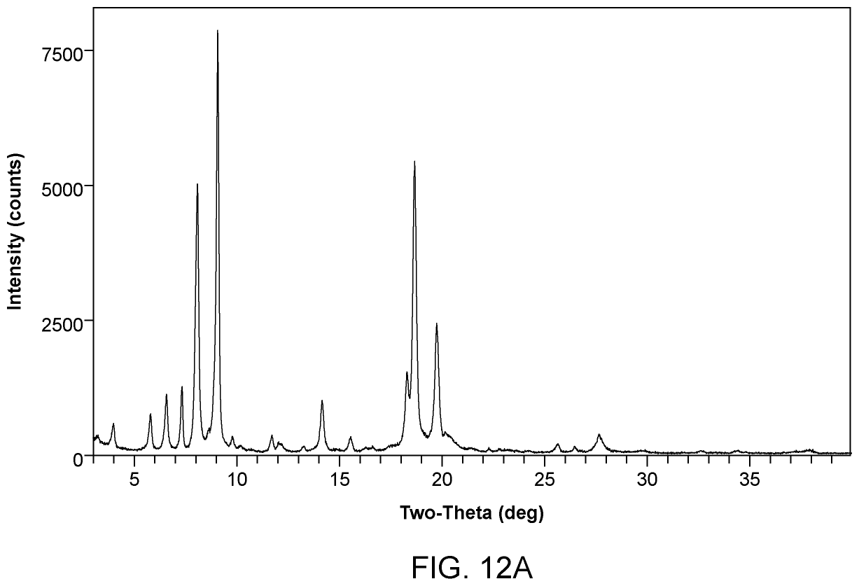

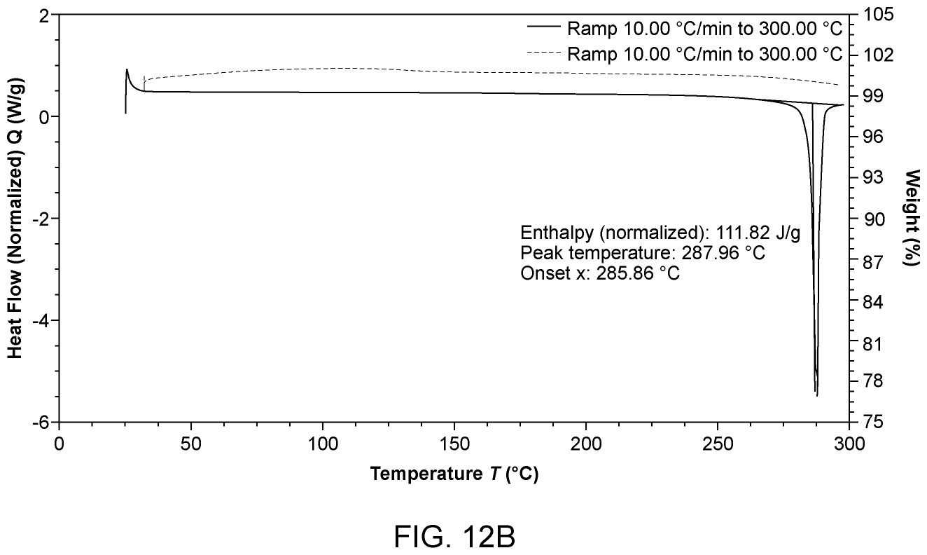

In some embodiments, Compound I Form XII is further characterized by:
i) one or more peaks at (±0.2°) at 6.6, 7.3, or 14.2 °2θ;
ii) a diffractogram substantially as shown in FIG. 12A;
iii) a differential scanning calorimetry (DSC) curve comprising an endotherm at about 286° C. (onset temperature);
iv) a differential scanning calorimetry (DSC) curve substantially as shown in FIG. 12B;
v) thermogravimetric analysis (TGA) showing negligible weight loss; or
vi) thermogravimetric analysis (TGA) comprising a thermogram substantially as shown in FIG. 12B.

In some embodiments, Compound I Form XII is further characterized by an X-ray powder diffractogram comprising one or more peaks at (±0.2°) at 6.6, 7.3, or 14.2 °2θ. In some embodiments, Compound I Form XII is further characterized by an X-ray powder diffractogram comprising one or more peaks at (±0.2°) at 4.0, 18.7, or 19.8 °2θ as determined on a diffractometer using Cu-Kα radiation. In some embodiments, Compound I Form XII is further characterized by a diffractogram substantially as shown in FIG. 12A.

In some embodiments, Compound I Form XII is further characterized by a DSC curve comprising an endotherm at about 286° C. (onset temperature). In some embodiments, Compound I Form XII is further characterized by a DSC curve comprising an endotherm at about 288° C. (peak). In some embodiments, Compound I Form XII is further characterized by a DSC curve substantially as shown in FIG. 12B.

In some embodiments, Compound I Form XII is further characterized by TGA showing negligible weight loss. In some embodiments, Compound I Form XII is further characterized by TGA comprising a thermogram substantially as shown in FIG. 12B.

### Compound I Form XIII

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form XIII (Compound I Form XIII) characterized by an X-ray powder diffractogram comprising peaks (±0.2°) at 6.6, 8.9, and 13.3 °2θ as determined on a diffractometer using Cu-Kα radiation.

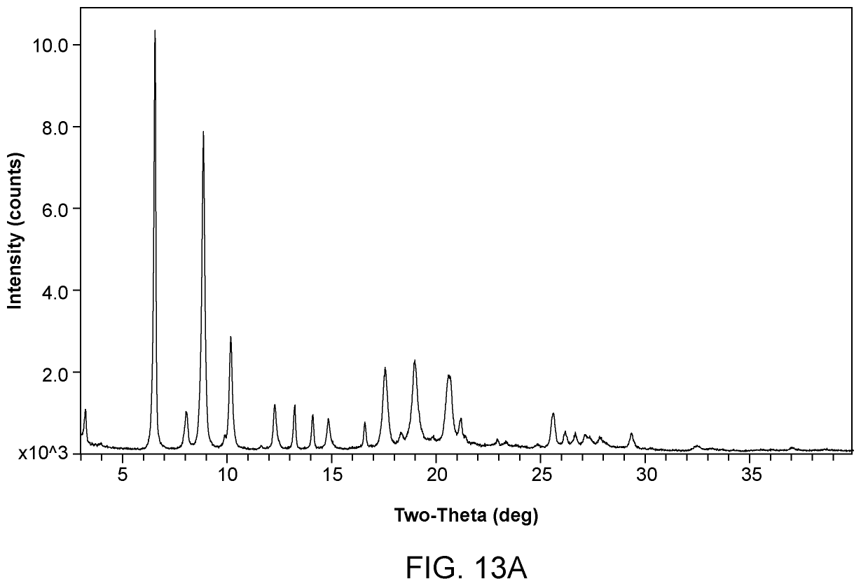

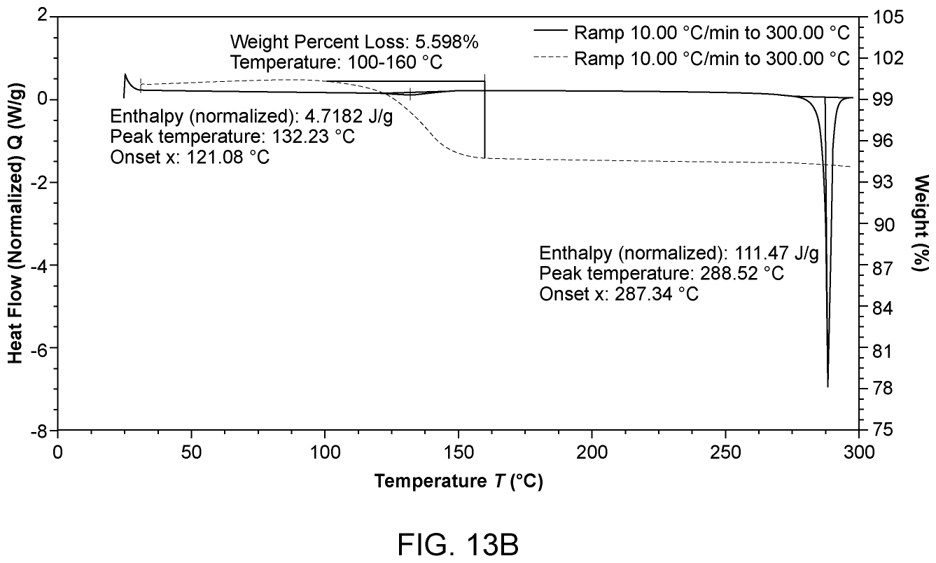

In some embodiments, Compound I Form XIII is further characterized by:
i) one or more peaks at (±0.2°) at 10.2, 12.3, or 17.6 °2θ;
ii) a diffractogram substantially as shown in FIG. 13A;
iii) a differential scanning calorimetry (DSC) curve comprising an endotherm at about 121° C. (onset temperature) and an endotherm at about 287° C. (onset temperature);
iv) a differential scanning calorimetry (DSC) curve substantially as shown in FIG. 13B;
v) thermogravimetric analysis (TGA) showing a weight loss of about 5.6 wt % up to about 160° C.; or
vi) thermogravimetric analysis (TGA) comprising a thermogram substantially as shown in FIG. 13B.

In some embodiments, Compound I Form XIII is further characterized by an X-ray powder diffractogram comprising one or more peaks at (±0.2°) at 10.2, 12.3, or 17.6 °2θ. In some embodiments, Compound I Form XIII is further characterized by an X-ray powder diffractogram comprising one or more peaks at (±0.2°) at 8.1, 14.1, or 19.0 °2θ as determined on a diffractometer using Cu-Kα radiation. In some embodiments, Compound I Form XIII is further characterized by a diffractogram substantially as shown in FIG. 13A.

In some embodiments, Compound I Form XIII is further characterized by a DSC curve comprising an endotherm at about 121° C. (onset temperature) and an endotherm at about 287° C. (onset temperature). In some embodiments, Compound I Form XIII is further characterized by a DSC curve comprising an endotherm at about 132° C. (peak) and an endotherm at about 289° C. (peak). In some embodiments, Compound I Form XIII is further characterized by a DSC curve substantially as shown in FIG. 13B.

In some embodiments, Compound I Form XIII is further characterized by TGA showing a weight loss of about 5.6 wt % up to about 160° C. In some embodiments, Compound I Form XIII is further characterized by TGA comprising a thermogram substantially as shown in FIG. 13B.

In some embodiments, Compound I Form XIII is a solvate. In some embodiments, Compound I Form XIII is a methyl isobutyl ketone (MIBK) solvate.

### Compound I Form XIV

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide Form XIV (Compound I Form XIV) characterized by an X-ray powder diffractogram comprising peaks (±0.2°) at 6.5, 8.8, and 18.2 °2θ as determined on a diffractometer using Cu-Kα radiation.

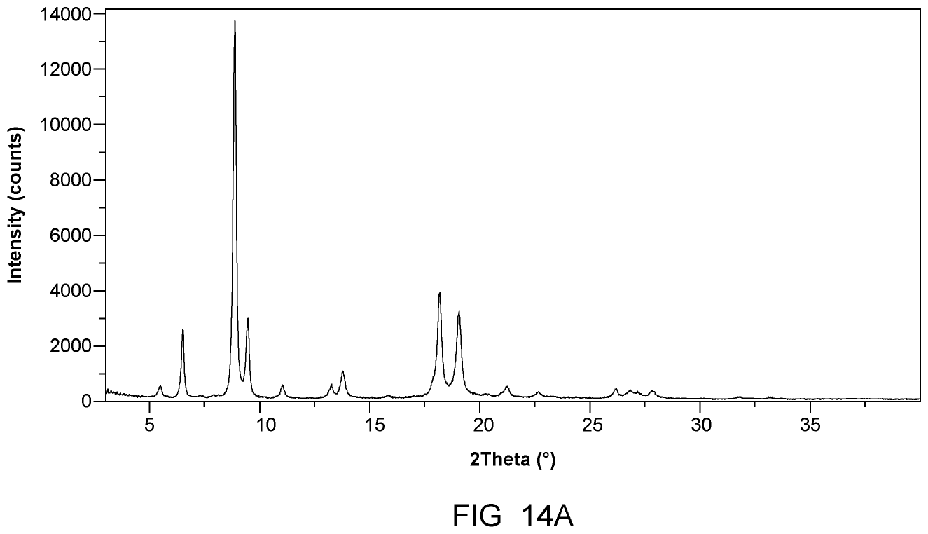

In some embodiments, Compound I Form XIV is further characterized by:
i) one or more peaks at (±0.2°) at 9.4, 13.8, or 19.0 °2θ;
ii) a diffractogram substantially as shown in FIG. 14A.

In some embodiments, Compound I Form XIV is further characterized by an X-ray powder diffractogram comprising one or more peaks at (±0.2°) at 9.4, 13.8, or 19.0 °2θ. In some embodiments, Compound I Form XIV is further characterized by an X-ray powder diffractogram comprising one or more peaks at (±0.2°) at 5.5, 11.0, or 13.2 °2θ as determined on a diffractometer using Cu-Kα radiation. In some embodiments, Compound I Form XIV is further characterized by a diffractogram substantially as shown in FIG. 14A.

### Compound I Phosphate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form I (Compound I Phosphate Form I) characterized by an X-ray powder diffractogram comprising peaks (±0.2°) at 9.1, 14.6, and 18.2 °2θ as determined on a diffractometer using Cu-Kα radiation.

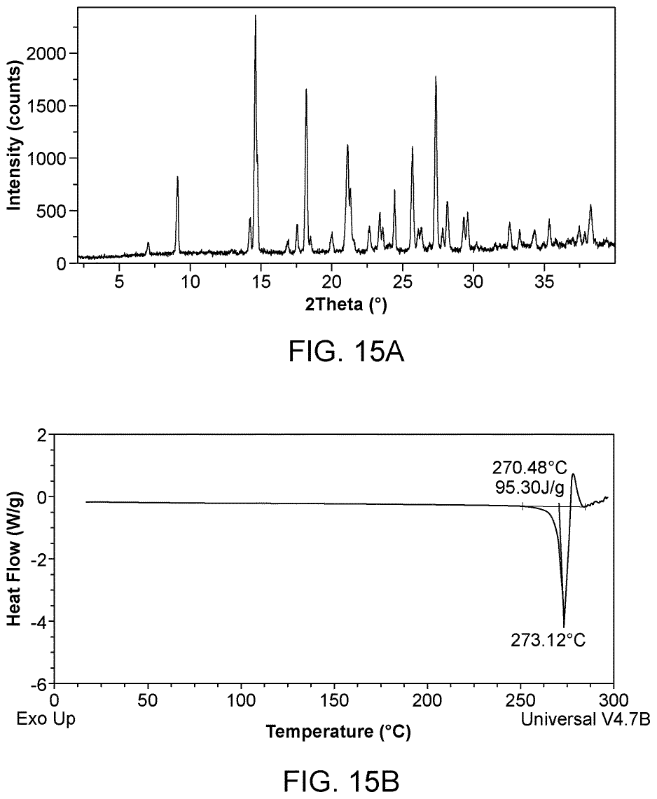

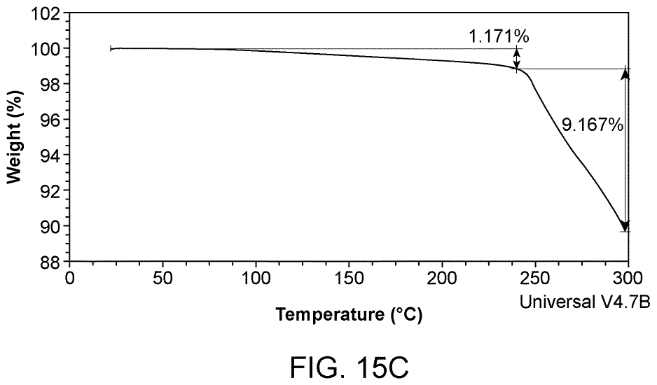

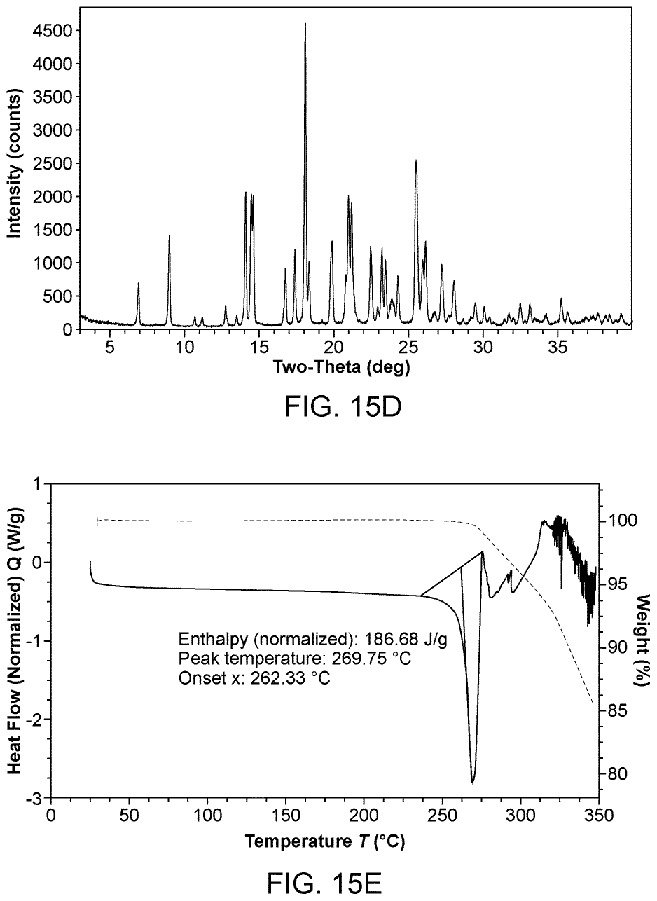

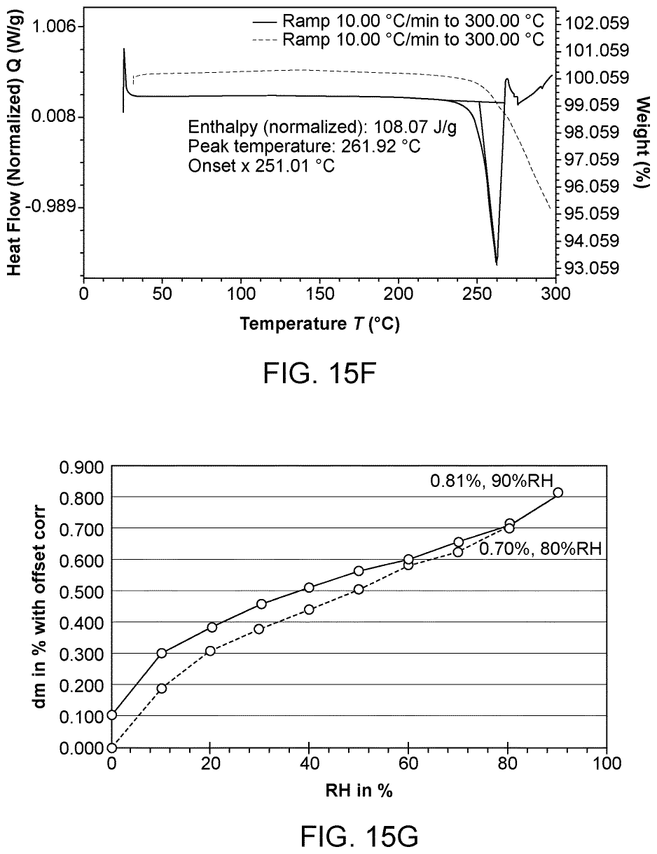

In some embodiments, Compound I Phosphate Form I is further characterized by:
i) one or more peaks at (±0.2°) at 7.1, 14.3, or 17.6 °2θ;
ii) a diffractogram substantially as shown in FIG. 15A;
iii) a differential scanning calorimetry (DSC) curve comprising an endotherm at about 270° C. (onset temperature);
iv) a differential scanning calorimetry (DSC) curve substantially as shown in FIG. 15B;
v) thermogravimetric analysis (TGA) showing a weight loss of about 1.2 wt % up to about 240° C.; or
vi) thermogravimetric analysis (TGA) comprising a thermogram substantially as shown in FIG. 15C.

In some embodiments, Compound I Phosphate Form I is further characterized by an X-ray powder diffractogram comprising one or more peaks at (±0.2°) at one or more peaks at (±0.2°) at 7.1, 14.3, or 17.6 °2θ. In some embodiments, Compound I Phosphate Form I is further characterized by an X-ray powder diffractogram comprising one or more peaks at (±0.2°) at 16.9, 21.1, or 24.5 °2θ as determined on a diffractometer using Cu-Kα radiation. In some embodiments, Compound I Phosphate Form I is further characterized by a diffractogram substantially as shown in FIG. 15A.

In some embodiments, Compound I Phosphate Form I is further characterized by a DSC curve comprising an endotherm at about 270° C. (onset temperature). In some embodiments, Compound I Phosphate Form I is further characterized by a DSC curve comprising an endotherm at about 273° C. (peak). In some embodiments, Compound I Phosphate Form I is further characterized by a DSC curve substantially as shown in FIG. 15B.

In some embodiments, Compound I Phosphate Form I is further characterized by TGA showing a weight loss of about 1.2 wt % up to about 240° C. In some embodiments, Compound I Phosphate Form I is further characterized by TGA comprising a thermogram substantially as shown in FIG. 15C.

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form I (Compound I Phosphate Form I) characterized by an X-ray powder diffractogram comprising peaks (±0.2°) at 6.9, 8.9, and 14.4 °2θ as determined on a diffractometer using Cu-Kα radiation.

In some embodiments, Compound I Phosphate Form I is further characterized by:
i) one or more peaks at (±0.2°) at 14.1, 16.7, or 18.0 °2θ;
ii) a diffractogram substantially as shown in FIG. 15D;
iii) a differential scanning calorimetry (DSC) curve comprising an endotherm at about 262° C. (onset temperature);
iv) a differential scanning calorimetry (DSC) curve substantially as shown in FIG. 15E;
v) a differential scanning calorimetry (DSC) curve comprising an endotherm at about 251° C. (onset temperature);
vi) a differential scanning calorimetry (DSC) curve substantially as shown in FIG. 15F;
vii) thermogravimetric analysis (TGA) showing no weight loss up to about 200° C.;
viii) thermogravimetric analysis (TGA) comprising a thermogram substantially as shown in FIG. 15E or FIG. 15F;
ix) a dynamic vapor sorption (DVS) curve showing about 0.70% water uptake at 80% relative humidity (RH) or about 0.81% water uptake at 90% RH; or
x) a DVS curve substantially as shown in FIG. 15G.

In some embodiments, Compound I Phosphate Form I is further characterized by an X-ray powder diffractogram comprising one or more peaks at (±0.2°) at 14.1, 16.7, or 18.0 °2θ. In some embodiments, Compound I Phosphate Form I is further characterized by an X-ray powder diffractogram comprising one or more peaks at (±0.2°) at 19.8, 21.1, or 23.4 °2θ as determined on a diffractometer using Cu-Kα radiation. In some embodiments, Compound I Phosphate Form I is further characterized by a diffractogram substantially as shown in FIG. 15D.

In some embodiments, Compound I Phosphate Form I is further characterized by a DSC curve comprising an endotherm at about 262° C. (onset temperature). In some embodiments, Compound I Phosphate Form I is further characterized by a DSC curve comprising an endotherm at about 270° C. (peak). In some embodiments, Compound I Phosphate Form I is further characterized by a DSC curve substantially as shown in FIG. 15E.

In some embodiments, Compound I Phosphate Form I is further characterized by a DSC curve comprising an endotherm at about 251° C. (onset temperature). In some embodiments, Compound I Phosphate Form I is further characterized by a DSC curve comprising an endotherm at about 262° C. (peak). In some embodiments, Compound I Phosphate Form I is further characterized by a DSC curve substantially as shown in FIG. 15F.

In some embodiments, Compound I Phosphate Form I is further characterized by TGA showing no weight loss up to about 200° C. In some embodiments, Compound I Phosphate Form I is further characterized by TGA comprising a thermogram substantially as shown in FIG. 15E or FIG. 15F. In some embodiments, Compound I Phosphate Form I is further characterized by TGA comprising a thermogram substantially as shown in FIG. 15E. In some embodiments, Compound I Phosphate Form I is further characterized by TGA comprising a thermogram substantially as shown in FIG. 15F.

In some embodiments, Compound I Phosphate Form I is further characterized by a DVS curve showing about 0.70% water uptake at 80% relative humidity (RH) or about 0.81% water uptake at 90% RH. In some embodiments, Compound I Phosphate Form I is further characterized by a DVS curve substantially as shown in FIG. 15G.

In some embodiments, Compound I Phosphate Form I is an anhydrate.

In some embodiments, Compound I Phosphate Form I has unit cell parameters: a=12.9509(2) A, b=8.22450(10) Å, c=25.4204(3)Å, α=90°, β=100.0490(10°), and γ=90°.

In some embodiments, Compound I Phosphate Form I has unit cell parameters: =12.9509(2) Å, b=8.22450(10) Å, c=25.4204(3)Å, α=90°, β=100.0490(10°), and γ=90° and volume=2666.11(6) 3.

### Compound I Fumarate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide fumarate salt Form I (Compound I Fumarate Form I) characterized by an X-ray powder diffractogram comprising peaks (±0.2°) at 5.5, 7.0, and 19.4 °2θ as determined on a diffractometer using Cu-Kα radiation.

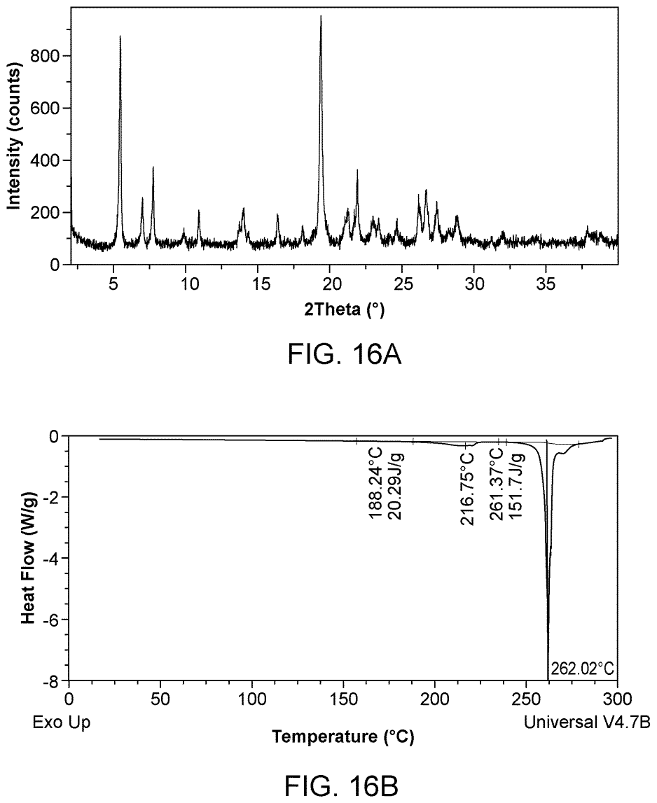

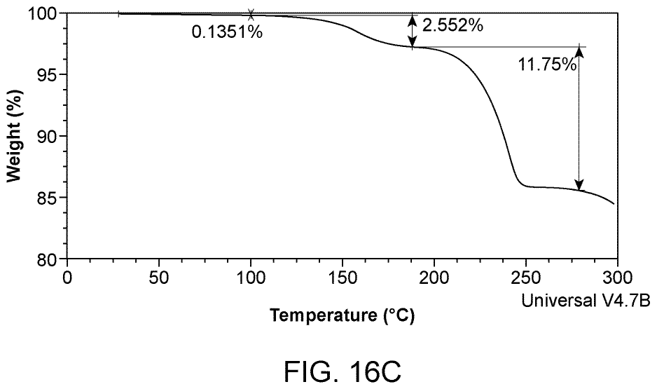

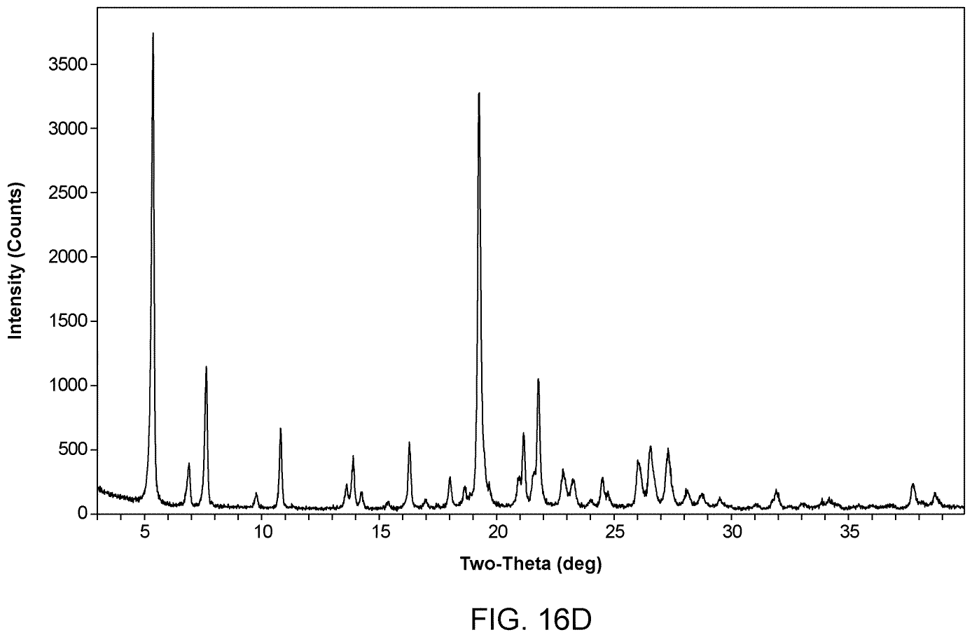

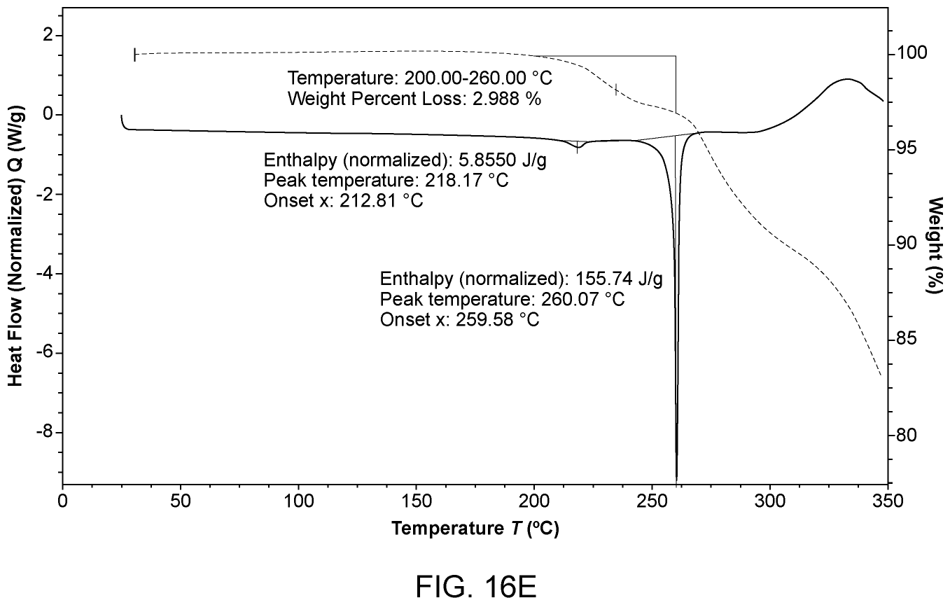

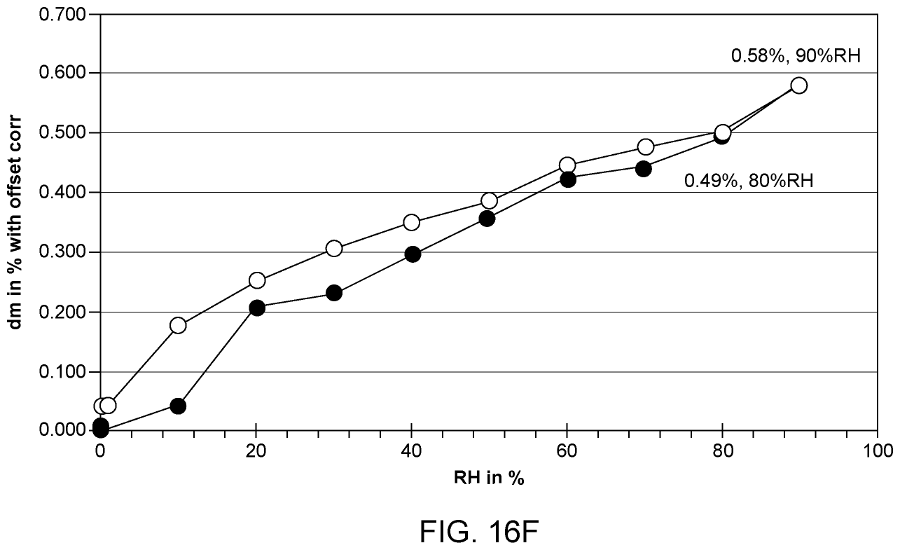

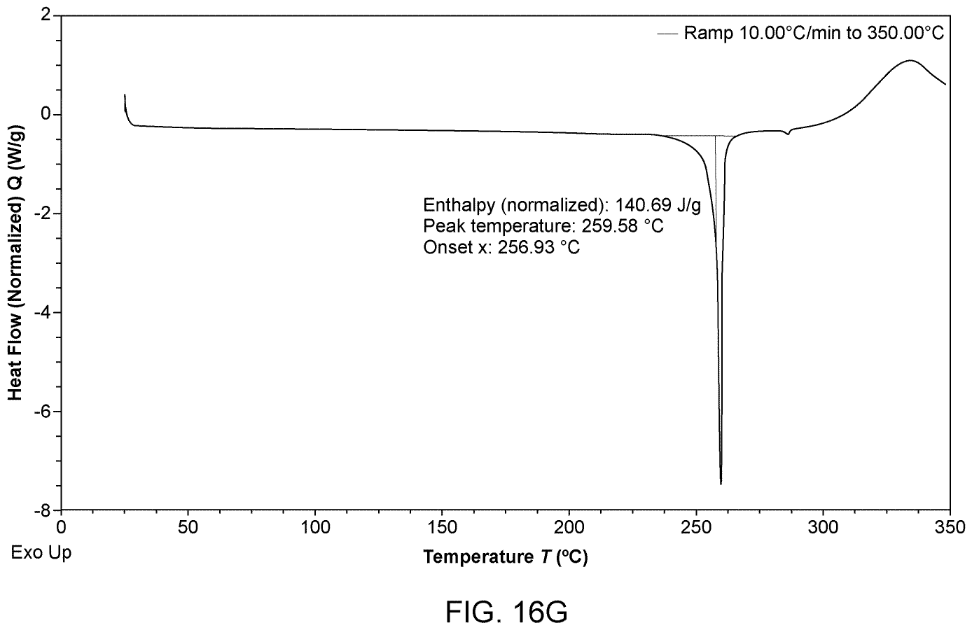

In some embodiments, Compound I Fumarate Form I is further characterized by:
i) one or more peaks at (±0.2°) at 7.7, 10.9, or 16.4 °2θ;
ii) a diffractogram substantially as shown in FIG. 16A;
iii) a differential scanning calorimetry (DSC) curve comprising an endotherm at about 188° C. (onset temperature) and an endotherm at about 261° C. (onset temperature);
iv) a differential scanning calorimetry (DSC) curve substantially as shown in FIG. 16B;
v) thermogravimetric analysis (TGA) showing negligible weight loss up to about 100° C., a weight loss of about 2.6 wt % between about 100° C. to about 188° C., and a weight loss of about 11.8 wt % from about 188° C. to about 279° C.; or
vi) thermogravimetric analysis (TGA) comprising a thermogram substantially as shown in FIG. 16C.

In some embodiments, Compound I Fumarate Form I is further characterized by an X-ray powder diffractogram comprising one or more peaks at (±0.2°) at 7.7, 10.9, or 16.4 °2θ. In some embodiments, Compound I Fumarate Form I is further characterized by an X-ray powder diffractogram comprising one or more peaks at (±0.2°) at 14.0, 18.1, or 21.3 °2θ as determined on a diffractometer using Cu-Kα radiation. In some embodiments, Compound I Fumarate Form I is further characterized by a diffractogram substantially as shown in FIG. 16A.

In some embodiments, Compound I Fumarate Form I is further characterized by a DSC curve comprising an endotherm at about 188° C. (onset temperature) and an endotherm at about 261° C. (onset temperature). In some embodiments, Compound I Fumarate Form I is further characterized by a DSC curve comprising an endotherm at about 217° C. (peak) and an endotherm at about 262° C. (peak). In some embodiments, Compound I Fumarate Form I is further characterized by a DSC curve substantially as shown in FIG. 16B.

In some embodiments, Compound I Fumarate Form I is further characterized by TGA showing negligible weight loss up to about 100° C., a weight loss of about 2.6 wt % between about 100° C. to about 188° C., and an additional weight loss of about 11.8 wt % from about 188° C. to about 279° C. In some embodiments, Compound I Fumarate Form I is further characterized by TGA comprising a thermogram substantially as shown in FIG. 16C.

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide fumarate salt Form I (Compound I Fumarate Form I) characterized by an X-ray powder diffractogram comprising peaks (±0.2°) at 5.4, 6.9, and 19.3 °2θ as determined on a diffractometer using Cu-Kα radiation.

In some embodiments, Compound I Fumarate Form I is further characterized by:
i) one or more peaks at (±0.2°) at 7.6, 10.8, or 16.3 °2θ;
ii) a diffractogram substantially as shown in FIG. 16D;
iii) a differential scanning calorimetry (DSC) curve comprising an endotherm at about 213° C. (onset temperature) and an endotherm at about 260° C. (onset temperature);
iv) a differential scanning calorimetry (DSC) curve substantially as shown in FIG. 16E;
v) thermogravimetric analysis (TGA) showing no weight loss up to about 150° C. and a weight loss of about 3.0 wt % from about 200° C. to about 260° C.;
vi) thermogravimetric analysis (TGA) comprising a thermogram substantially as shown in FIG. 16E;
vii) a dynamic vapor sorption (DVS) curve showing about 0.49% water uptake at 80% relative humidity (RH) or about 0.58% water uptake at 90% RH; or
viii) a DVS curve substantially as shown in FIG. 16F.

In some embodiments, Compound I Fumarate Form I is further characterized by an X-ray powder diffractogram comprising one or more peaks at (±0.2°) at 7.6, 10.8, or 16.3 °2θ. In some embodiments, Compound I Fumarate Form I is further characterized by an X-ray powder diffractogram comprising one or more peaks at (±0.2°) at 13.9, 18.0, or 21.2 °2θ as determined on a diffractometer using Cu-Kα radiation. In some embodiments, Compound I Fumarate Form I is further characterized by a diffractogram substantially as shown in FIG. 16D.

In some embodiments, Compound I Fumarate Form I is further characterized by a DSC curve comprising an endotherm at about 213° C. (onset temperature) and an endotherm at about 260° C. (onset temperature). In some embodiments, Compound I Fumarate Form I is further characterized by a DSC curve comprising an endotherm at about 218° C. (peak) and an endotherm at about 260° C. (peak). In some embodiments, Compound I Fumarate Form I is further characterized by a DSC curve substantially as shown in FIG. 16E.

In some embodiments, Compound I Fumarate Form I is further characterized by TGA showing no weight loss up to about 150° C. and a weight loss of about 3.0 wt % from about 200° C. to about 260° C. In some embodiments, Compound I Fumarate Form I is further characterized by TGA comprising a thermogram substantially as shown in FIG. 16E.

In some embodiments, Compound I Fumarate Form I is further characterized by a DVS curve showing about 0.49% water uptake at 80% relative humidity (RH) or about 0.58% water uptake at 90% RH. In some embodiments, Compound I Fumarate Form I is further characterized by a DVS curve substantially as shown in FIG. 16F.

In some embodiments, Compound I Fumarate Form I is an anhydrate.

### Compound I HCl Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide HCl salt Form I (Compound I HCl Form I) characterized by an X-ray powder diffractogram comprising peaks (±0.2°) at 4.4, 8.9 and 19.7 °2θ as determined on a diffractometer using Cu-Kα radiation.

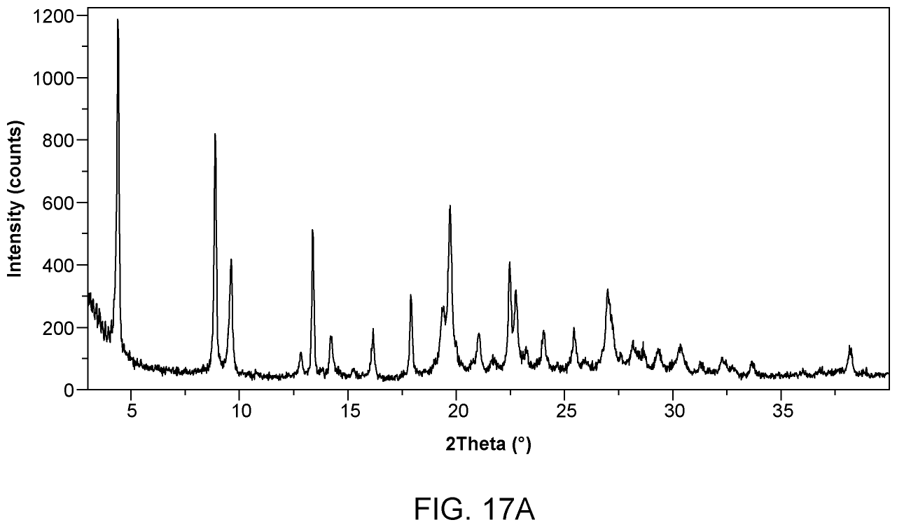

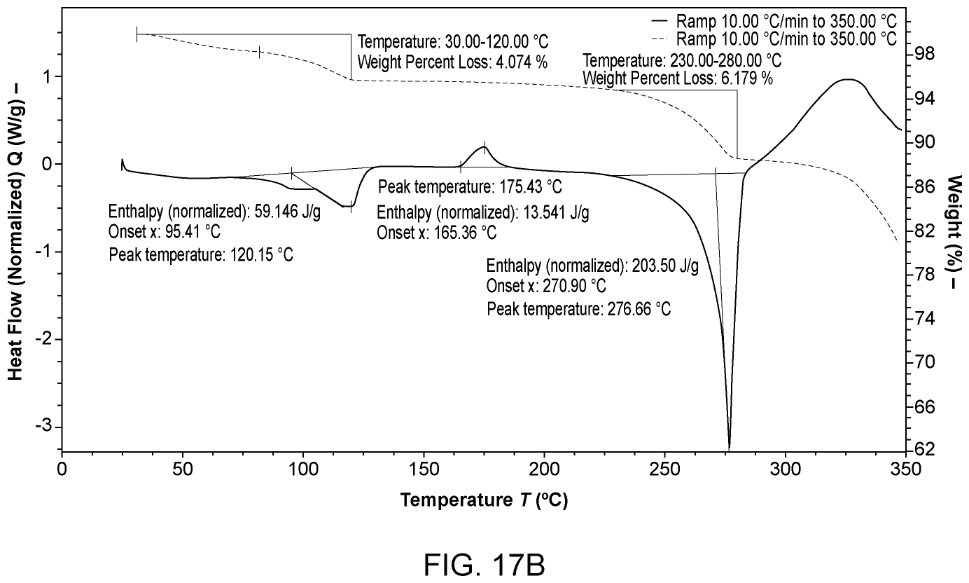

In some embodiments, Compound I HCl Form I is further characterized by:
i) one or more peaks at (±0.2°) at 9.6, 13.4, or 16.2 °2θ;
ii) a diffractogram substantially as shown in FIG. 17A;
iii) a differential scanning calorimetry (DSC) curve comprising an endotherm at about 95° C. (onset temperature) and an endotherm at about 271° C. (onset temperature);
iv) a differential scanning calorimetry (DSC) curve substantially as shown in FIG. 17B;
v) thermogravimetric analysis (TGA) showing about 4.1% weight loss up to about 120° C. and about 6.2% weight loss from about 230° C. to about 280° C.; or
vi) thermogravimetric analysis (TGA) comprising a thermogram substantially as shown in FIG. 17B.

In some embodiments, Compound I HCl Form I is further characterized by an X-ray powder diffractogram comprising one or more peaks at (±0.2°) at 9.6, 13.4, or 16.2 °2θ. In some embodiments, Compound I HCl Form I is further characterized by an X-ray powder diffractogram comprising one or more peaks at (±0.2°) at 12.8, 14.2, or 17.9 °2θ as determined on a diffractometer using Cu-Kα radiation. In some embodiments, Compound I HCl Form I is further characterized by a diffractogram substantially as shown in FIG. 17A.

In some embodiments, Compound I HCl Form I is further characterized by a DSC curve comprising an endotherm at about 95° C. (onset temperature) and an endotherm at about 271° C. (onset temperature). In some embodiments, Compound I HCl Form I is further characterized by a DSC curve comprising an endotherm at about 95° C. (onset temperature), an exotherm at about 165° C. (onset temperature), and an endotherm at about 271° C. (onset temperature). In some embodiments, Compound I HCl Form I is further characterized by a DSC curve comprising an endotherm at about 120° C. (peak), an exotherm at about 175° C. (peak), and an endotherm at about 277° C. (peak). In some embodiments, Compound I HCl Form I is further characterized by a DSC curve substantially as shown in FIG. 17B.

In some embodiments, Compound I HCl Form I is further characterized by TGA showing about 4.1% weight loss up to about 120° C. and about 6.2% weight loss from about 230° C. to about 280° C. In some embodiments, Compound I HCl Form I is further characterized by TGA comprising a thermogram substantially as shown in FIG. 17B.

In some embodiments, Compound I HCl Form I is a hydrate.

### Compound I HCl Form II

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide HCl salt Form II (Compound I HCl Form II) characterized by an X-ray powder diffractogram comprising peaks (±0.2°) at 8.1, 9.3, and 10.9 °2θ as determined on a diffractometer using Cu-Kα radiation.

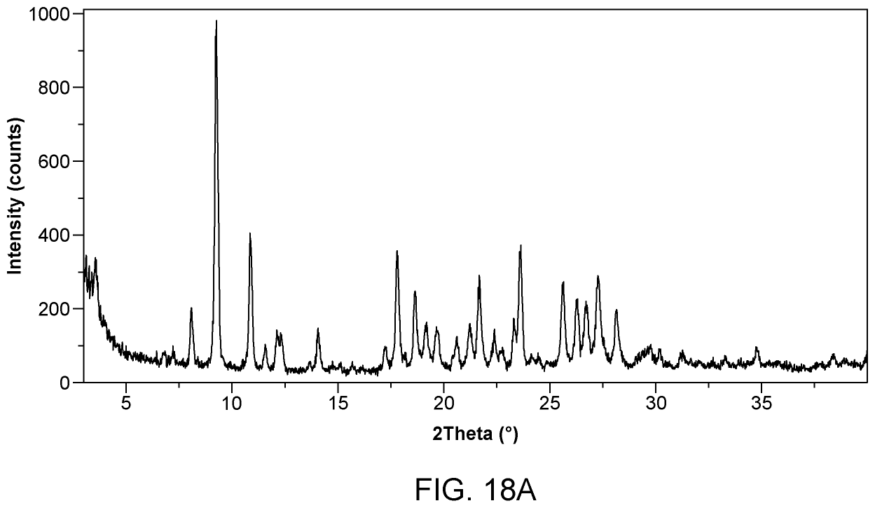

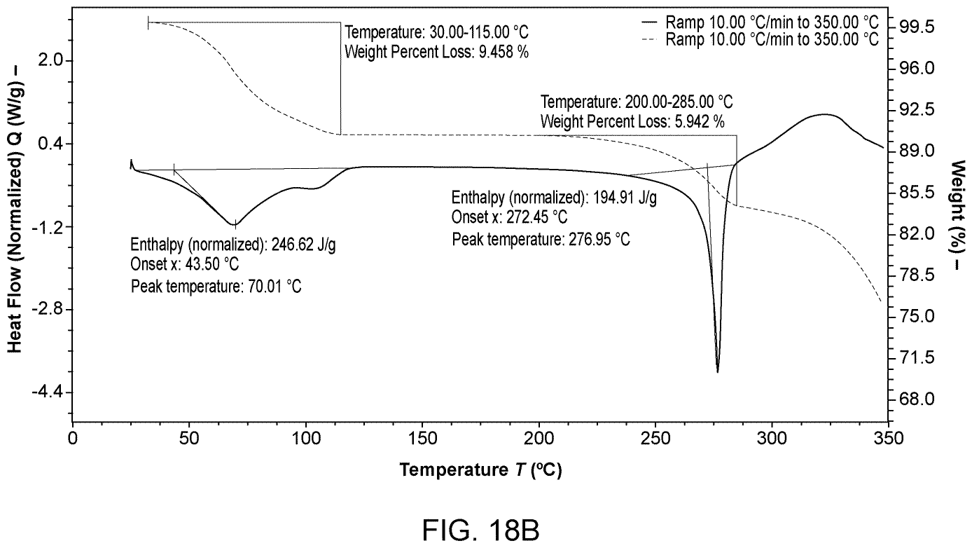

In some embodiments, Compound I HCl Form II is further characterized by:
i) one or more peaks at (±0.2°) at 11.6, 14.1, or 17.8 °2θ;
ii) a diffractogram substantially as shown in FIG. 18A;
iii) a differential scanning calorimetry (DSC) curve comprising an endotherm at about 44° C. (onset temperature) and an endotherm at about 272° C. (onset temperature);
iv) a differential scanning calorimetry (DSC) curve substantially as shown in FIG. 18B;
v) thermogravimetric analysis (TGA) showing about 9.5% weight loss up to about 115° C. and about 5.9% weight loss from about 200° C. to about 285° C.; or
vi) thermogravimetric analysis (TGA) comprising a thermogram substantially as shown in FIG. 18B.

In some embodiments, Compound I HCl Form II is further characterized by an X-ray powder diffractogram comprising one or more peaks at (±0.2°) at 11.6, 14.1, or 17.8 °2θ. In some embodiments, Compound I HCl Form II is further characterized by an X-ray powder diffractogram comprising one or more peaks at (±0.2°) at 18.7, 21.7, or 23.6 °2θ as determined on a diffractometer using Cu-Kα radiation. In some embodiments, Compound I HCl Form II is further characterized by a diffractogram substantially as shown in FIG. 18A.

In some embodiments, Compound I HCl Form II is further characterized by a DSC curve comprising an endotherm at about 44° C. (onset temperature) and an endotherm at about 272° C. (onset temperature). In some embodiments, Compound I HCl Form II is further characterized by a DSC curve comprising an endotherm at about 70° C. (peak) and an endotherm at about 277° C. (peak). In some embodiments, Compound I HCl Form II is further characterized by a DSC curve substantially as shown in FIG. 18B.

In some embodiments, Compound I HCl Form II is further characterized by TGA showing about 9.5% weight loss up to about 115° C. and about 5.9% weight loss from about 200° C. to about 285° C. In some embodiments, Compound I HCl Form II is further characterized by TGA comprising a thermogram substantially as shown in 18B.

In some embodiments, Compound I HCl Form II is a hydrate.

### Compound I Sulfate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide sulfate salt Form I (Compound I Sulfate Form I) characterized by an X-ray powder diffractogram comprising peaks (±0.2°) at 3.6, 7.4, and 17.1 °2θ as determined on a diffractometer using Cu-Kα radiation.

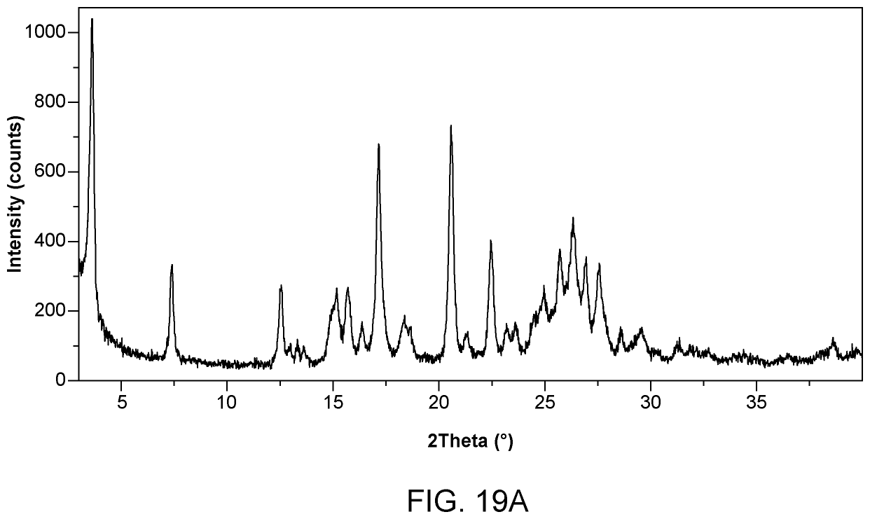

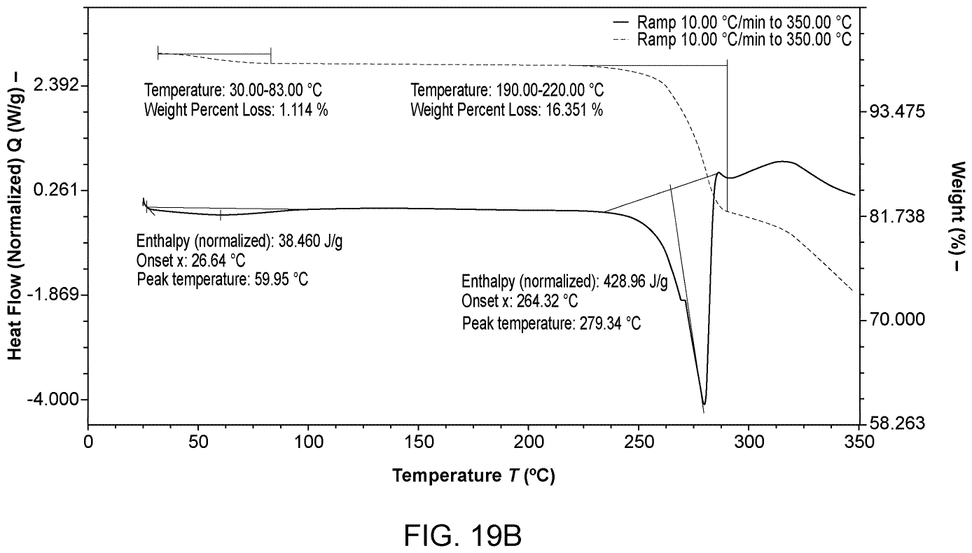

In some embodiments, Compound I Sulfate Form I is further characterized by:
i) one or more peaks at (±0.2°) at 12.6, 15.7, or 20.6 °2θ;
ii) a diffractogram substantially as shown in FIG. 19A;
iii) a differential scanning calorimetry (DSC) curve comprising an endotherm at about 27° C. (onset temperature) and an endotherm at about 264° C. (onset temperature);
iv) a differential scanning calorimetry (DSC) curve substantially as shown in FIG. 19B;
v) thermogravimetric analysis (TGA) showing about 1.1% weight loss up to about 83° C. and about 16.4% weight loss from about 190° C. to about 220° C.; or
vi) thermogravimetric analysis (TGA) comprising a thermogram substantially as shown in FIG. 19B.

In some embodiments, Compound I Sulfate Form I is further characterized by an X-ray powder diffractogram comprising one or more peaks at (±0.2°) at 12.6, 15.7, or 20.6 °2θ. In some embodiments, Compound I Sulfate Form I is further characterized by an X-ray powder diffractogram comprising one or more peaks at (±0.2°) at 16.4, 22.4, or 26.3 °2θ as determined on a diffractometer using Cu-Kα radiation. In some embodiments, Compound I Sulfate Form I is further characterized by a diffractogram substantially as shown in FIG. 19A.

In some embodiments, Compound I Sulfate Form I is further characterized by a DSC curve comprising an endotherm at about 27° C. (onset temperature) and an endotherm at about 264° C. (onset temperature). In some embodiments, Compound I Sulfate Form I is further characterized by a DSC curve comprising an endotherm at about 60° C. (peak) and an endotherm at about 279° C. (peak). In some embodiments, Compound I Sulfate Form I is further characterized by a DSC curve substantially as shown in FIG. 19B.

In some embodiments, Compound I Sulfate Form I is further characterized by a TGA showing about 1.1% weight loss up to about 83° C. and about 16.4% weight loss from about 190° C. to about 220° C. In some embodiments, Compound I Sulfate Form I is further characterized by TGA comprising a thermogram substantially as shown in FIG. 19B.

In some embodiments, Compound I Sulfate Form I is an anhydrate.

### Compound I Maleate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide maleate salt Form I (Compound I Maleate Form I) characterized by an X-ray powder diffractogram comprising peaks (±0.2°) at 5.3, 9.4, and 18.7 °2θ as determined on a diffractometer using Cu-Kα radiation.

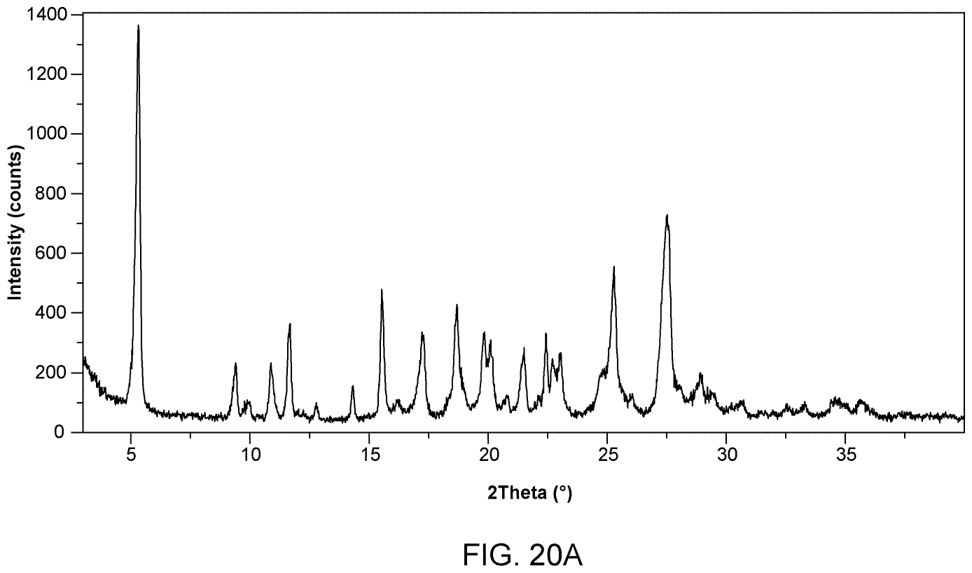

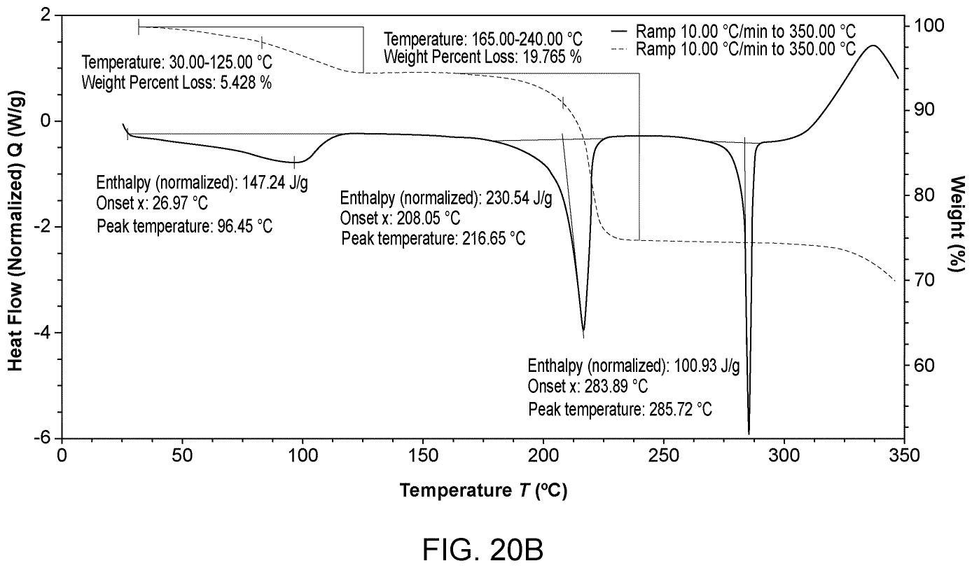

In some embodiments, Compound I Maleate Form I is further characterized by:
i) one or more peaks at (±0.2°) at 10.9, 15.5, or 17.2 °2θ;
ii) a diffractogram substantially as shown in FIG. 20A;
iii) a differential scanning calorimetry (DSC) curve comprising an endotherm at about 27° C. (onset temperature), an endotherm at about 208° C. (onset temperature), and an endotherm at about 284° C. (onset temperature);
iv) a differential scanning calorimetry (DSC) curve substantially as shown in FIG. 20B;
v) thermogravimetric analysis (TGA) showing about 5.4% weight loss up to about 125° C. and about 19.8% weight loss from about 165° C. to about 240° C.; or
vi) thermogravimetric analysis (TGA) comprising a thermogram substantially as shown in FIG. 20B.

In some embodiments, Compound I Maleate Form I is further characterized by an X-ray powder diffractogram comprising one or more peaks at (±0.2°) at 10.9, 15.5, or 17.2 °2θ. In some embodiments, Compound I Maleate Form I is further characterized by an X-ray powder diffractogram comprising one or more peaks at (±0.2°) at 11.7, 14.3, or 21.5 °2θ as determined on a diffractometer using Cu-Kα radiation. In some embodiments, Compound I Maleate Form I is further characterized by a diffractogram substantially as shown in FIG. 20A.

In some embodiments, Compound I Maleate Form I is further characterized by a DSC curve comprising an endotherm at about 27° C. (onset temperature), an endotherm at about 208° C. (onset temperature), and an endotherm at about 284° C. (onset temperature). In some embodiments, Compound I Maleate Form I is further characterized by a DSC curve comprising an endotherm at about 96° C. (peak), an endotherm at about 217° C. (peak), and an endotherm at about 286° C. (peak). In some embodiments, Compound I Maleate Form I is further characterized by a DSC curve substantially as shown in FIG. 20B.

In some embodiments, Compound I Maleate Form I is further characterized by a TGA showing about 5.4% weight loss up to about 125° C. and about 19.8% weight loss from about 165° C. to about 240° C. In some embodiments, Compound I Maleate Form I is further characterized by a TGA comprising a thermogram substantially as shown in FIG. 20B.

In some embodiments, Compound I Maleate Form I is a hydrate.

### Compound I Phosphate Form II

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form II (Compound I Phosphate Form II) characterized by an X-ray powder diffractogram comprising peaks (±0.2°) at 7.3, 9.4, and 14.9 °2θ as determined on a diffractometer using Cu-Kα radiation.

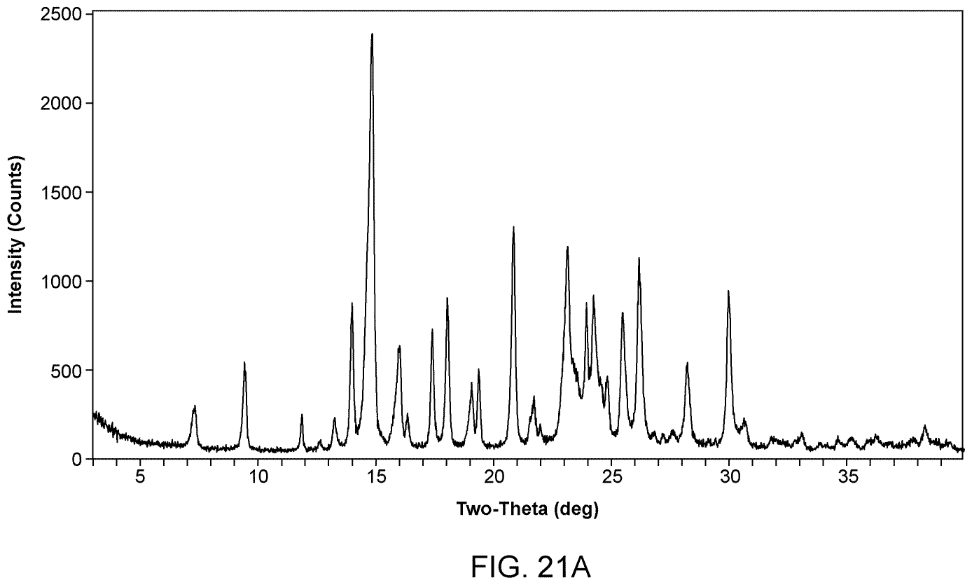

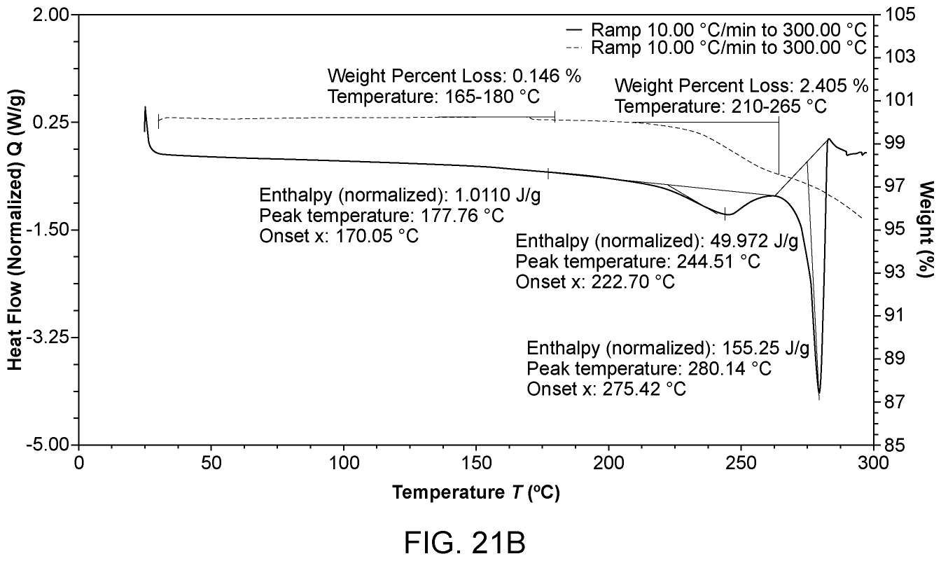

In some embodiments, Compound I Phosphate Form II is further characterized by:
i) one or more peaks at (±0.2°) at 14.0, 16.0, or 18.1 °2θ;
ii) a diffractogram substantially as shown in FIG. 21A;
iii) a differential scanning calorimetry (DSC) curve comprising an endotherm at about 223° C. (onset temperature) and an endotherm at about 275° C. (onset temperature);
iv) a differential scanning calorimetry (DSC) curve substantially as shown in FIG. 21B;
v) thermogravimetric analysis (TGA) showing about 0.1% weight loss up to about 180° C. and about 2.4% weight loss from about 210° C. to about 265° C.; or
vi) thermogravimetric analysis (TGA) comprising a thermogram substantially as shown in FIG. 21B.

In some embodiments, Compound I Phosphate Form II is further characterized by an X-ray powder diffractogram comprising one or more peaks at (±0.2°) at 14.0, 16.0, or 18.1 °2θ. In some embodiments, Compound I Phosphate Form II is further characterized by an X-ray powder diffractogram comprising one or more peaks at (±0.2°) at 17.4, 19.4, or 20.9 °2θ as determined on a diffractometer using Cu-Kα radiation. In some embodiments, Compound I Phosphate Form II is further characterized by a diffractogram substantially as shown in FIG. 21A.

In some embodiments, Compound I Phosphate Form II is further characterized by a DSC curve comprising an endotherm at about 223° C. (onset temperature) and an endotherm at about 275° C. (onset temperature). In some embodiments, Compound I Phosphate Form II is further characterized by a DSC curve comprising an endotherm at about 245° C. (peak), and an endotherm at about 280° C. (peak). In some embodiments, Compound I Phosphate Form II is further characterized by a DSC curve substantially as shown in FIG. 21B.

In some embodiments, Compound I Phosphate Form II is further characterized by a TGA showing about 0.1% weight loss up to about 180° C. and about 2.4% weight loss from about 210° C. to about 265° C. In some embodiments, Compound I Phosphate Form II is further characterized by a TGA comprising a thermogram substantially as shown in FIG. 21B.

In some embodiments, Compound I Phosphate Form II is an anhydrate.

### Compound I Succinate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide succinate salt Form I (Compound I Succinate Form I) characterized by an X-ray powder diffractogram comprising peaks (±0.2°) at 5.3, 10.8, and 19.1 °2θ as determined on a diffractometer using Cu-Kα radiation.

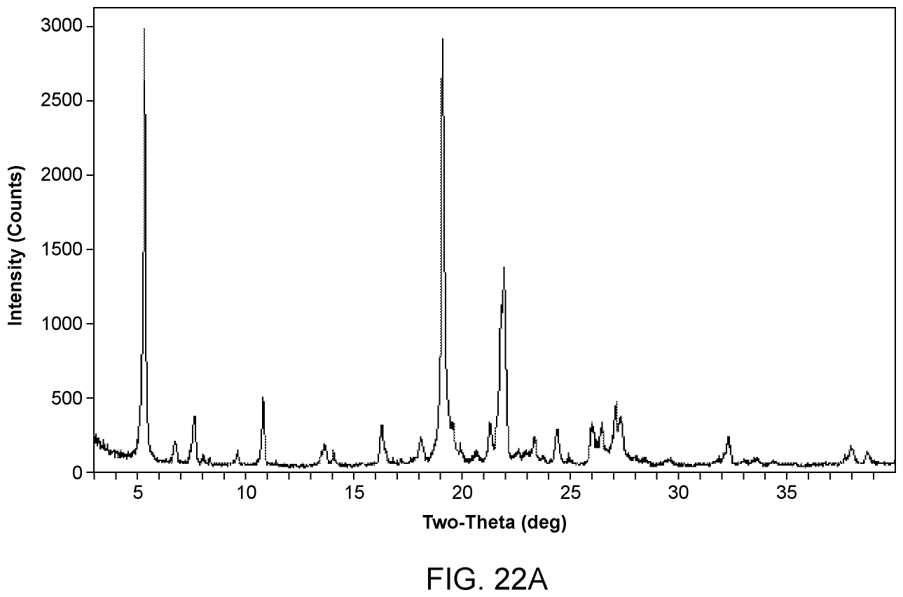

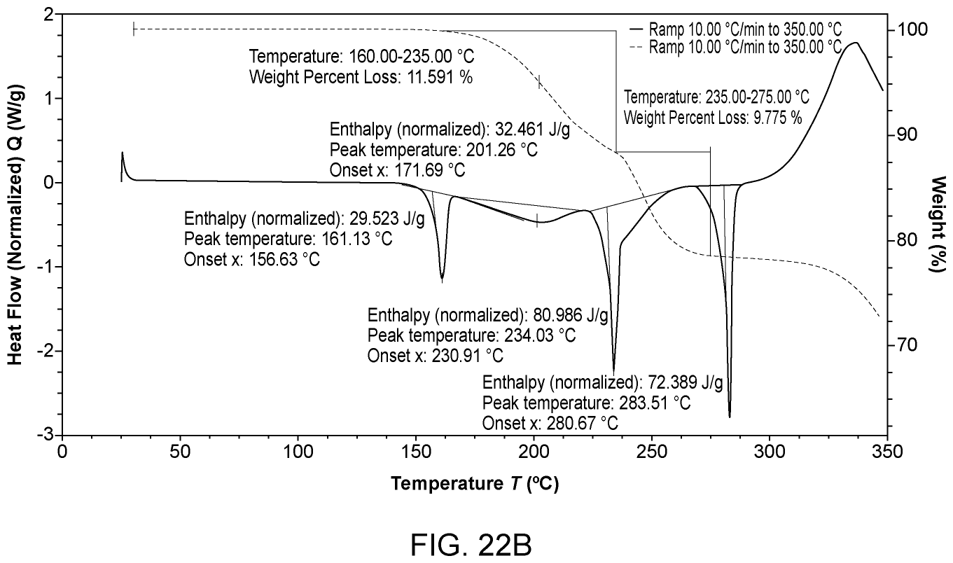

In some embodiments, Compound I Succinate Form I is further characterized by:
i) one or more peaks at (±0.2°) at 7.6, 16.3, or 21.9 °2θ;
ii) a diffractogram substantially as shown in FIG. 22A;
iii) a differential scanning calorimetry (DSC) curve comprising an endotherm at about 157° C. (onset temperature), an endotherm at about 172° C. (onset temperature), an endotherm at about 231° C. (onset temperature), and an endotherm at about 281° C. (onset temperature);
iv) a differential scanning calorimetry (DSC) curve substantially as shown in FIG. 22B;
v) thermogravimetric analysis (TGA) showing about 11.5% weight loss up to about 235° C. and about 9.8% weight loss from about 235° C. to about 275° C.; or
vi) thermogravimetric analysis (TGA) comprising a thermogram substantially as shown in FIG. 22B.

In some embodiments, Compound I Succinate Form I is further characterized by an X-ray powder diffractogram comprising one or more peaks at (±0.2°) at 7.6, 16.3, or 21.9 °2θ. In some embodiments, Compound I Succinate Form I is further characterized by an X-ray powder diffractogram comprising one or more peaks at (±0.2°) at 6.8, 13.7, or 18.0 °2θ as determined on a diffractometer using Cu-Kα radiation. In some embodiments, Compound I Succinate Form I is further characterized by a diffractogram substantially as shown in FIG. 22A.

In some embodiments, Compound I Succinate Form I is further characterized by DSC curve comprising an endotherm at about 157° C. (onset temperature), an endotherm at about 172° C. (onset temperature), an endotherm at about 231° C. (onset temperature), and an endotherm at about 281° C. (onset temperature). In some embodiments, Compound I Succinate Form I is further characterized by a DSC curve comprising an endotherm at about 161° C. (peak), an endotherm at about 201° C. (peak), an endotherm at about 234° C. (peak), and an endotherm at about 284° C. (peak). In some embodiments, Compound I Succinate Form I is further characterized by a DSC curve substantially as shown in FIG. 22B.

In some embodiments, Compound I Succinate Form I is further characterized by a TGA showing about 11.5% weight loss up to about 235° C. and about 9.8% weight loss from about 235° C. to about 275° C. In some embodiments, Compound I Succinate Form I is further characterized by a TGA comprising a thermogram substantially as shown in FIG. 22B.

In some embodiments, Compound I Succinate Form I is an anhydrate.

### Compound I Tosylate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide tosylate salt Form I (Compound I Tosylate Form I) characterized by an X-ray powder diffractogram comprising peaks (±0.2°) at 3.2, 9.9, and 17.8 °2θ as determined on a diffractometer using Cu-Kα radiation.

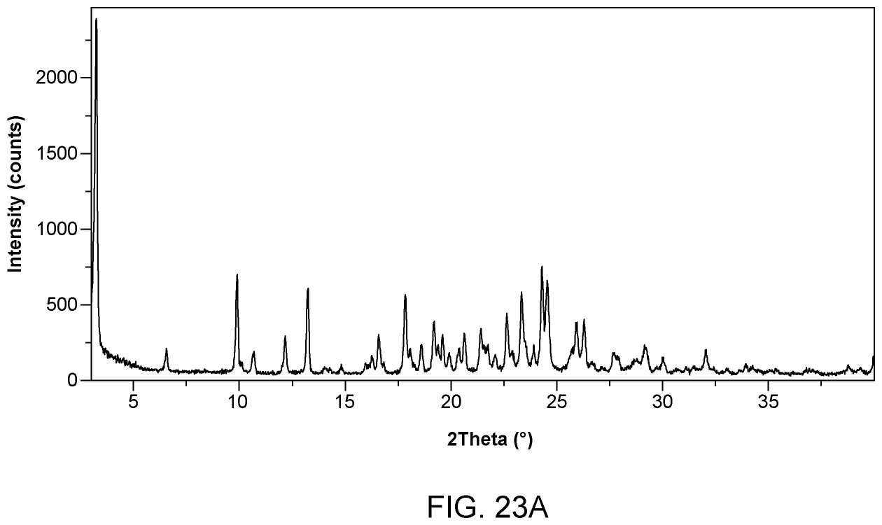

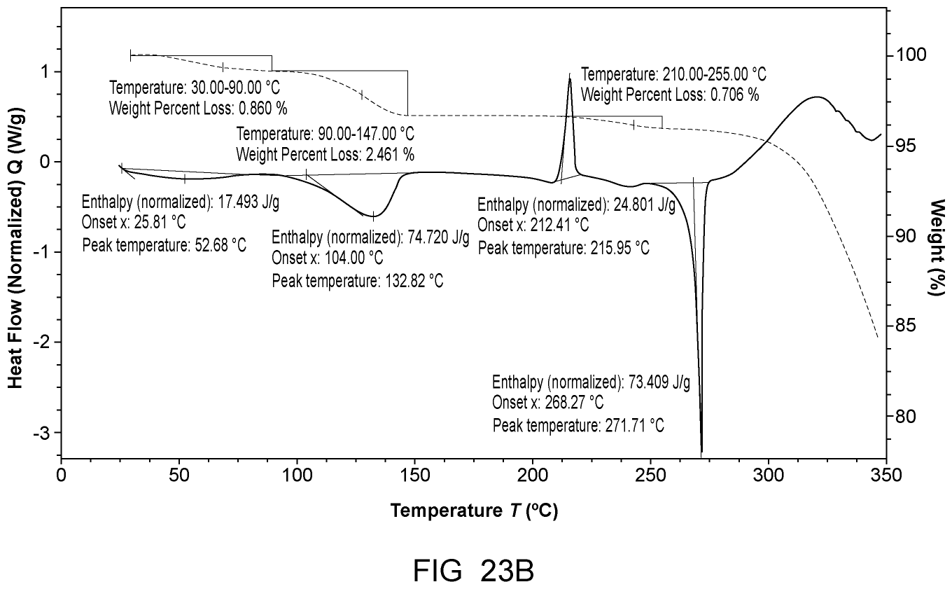

In some embodiments, Compound I Tosylate Form I is further characterized by:
i) one or more peaks at (±0.2°) at 6.6, 13.2, or 16.6 °2θ;
ii) a diffractogram substantially as shown in FIG. 23A;
iii) a differential scanning calorimetry (DSC) curve comprising an endotherm at about 26° C. (onset temperature), an endotherm at about 104° C. (onset temperature), and an endotherm at about 268° C. (onset temperature);
iv) a differential scanning calorimetry (DSC) curve substantially as shown in FIG. 23B;
v) thermogravimetric analysis (TGA) showing about 0.9% weight loss up to about 90° C., about 2.5% weight loss from about 90° C. to about 147° C., and about 0.7% weight loss from about 210° C. to about 255° C.; or
vi) thermogravimetric analysis (TGA) comprising a thermogram substantially as shown in FIG. 23B.

In some embodiments, Compound I Tosylate Form I is further characterized by an X-ray powder diffractogram comprising one or more peaks at (±0.2°) at 6.6, 13.2, or 16.6 °2θ. In some embodiments, Compound I Tosylate Form I is further characterized by an X-ray powder diffractogram comprising one or more peaks at (±0.2°) at 10.7, 12.2, or 19.2 °2θ as determined on a diffractometer using Cu-Kα radiation. In some embodiments, Compound I Tosylate Form I is further characterized by a diffractogram substantially as shown in FIG. 23A.

In some embodiments, Compound I Tosylate Form I is further characterized by a DSC curve comprising an endotherm at about 26° C. (onset temperature), an endotherm at about 104° C. (onset temperature), and an endotherm at about 268° C. (onset temperature). In some embodiments, Compound I Tosylate Form I is further characterized by a DSC curve comprising an endotherm at about 26° C. (onset temperature), an endotherm at about 104° C. (onset temperature), an exotherm at 212° C. (onset temperature), and an endotherm at about 268° C. (onset temperature). In some embodiments, Compound I Tosylate Form I is further characterized by a DSC curve comprising an endotherm at about 53° C. (peak), an endotherm at about 133° C. (peak), an exotherm at about 216° C. (peak), and an endotherm at about 272° C. (peak). In some embodiments, Compound I Tosylate Form I is further characterized by a DSC curve substantially as shown in FIG. 23B.

In some embodiments, Compound I Tosylate Form I is further characterized by a TGA showing about 0.9% weight loss up to about 90° C., about 2.5% weight loss from about 90° C. to about 147° C., and about 0.7% weight loss from about 210° C. to about 255° C. In some embodiments, Compound I Tosylate Form I is further characterized by a TGA comprising a thermogram substantially as shown in FIG. 23B.

In some embodiments, Compound I Tosylate Form I is a hydrate.

### Compound I Mesylate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide mesylate salt Form I (Compound I Mesylate Form I) characterized by an X-ray powder diffractogram comprising peaks (±0.2°) at 11.0, 12.7, and 15.3 °2θ as determined on a diffractometer using Cu-Kα radiation.

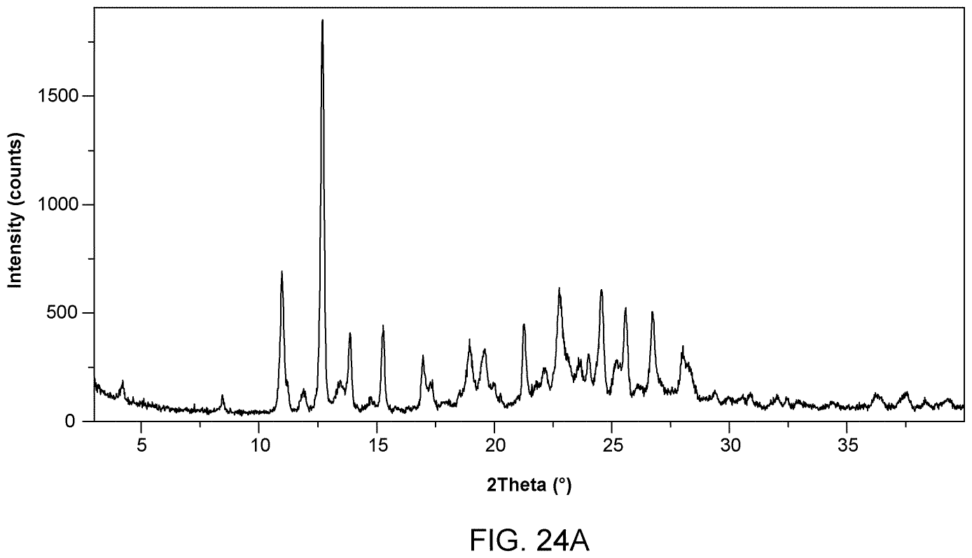

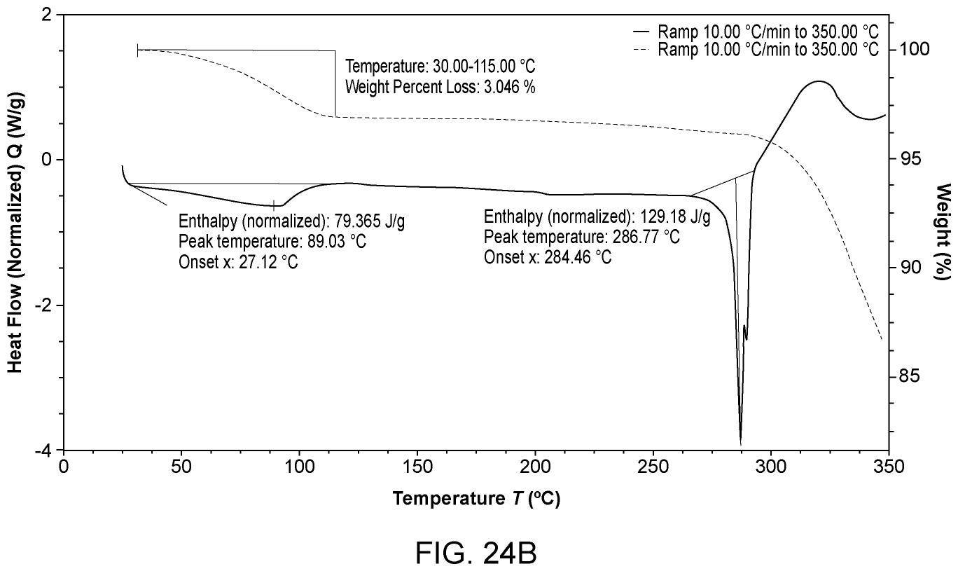

In some embodiments, Compound I Mesylate Form I is further characterized by:
i) one or more peaks at (±0.2°) at 13.9, 18.9, or 21.3 °2θ;
ii) a diffractogram substantially as shown in FIG. 24A;
iii) a differential scanning calorimetry (DSC) curve comprising an endotherm at about 27° C. (onset temperature) and an endotherm at about 284° C. (onset temperature);
iv) a differential scanning calorimetry (DSC) curve substantially as shown in FIG. 24B;
v) thermogravimetric analysis (TGA) showing about 3.0% weight loss up to about 115° C.; or
vi) thermogravimetric analysis (TGA) comprising a thermogram substantially as shown in FIG. 24B.

In some embodiments, Compound I Mesylate Form I is further characterized by an X-ray powder diffractogram comprising one or more peaks at (±0.2°) at 13.9, 18.9, or 21.3 °2θ. In some embodiments, Compound I Mesylate Form I is further characterized by an X-ray powder diffractogram comprising one or more peaks at (±0.2°) at 8.4, 19.6, or 22.7 °2θ as determined on a diffractometer using Cu-Kα radiation. In some embodiments, Compound I Mesylate Form I is further characterized by a diffractogram substantially as shown in FIG. 24A.

In some embodiments, Compound I Mesylate Form I is further characterized by DSC curve comprising an endotherm at about 27° C. (onset temperature) and an endotherm at about 284° C. (onset temperature). In some embodiments, Compound I Mesylate Form I is further characterized by a DSC curve comprising an endotherm at about 89° C. (peak) and an endotherm at about 287° C. (peak). In some embodiments, Compound I Mesylate Form I is further characterized by a DSC curve substantially as shown in FIG. 24B.

In some embodiments, Compound I Mesylate Form I is further characterized by a TGA showing about 3.0% weight loss up to about 115° C. In some embodiments, Compound I Mesylate Form I is further characterized by a TGA comprising a thermogram substantially as shown in FIG. 24B.

In some embodiments, Compound I Mesylate Form I is a hydrate.

### Compound I Phosphate Form III

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form III (Compound I Phosphate Form III) characterized by an X-ray powder diffractogram comprising peaks (±0.2°) at 6.1, 17.1, and 18.1 °2θ as determined on a diffractometer using Cu-Kα radiation.

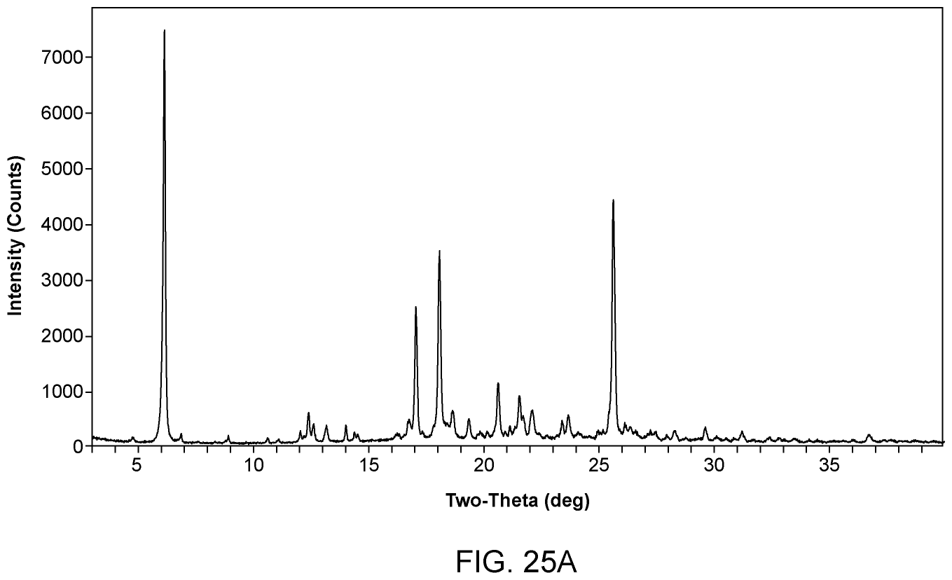

In some embodiments, Compound I Phosphate Form III is further characterized by:
i) one or more peaks at (±0.2°) at 12.4, 20.6, or 25.6 °2θ; or
ii) a diffractogram substantially as shown in FIG. 25A.

In some embodiments, Compound I Phosphate Form III is further characterized by an X-ray powder diffractogram comprising one or more peaks at (±0.2°) at 12.4, 20.6, or 25.6 °2θ. In some embodiments, Compound I Phosphate Form III is further characterized by an X-ray powder diffractogram comprising one or more peaks at (±0.2°) at 13.2, 14.0, or 18.6 °2θ as determined on a diffractometer using Cu-Kα radiation. In some embodiments, Compound I Phosphate Form III is further characterized by a diffractogram substantially as shown in FIG. 25A.

### Compound I Phosphate Form IV

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form IV (Compound I Phosphate Form IV) characterized by an X-ray powder diffractogram comprising peaks (±0.2°) at 5.1, 8.3, and 13.0 °2θ as determined on a diffractometer using Cu-Kα radiation.

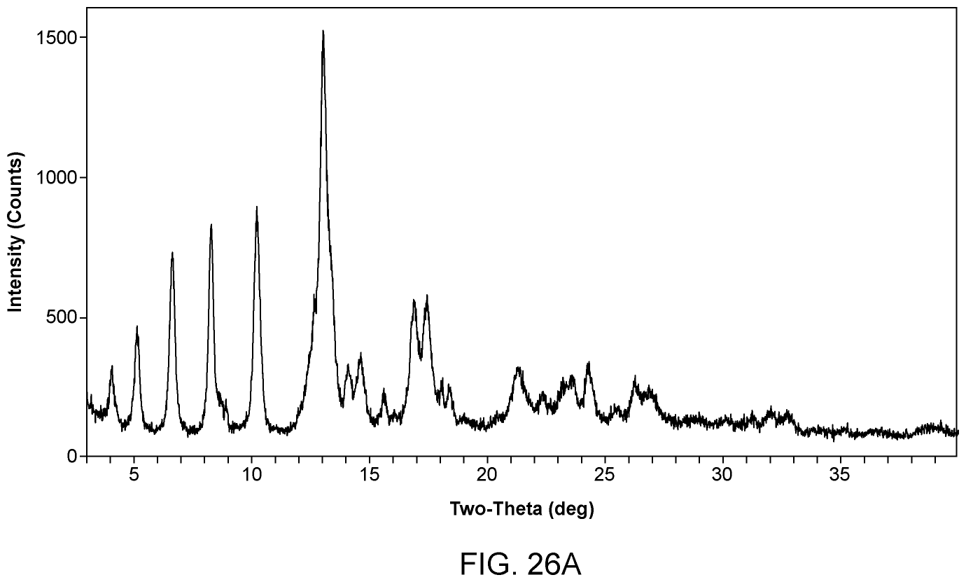

In some embodiments, Compound I Phosphate Form IV is further characterized by:
i) one or more peaks at (±0.2°) at 6.6, 10.2, or 14.6 °2θ; or
ii) a diffractogram substantially as shown in FIG. 26A.

In some embodiments, Compound I Phosphate Form IV is further characterized by an X-ray powder diffractogram comprising one or more peaks at (±0.2°) at 6.6, 10.2, or 14.6 °2θ. In some embodiments, Compound I Phosphate Form IV is further characterized by an X-ray powder diffractogram comprising one or more peaks at (±0.2°) at 14.1, 16.9, or 17.4 °2θ as determined on a diffractometer using Cu-Kα radiation. In some embodiments, Compound I Phosphate Form IV is further characterized by a diffractogram substantially as shown in FIG. 26A.

### Compound I Phosphate Form V

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form V (Compound I Phosphate Form V) characterized by an X-ray powder diffractogram comprising peaks (±0.2°) at 4.8, 11.8, and 18.5 °2θ as determined on a diffractometer using Cu-Kα radiation.

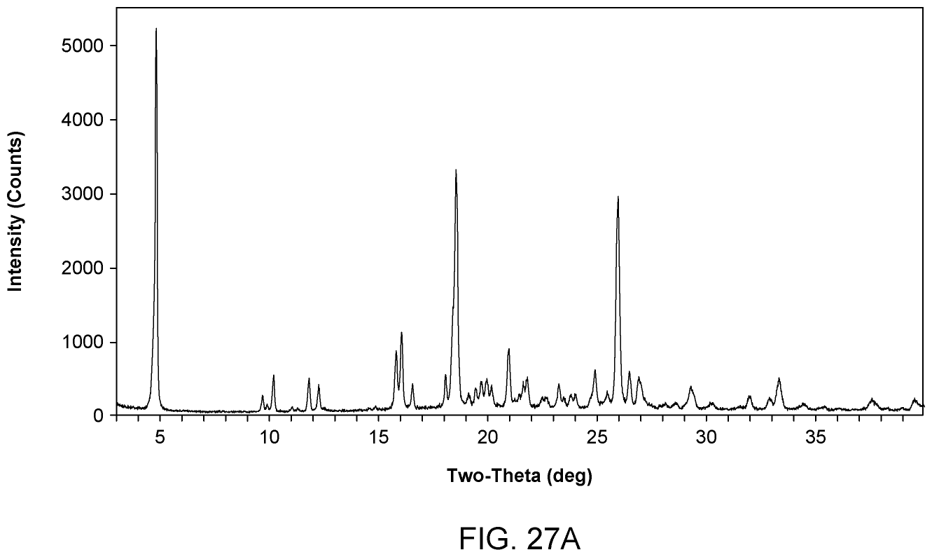

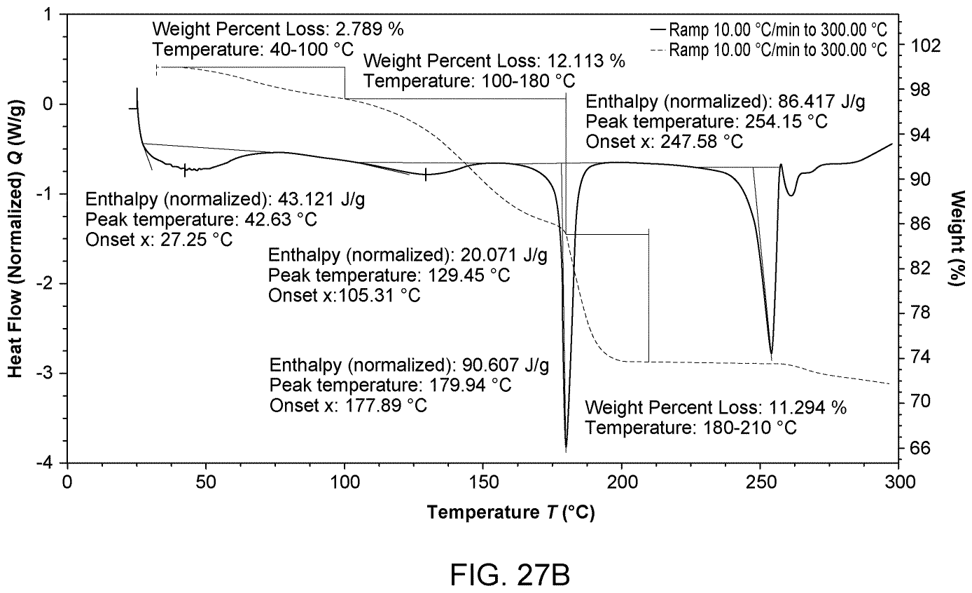

In some embodiments, Compound I Phosphate Form V is further characterized by:
i) one or more peaks at (±0.2°) at 10.2, 12.3, or 16.1 °2θ;
ii) a diffractogram substantially as shown in FIG. 27A;
iii) a differential scanning calorimetry (DSC) curve comprising an endotherm at about 27° C. (onset temperature), an endotherm at about 105° C. (onset temperature), an endotherm at about 178° C. (onset temperature), and an endotherm at about 248° C. (onset temperature);
iv) a differential scanning calorimetry (DSC) curve substantially as shown in FIG. 27B;
v) thermogravimetric analysis (TGA) showing about 2.8% weight loss up to about 100° C., about 12.1% weight loss from about 100° C. to about 180° C., and about 11.3% weight loss from about 180° C. to about 210° C.; or
vi) thermogravimetric analysis (TGA) comprising a thermogram substantially as shown in FIG. 27B.

In some embodiments, Compound I Phosphate Form V is further characterized by an X-ray powder diffractogram comprising one or more peaks at (±0.2°) at 10.2, 12.3, or 16.1 °2θ. In some embodiments, Compound I Phosphate Form V is further characterized by an X-ray powder diffractogram comprising one or more peaks at (±0.2°) at 15.8, 16.6, or 21.0 °2θ as determined on a diffractometer using Cu-Kα radiation. In some embodiments, Compound I Phosphate Form V is further characterized by a diffractogram substantially as shown in FIG. 27A.

In some embodiments, Compound I Phosphate Form V is further characterized by a DSC curve comprising an endotherm at about 27° C. (onset temperature), an endotherm at about 105° C. (onset temperature), an endotherm at about 178° C. (onset temperature), and an endotherm at about 248° C. (onset temperature). In some embodiments, Compound I Phosphate Form V is further characterized by a DSC curve comprising an endotherm at about 43° C. (peak), an endotherm at about 129° C. (peak), an endotherm at about 180° C. (peak), and an endotherm at about 254° C. (peak). In some embodiments, Compound I Phosphate Form V is further characterized by a DSC curve substantially as shown in FIG. 27B.

In some embodiments, Compound I Phosphate Form V is further characterized by TGA showing about 2.8% weight loss up to about 100° C., about 12.1% weight loss from about 100° C. to about 180° C., and about 11.3% weight loss from about 180° C. to about 210° C. In some embodiments, Compound I Phosphate Form V is further characterized by TGA comprising a thermogram substantially as shown in FIG. 27B.

In some embodiments, Compound I Phosphate Form V is a solvate. In some embodiments, Compound I Phosphate Form V is a N-methylpyrrolidone (NMP) solvate.

### Compound I Phosphate Form VI

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form VI (Compound I Phosphate Form VI) characterized by an X-ray powder diffractogram comprising peaks (±0.2°) at 5.6, 11.3, and 18.2 °2θ as determined on a diffractometer using Cu-Kα radiation.

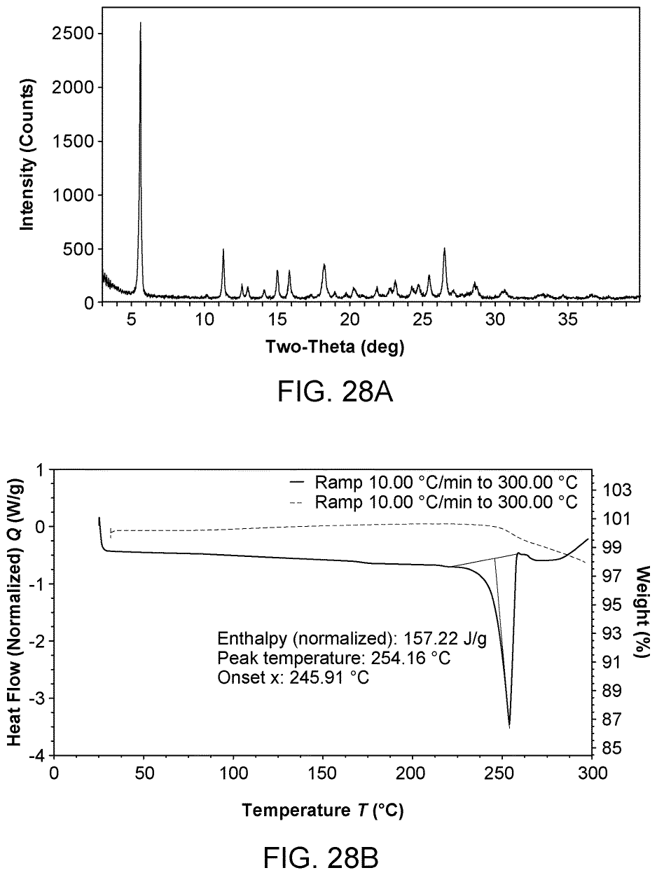

In some embodiments, Compound I Phosphate Form VI is further characterized by:
i) one or more peaks at (±0.2°) at 12.6, 15.0, or 15.8 °2θ;
ii) a diffractogram substantially as shown in FIG. 28A;
iii) a differential scanning calorimetry (DSC) curve comprising an endotherm at about 246° C. (onset temperature);
iv) a differential scanning calorimetry (DSC) curve substantially as shown in FIG. 28B;
v) thermogravimetric analysis (TGA) showing negligible weight loss; or
vi) thermogravimetric analysis (TGA) comprising a thermogram substantially as shown in FIG. 28B.

In some embodiments, Compound I Phosphate Form VI is further characterized by an X-ray powder diffractogram comprising one or more peaks at (±0.2°) at 12.6, 15.0, or 15.8 °2θ. In some embodiments, Compound I Phosphate Form VI is further characterized by an X-ray powder diffractogram comprising one or more peaks at (±0.2°) at 13.0, 14.1, or 21.9 °2θ as determined on a diffractometer using Cu-Kα radiation. In some embodiments, Compound I Phosphate Form VI is further characterized by a diffractogram substantially as shown in FIG. 28A.

In some embodiments, Compound I Phosphate Form VI is further characterized by a DSC curve comprising an endotherm at about 246° C. (onset temperature). In some embodiments, Compound I Phosphate Form VI is further characterized by a DSC curve comprising an endotherm at about 254° C. (peak). In some embodiments, Compound I Phosphate Form VI is further characterized by a DSC curve substantially as shown in FIG. 28B.

In some embodiments, Compound I Phosphate Form VI is further characterized by TGA showing negligible weight loss. In some embodiments, Compound I Phosphate Form VI is further characterized by TGA comprising a thermogram substantially as shown in FIG. 28B.

In some embodiments, Compound I Phosphate Form VI is an anhydrate.

### Compound I Phosphate Form VII

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide phosphate salt Form VII (Compound I Phosphate Form VII) characterized by an X-ray powder diffractogram comprising peaks (±0.2°) at 9.3, 11.8, and 14.6 °2θ as determined on a diffractometer using Cu-Kα radiation.

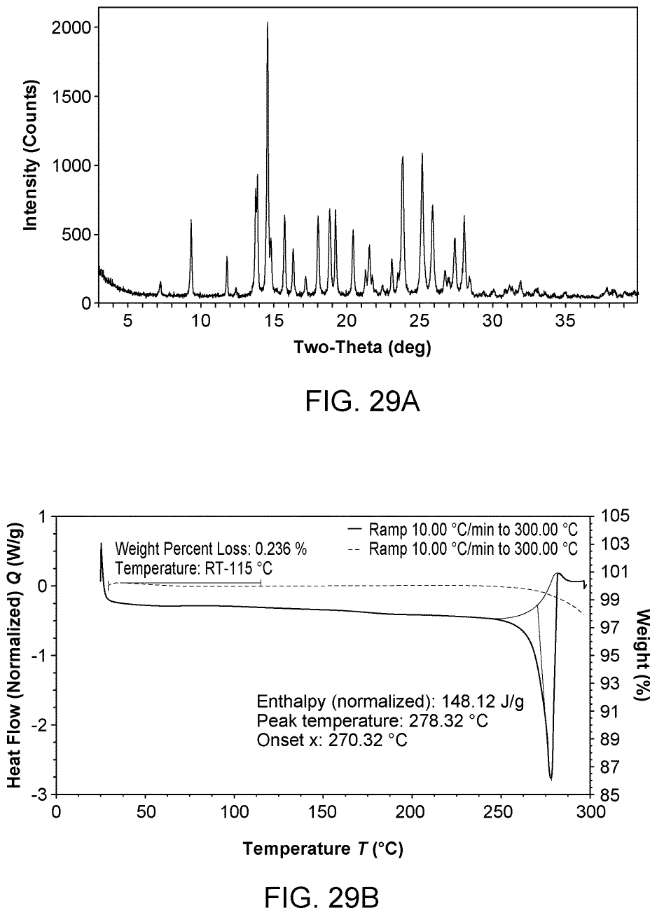

In some embodiments, Compound I Phosphate Form VII is further characterized by:
i) one or more peaks at (±0.2°) at 13.9, 15.7, or 18.0 °2θ;
ii) a diffractogram substantially as shown in FIG. 29A;
iii) a differential scanning calorimetry (DSC) curve comprising an endotherm at about 270° C. (onset temperature);
iv) a differential scanning calorimetry (DSC) curve substantially as shown in FIG. 29B;
v) thermogravimetric analysis (TGA) showing about 0.2% weight loss up to about 115° C.; or
vi) thermogravimetric analysis (TGA) comprising a thermogram substantially as shown in FIG. 29B.

In some embodiments, Compound I Phosphate Form VII is further characterized by an X-ray powder diffractogram comprising one or more peaks at (±0.2°) at 13.9, 15.7, or 18.0 °2θ. In some embodiments, Compound I Phosphate Form VII is further characterized by an X-ray powder diffractogram comprising one or more peaks at (±0.2°) at 7.2, 16.3, or 19.2 °2θ as determined on a diffractometer using Cu-Kα radiation. In some embodiments, Compound I Phosphate Form VII is further characterized by a diffractogram substantially as shown in FIG. 29A.

In some embodiments, Compound I Phosphate Form VII is further characterized by a DSC curve comprising an endotherm at about 270° C. (onset temperature). In some embodiments, Compound I Phosphate Form VII is further characterized by a DSC curve comprising an endotherm at about 278° C. (peak). In some embodiments, Compound I Phosphate Form VII is further characterized by a DSC curve substantially as shown in FIG. 29B.

In some embodiments, Compound I Phosphate Form VII is further characterized by TGA showing about 0.2% weight loss up to about 115° C. In some embodiments, Compound I Phosphate Form VII is further characterized by TGA comprising a thermogram substantially as shown in FIG. 29B.

In some embodiments, Compound I Phosphate Form VII is an anhydrate.

### Compound I Malonate Acetonitrile Solvate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide malonate salt Form I (Compound I Malonate Acetonitrile Solvate Form I) characterized by an X-ray powder diffractogram comprising peaks (±0.2°) at 5.4, 9.8, and 15.4 °2θ as determined on a diffractometer using Cu-Kα radiation.

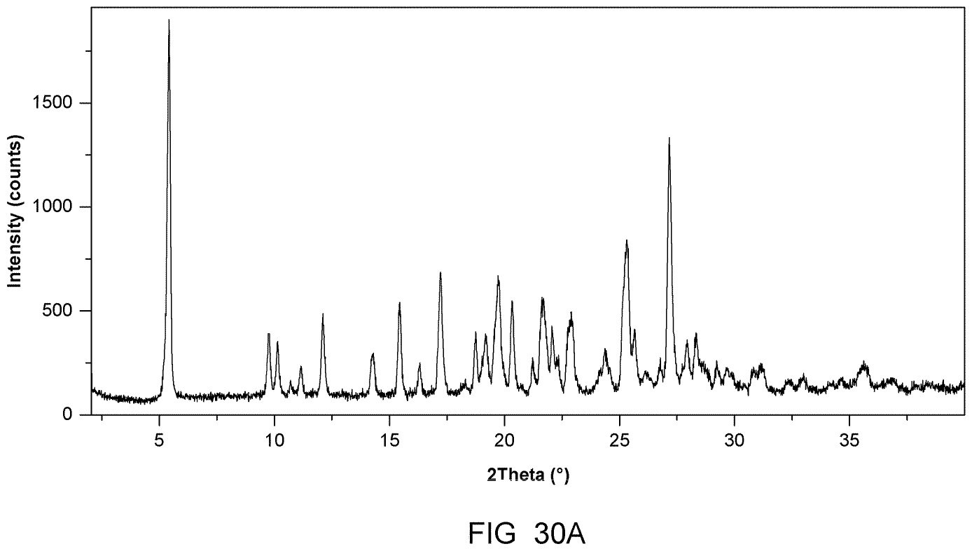

In some embodiments, Compound I Malonate Acetonitrile Solvate Form I is further characterized by:
i) one or more peaks at (±0.2°) at 10.1, 12.1, or 17.2 °2θ; or
ii) a diffractogram substantially as shown in FIG. 30A.

In some embodiments, Compound I Malonate Acetonitrile Solvate Form I is further characterized by an X-ray powder diffractogram comprising one or more peaks at (±0.2°) at 10.1, 12.1, or 17.2 °2θ. In some embodiments, Compound I Malonate Acetonitrile Solvate Form I is further characterized by an X-ray powder diffractogram comprising one or more peaks at (±0.2°) at 14.4, 16.3, or 19.8 °2θ as determined on a diffractometer using Cu-Kα radiation. In some embodiments, Compound I Malonate Acetonitrile Solvate Form I is further characterized by a diffractogram substantially as shown in FIG. 30A.

### Compound I Malonate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide malonate salt Form I (Compound I Malonate Form I) characterized an X-ray powder diffractogram comprising peaks (±0.2°) at 5.7, 12.0, and 15.2 °2θ as determined on a diffractometer using Cu-Kα radiation.

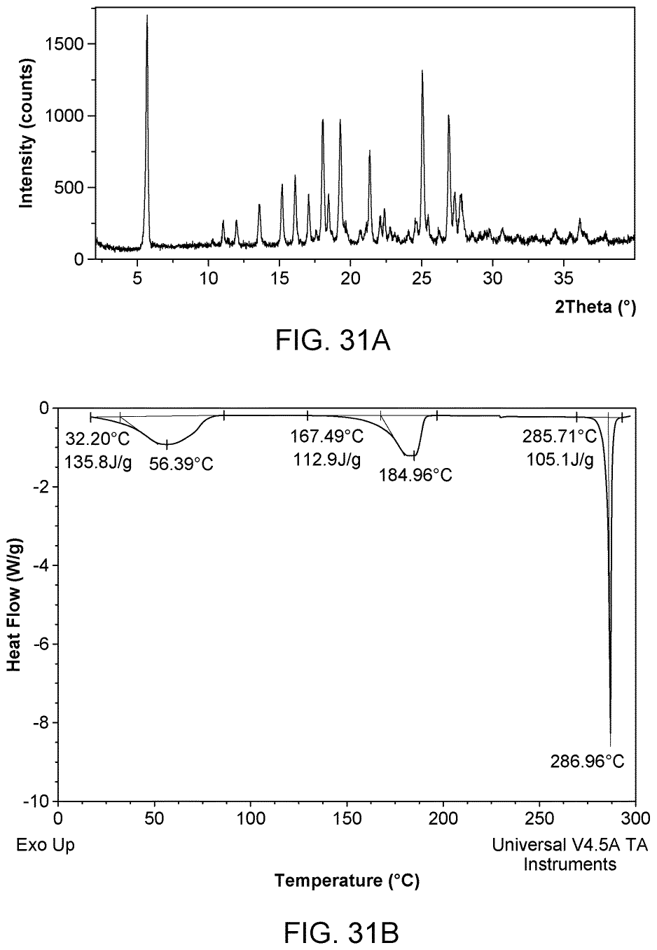

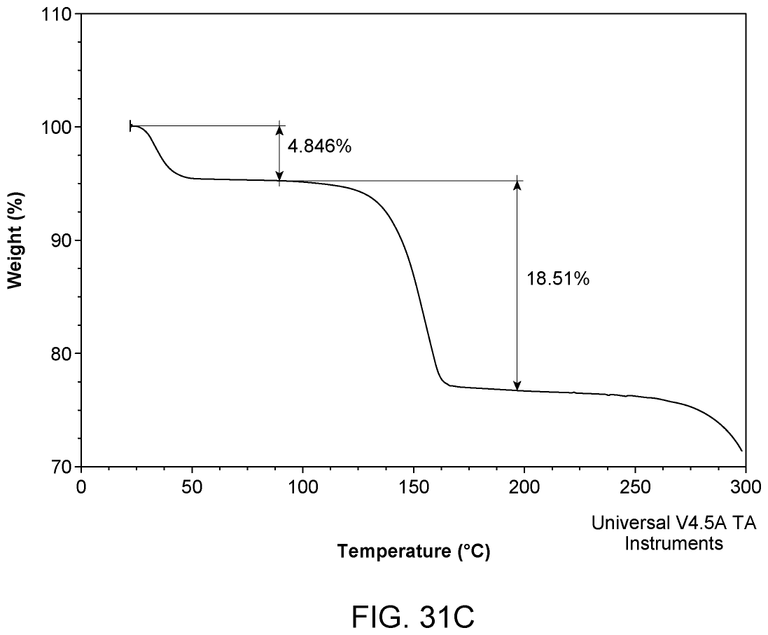

In some embodiments, Compound I Malonate Form I is further characterized by:
i) one or more peaks at (±0.2°) at 11.1, 16.1, or 18.0 °2θ;
ii) a diffractogram substantially as shown in FIG. 31A;
iii) a differential scanning calorimetry (DSC) curve comprising an endotherm at about 32° C. (onset temperature), an endotherm at about 168° C. (onset temperature), and an endotherm at about 286° C. (onset temperature);
iv) a differential scanning calorimetry (DSC) curve substantially as shown in FIG. 31B;
v) thermogravimetric analysis (TGA) showing a weight loss of about 4.8 wt % up to about 90° C. and a weight loss of about 18.5% from 90° C. up to about 200° C.; or
vi) thermogravimetric analysis (TGA) comprising a thermogram substantially as shown in FIG. 31C.

In some embodiments, Compound I Malonate Form I is further characterized by an X-ray powder diffractogram comprising one or more peaks at (±0.2°) at 11.1, 16.1, or 18.0 °2θ. In some embodiments, Compound I Malonate Form I is further characterized by an X-ray powder diffractogram comprising one or more peaks at (±0.2°) at 13.6, 17.0, or 19.3 °2θ as determined on a diffractometer using Cu-Kα radiation. In some embodiments, Compound I Malonate Form I is further characterized by a diffractogram substantially as shown in FIG. 31A.

In some embodiments, Compound I Malonate Form I is further characterized by a DSC curve comprising an endotherm at about 32° C. (onset temperature), an endotherm at about 168° C. (onset temperature), and an endotherm at about 286° C. (onset temperature). In some embodiments, Compound I Malonate Form I is further characterized by a DSC curve comprising an endotherm at about 56° C. (peak), an endotherm at about 185° C. (peak), and an endotherm at about 287° C. (peak). In some embodiments, Compound I Malonate Form I is further characterized by a DSC curve substantially as shown in FIG. 31B.

In some embodiments, Compound I Malonate Form I is further characterized by TGA showing a weight loss of about 4.8 wt % up to about 90° C. and a weight loss of about 18.5% from 90° C. up to about 200° C. In some embodiments, Compound I Malonate Form I is further characterized by TGA comprising a thermogram substantially as shown in FIG. 31C.

### Compound I Citrate Methanol Solvate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide citrate salt Form I (Compound I Citrate Methanol Solvate Form I) characterized by an X-ray powder diffractogram comprising peaks (±0.2°) at 4.8, 9.8, and 14.7 °2θ as determined on a diffractometer using Cu-Kα radiation.

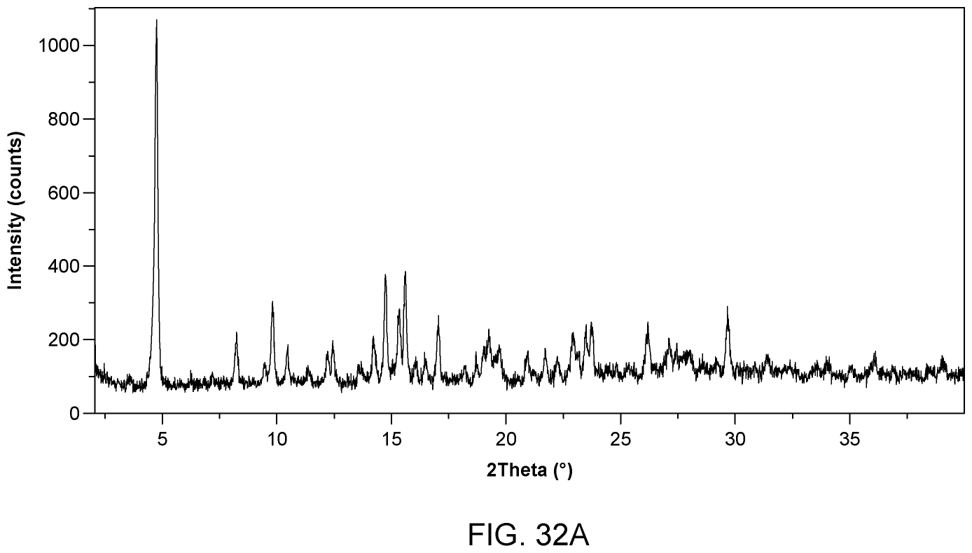

In some embodiments, Compound I Citrate Methanol Solvate Form I is further characterized by:
i) one or more peaks at (±0.2°) at 8.2, 10.5, or 15.6 °2θ; or
ii) a diffractogram substantially as shown in FIG. 32A.

In some embodiments, Compound I Citrate Methanol Solvate Form I is further characterized by an X-ray powder diffractogram comprising one or more peaks at (±0.2°) at 8.2, 10.5, or 15.6 °2θ. In some embodiments, Compound I Citrate Methanol Solvate Form I is further characterized by an X-ray powder diffractogram comprising one or more peaks at (±0.2°) at 12.4, 15.3, or 17.0 °2θ as determined on a diffractometer using Cu-Kα radiation. In some embodiments, Compound I Citrate Methanol Solvate Form I is further characterized by a diffractogram substantially as shown in FIG. 32A.

### Compound I Citrate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide citrate salt Form I (Compound I Citrate Form I) characterized by an X-ray powder diffractogram comprising peaks (±0.2°) at 5.3, 10.5, and 16.6 °2θ as determined on a diffractometer using Cu-Kα radiation.

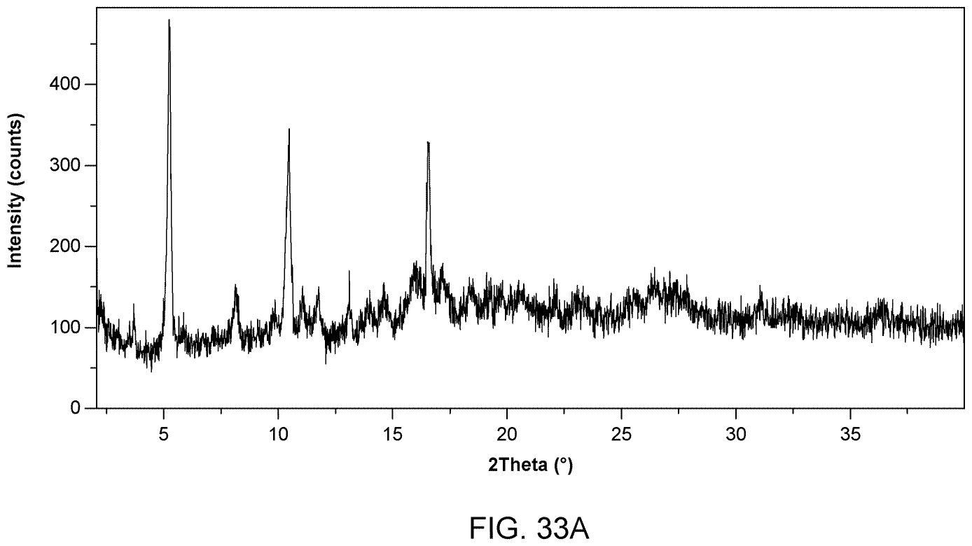

In some embodiments, Compound I Citrate Form I is further characterized by:
i) one or more peaks at (±0.2°) at 8.2, 11.1, or 16.0 °2θ; or
ii) a diffractogram substantially as shown in FIG. 33A.

In some embodiments, Compound I Citrate Form I is further characterized by an X-ray powder diffractogram comprising one or more peaks at (±0.2°) at 8.2, 11.1, or 16.0 °2θ. In some embodiments, Compound I Citrate Form I is further characterized by an X-ray powder diffractogram comprising one or more peaks at (±0.2°) at 3.7, 11.7, or 13.1 °2θ as determined on a diffractometer using Cu-Kα radiation. In some embodiments, Compound I Citrate Form I is further characterized by a diffractogram substantially as shown in FIG. 33A.

### Compound I Citrate Form II

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide citrate salt Form II (Compound I Citrate Form II) characterized by an X-ray powder diffractogram comprising peaks (±0.2°) at 5.2, 10.3, and 14.2 °2θ as determined on a diffractometer using Cu-Kα radiation.

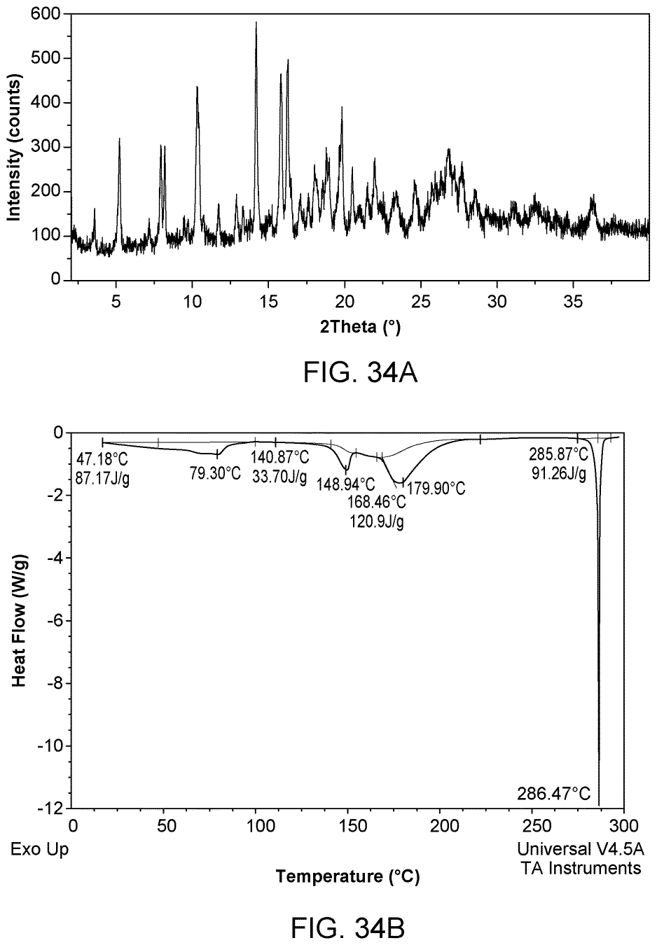

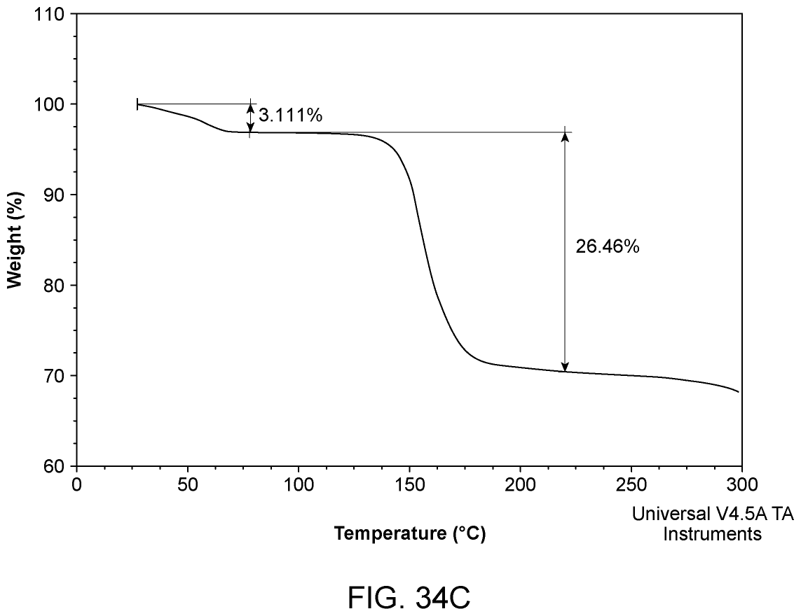

In some embodiments, Compound I Citrate Form II is further characterized by:
i) one or more peaks at (±0.2°) at 7.9, 15.8, or 16.3 °2θ;
ii) a diffractogram substantially as shown in FIG. 34A;
iii) a differential scanning calorimetry (DSC) curve comprising an endotherm at about 47° C. (onset temperature), an endotherm at about 141° C. (onset temperature), an endotherm at about 169° C. (onset temperature), and an endotherm at about 286° C. (onset temperature);
iv) a differential scanning calorimetry (DSC) curve substantially as shown in FIG. 34B;
v) thermogravimetric analysis (TGA) showing a weight loss of about 3.1 wt % up to about 75° C. and a weight loss of about 26.5% from 75° C. up to about 220° C.; or
vi) thermogravimetric analysis (TGA) comprising a thermogram substantially as shown in FIG. 34C.

In some embodiments, Compound I Citrate Form II is further characterized by an X-ray powder diffractogram comprising one or more peaks at (±0.2°) at 7.9, 15.8, or 16.3 °2θ. In some embodiments, Compound I Citrate Form II is further characterized by an X-ray powder diffractogram comprising one or more peaks at (±0.2°) at 3.6, 8.2, or 19.8 °2θ as determined on a diffractometer using Cu-Kα radiation. In some embodiments, Compound I Citrate Form II is further characterized by a diffractogram substantially as shown in FIG. 34A.

In some embodiments, Compound I Citrate Form II is further characterized by a DSC curve comprising an endotherm at about 47° C. (onset temperature), an endotherm at about 141° C. (onset temperature), an endotherm at about 169° C. (onset temperature), and an endotherm at about 286° C. (onset temperature). In some embodiments, Compound I Citrate Form II is further characterized by a DSC curve comprising an endotherm at about 79° C. (peak), an endotherm at about 149° C. (peak), an endotherm at about 180° C. (peak), and an endotherm at about 286° C. (peak). In some embodiments, Compound I Citrate Form II is further characterized by a DSC curve substantially as shown in FIG. 34B.

In some embodiments, Compound I Citrate Form II is further characterized by TGA showing a weight loss of about 3.1 wt % up to about 75° C. and a weight loss of about 26.5% from 75° C. up to about 220° C. In some embodiments, Compound I Citrate Form II is further characterized by TGA comprising a thermogram substantially as shown in FIG. 34C.

### Compound I Oxalate Acetonitrile Solvate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide oxalate salt Form I (Compound I Oxalate Acetonitrile Solvate Form I) characterized by an X-ray powder diffractogram comprising peaks (±0.2°) at 5.1, 15.4, and 16.6 °2θ as determined on a diffractometer using Cu-Kα radiation.

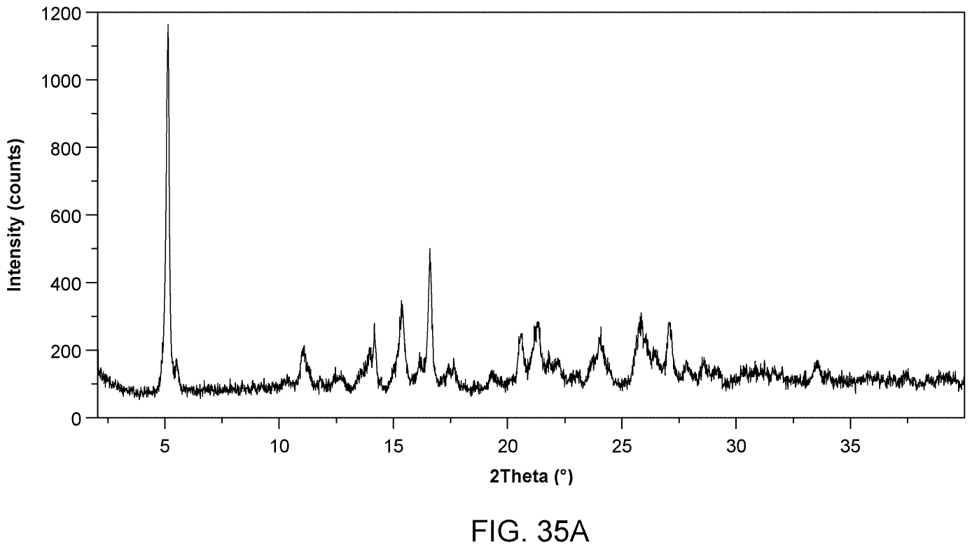

In some embodiments, Compound I Oxalate Acetonitrile Solvate Form I is further characterized by:
i) one or more peaks at (±0.2°) at 11.0, 14.2, or 21.3 °2θ; or
ii) a diffractogram substantially as shown in FIG. 35A.

In some embodiments, Compound I Oxalate Acetonitrile Solvate Form I is further characterized by an X-ray powder diffractogram comprising one or more peaks at (±0.2°) at 11.0, 14.2, or 21.3 °2θ. In some embodiments, Compound I Oxalate Acetonitrile Solvate Form I is further characterized by an X-ray powder diffractogram comprising one or more peaks at (±0.2°) at 20.6, 24.0, or 27.1 °2θ as determined on a diffractometer using Cu-Kα radiation. In some embodiments, Compound I Oxalate Acetonitrile Solvate Form I is further characterized by a diffractogram substantially as shown in FIG. 35A.

### Compound I Oxalate Form I

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide oxalate salt Form I (Compound I Oxalate Form I) characterized by an X-ray powder diffractogram comprising peaks (±0.2°) at 5.6, 12.9, and 20.6 °2θ as determined on a diffractometer using Cu-Kα radiation.

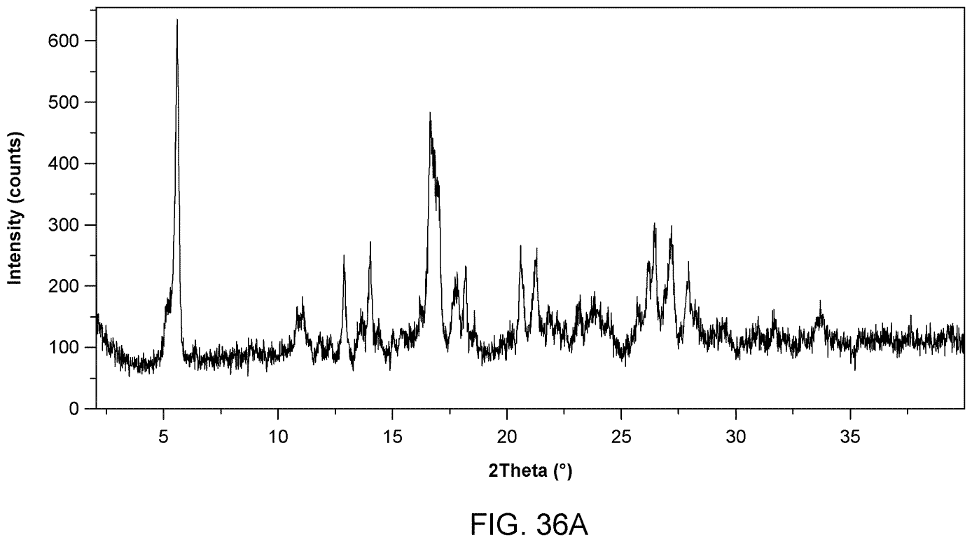

In some embodiments, Compound I Oxalate Form I is further characterized by:
i) one or more peaks at (±0.2°) at 11.0, 14.0, or 21.3 °2θ; or
ii) a diffractogram substantially as shown in FIG. 36A.

In some embodiments, Compound I Oxalate Form I is further characterized by an X-ray powder diffractogram comprising one or more peaks at (±0.2°) at 11.0, 14.0 or 21.3 °2θ. In some embodiments, Compound I Oxalate Form I is further characterized by an X-ray powder diffractogram comprising one or more peaks at (±0.2°) at 16.6, 18.2, or 27.2 °2θ as determined on a diffractometer using Cu-Kα radiation. In some embodiments, Compound I Oxalate Form I is further characterized by a diffractogram substantially as shown in FIG. 36A.

### Compound I Fumarate Form II

In some embodiments, provided is crystalline 6-fluoro-5-[4-({6-fluoro-4-oxo-2H,3H,5H-furo[3,2-c]quinolin-7-yl} methyl) piperazin-1-yl]-N-methylpyridine-2-carboxamide fumarate salt Form II (Compound I Fumarate Form II) characterized by an X-ray powder diffractogram comprising peaks (±0.2°) at 4.2, 8.5, and 12.7 °2θ as determined on a diffractometer using Cu-Kα radiation.

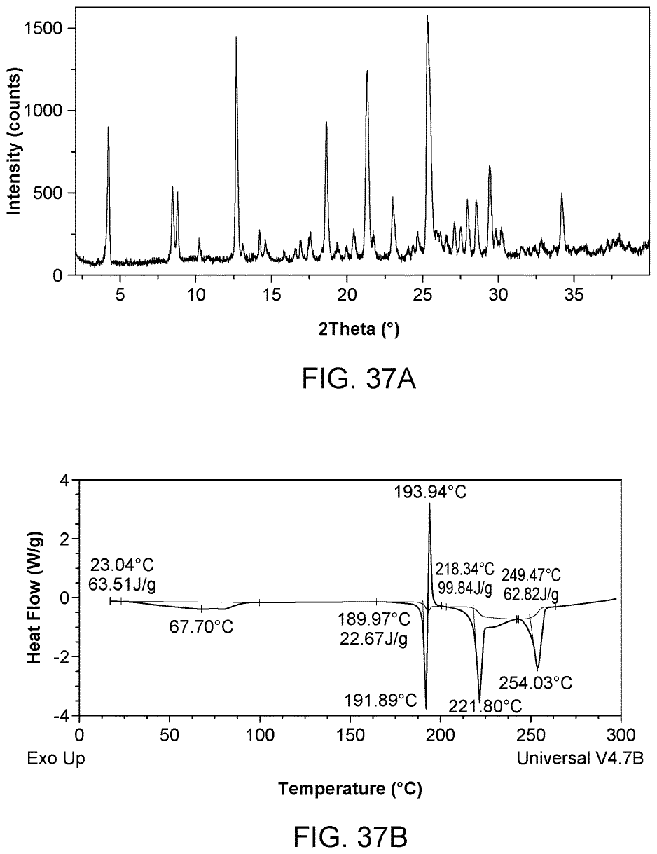

In some embodiments, Compound I Fumarate Form II is further characterized by:
i) one or more peaks at (±0.2°) at 8.8, 14.2, or 18.6 °2θ;
ii) a diffractogram substantially as shown in FIG. 37A;
iii) a differential scanning calorimetry (DSC) curve comprising an endotherm at about 23° C. (onset temperature), an endotherm at about 190° C. (onset temperature), an endotherm at about 218° C. (onset temperature), and an endotherm at about 250° C. (onset temperature); or
iv) a differential scanning calorimetry (DSC) curve substantially as shown in FIG. 37B.

In some embodiments, Compound I Fumarate Form II is further characterized by an X-ray powder diffractogram comprising one or more peaks at (±0.2°) at 8.8, 14.2, or 18.6 °2θ. In some embodiments, Compound I Fumarate Form II is further characterized by an X-ray powder diffractogram comprising one or more peaks at (±0.2°) at 10.2, 17.6, or 21.3 °2θ as determined on a diffractometer using Cu-Kα radiation. In some embodiments, Compound I Fumarate Form II is further characterized by a diffractogram substantially as shown in FIG. 37A.

In some embodiments, Compound I Fumarate Form II is further characterized by a DSC curve comprising an endotherm at about 23° C. (onset temperature), an endotherm at about 190° C. (onset temperature), an endotherm at about 218° C. (onset temperature), and an endotherm at about 250° C. (onset temperature). In some embodiments, Compound I Fumarate Form II is further characterized by a DSC curve comprising an endotherm at about 23° C. (onset temperature), an endotherm at about 190° C. (onset temperature), an exotherm at about 192° C. (onset temperature), an endotherm at about 218° C. (onset temperature), and an endotherm at about 250° C. (onset temperature). In some embodiments, Compound I Fumarate Form II is further characterized by a DSC curve comprising an endotherm at about 68° C. (peak), an endotherm at about 192° C. (peak), an endotherm at about 222° C. (peak), and an endotherm at about 254° C. (peak). In some embodiments, Compound I Fumarate Form II is further characterized by a DSC curve comprising an endotherm at about 68° C. (peak), an endotherm at about 192° C. (peak), an exotherm at about 194° C. (peak), an endotherm at about 222° C. (peak), and an endotherm at about 254° C. (peak). In some embodiments, Compound I Fumarate Form II is further characterized by a DSC curve substantially as shown in FIG. 37B.

### Method of Treatment

Disclosed herein are methods of treatment of a disease in which inhibition of PARP is beneficial, the method comprising administering a compound or crystalline form disclosed herein. Also disclosed herein are methods of treatment of a disease in which inhibition of PARP1 is beneficial, the method comprising administering a compound or crystalline form disclosed herein. In some embodiments, the disease is cancer. In some embodiments, the cancer is breast cancer, ovarian cancer, pancreatic cancer, prostate cancer, a hematological cancer, a gastrointestinal cancer such as gastric cancer and colorectal cancer, or lung cancer. In some embodiments, the cancer is breast cancer, ovarian cancer, pancreatic cancer, or prostate cancer. In some embodiment, the cancer is leukemia, colon cancer, glioblastoma, lymphoma, melanoma, or cervical cancer.

In some embodiments, the cancer comprises a BRCA1 and/or a BRCA2 mutation.

In some embodiments, the cancer comprising a BRCA1 and/or a BRCA2 mutation is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

In some embodiments, the cancer is a cancer deficient in Flomologous Recombination (FIR) dependent DNA DSB repair activity. The FIR dependent DNA DSB repair pathway repairs double-strand breaks (DSBs) in DNA via homologous mechanisms to reform a continuous DNA helix. The components of the FIR dependent DNA DSB repair pathway include, but are not limited to, ATM (NM_000051), RAD51 (NM_002875), RAD51 L1 (NM_002877), RAD51 C (NM_002876), RAD51 L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE1 1 A (NM_005590) and NBS1 (NM_002485). Other proteins involved in the FIR dependent DNA DSB repair pathway include regulatory factors such as EMSY. In some embodiments, the cancer which is deficient in FIR dependent DNA DSB repair comprises one or more cancer cells which have a reduced or abrogated ability to repair DNA DSBs through that pathway, relative to normal cells i.e. the activity of the FIR dependent DNA DSB repair pathway may be reduced or abolished in the one or more cancer cells.

In some embodiments, the activity of one or more components of the FIR dependent DNA DSB repair pathway is abolished in the one or more cancer cells of an individual having a cancer which is deficient in FIR dependent DNA DSB repair.

In some embodiments, the cancer cells have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example, by means of mutation or polymorphism in the encoding nucleic acid, or by means of amplification, mutation or polymorphism in a gene encoding a regulatory factor, for example, the EMSY gene which encodes a BRCA2 regulatory factor. BRCA1 and BRCA2 are known tumor suppressors whose wild-type alleles are frequently lost in tumors of heterozygous carriers. Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast cancer, ovarian cancer, pancreatic cancer, prostate cancer, a hematological cancer, gastrointestinal cancer, and lung cancer.

To minimize the risks of off-target effects, it is desirable for drug molecules to possess selectivity for a specific target.

Avoiding inhibition of PARP family isoforms beyond PARP1 may be important in minimizing toxicities that may arise from inhibition of non-PARP1 isoforms. The pharmacology of inhibiting PARP isoforms beyond PARP1 may drive toxicities that reduce the therapeutic index for agents that possess lower selectivity's for PARP1 against PARP isoforms, PARP3, like PARP1, plays a role in DNA damage but has also been found to be a key player in the integrity of the mitotic spindle and in telomerase integrity (Boehler, C., Gauthier, L R., Mortusewicz O. et al. Poly(ADP-ribose) polymerase 3 (PARP3), a newcomer in cellular response to DNA damage and mitotic progression. PNAS, Jan. 26, 2011, 108 (7) 2783-2788). PARP5A also known as Tankyrase 1, plays key roles in Wnt signaling and telomere length (Kulak, O., Chen, H., Holohan B. et al. Disruption of Wnt/β-Catenin Signaling and Telomeric Shortening Are Inextricable Consequences of Tankyrase Inhibition in Human Cells. Mol Cell Biol. 2015 July; 35(14), 2425-2435). PARP6 is an essential microtubule-regulatory gene in mice, germline mutations in PARP6 that abrogate the catalytic activity has negative effects on neuronal function in humans (Vermehren-Schmaedick, A., Huang J. Y., Levinson, M. et al. Characterization of PARP6 Function in Knockout Mice and Patients with Developmental Delay. Cells, 2021 June; 10(6), 1289). PARP7 catalytic inhibition causes hyper stimulatory effects on type one interferon producing an autoimmune phenotype (Gozgit, J. M., Vasbinder, M. M., Abo, R. P. et al. PARP7 negatively regulates the type I interferon response in cancer cells and its inhibition triggers antitumor immunity. Volume 39, Issue 9, 13 Sep. 2021, Pages 1214-1226). While the exact function of PARP8 has not been established, its knockout has been shown to induce mitotic and nuclear morphology defects and a decrease in cellular viability (Vyas, S., Chesarone-Cataldo, M., Todorova, T., et al. A Systematic Analysis of the PARP Protein Family Identifies New Functions Critical for Cell Physiology. Nat. Commun, 2013, 4 (1), 2240). PARP10 has been described as a MYC interacting protein with tumor suppressor activities (Yu, M. Schreek, S., Cerni, C. et al. PARP-10, a novel Myc-interacting protein with poly(ADP-ribose) polymerase activity, inhibits transformation. Oncogene, 2005 volume 24, pages 1982-1993).

### Dosing

In certain embodiments, the compositions containing Compound I, or a pharmaceutically acceptable salt or solvate thereof, are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion, the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound or crystalline form described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds or crystalline forms described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound or crystalline form described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, Compound I, or a pharmaceutically acceptable salt or solvate thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound or crystalline form as described herein is provided in the form of a rapid release formulation, in the form of an extended release formulation, or in the form of an intermediate release formulation. In yet other embodiments, the compound or crystalline form described herein is administered topically.

### Compositions/Pharmaceutical Compositions/Formulations

In some embodiments, provided is a composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, as described herein.

In some embodiments, provided is a composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in a composition is in the designated salt, solid form, crystalline form, or crystalline salt form.

In some embodiments, provided is a composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 85% of Compound I present in a composition is in the designated salt, solid form, crystalline form, or crystalline salt form.

In some embodiments, provided is a composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 90% of Compound I present in a composition is in the designated salt, solid form, crystalline form, or crystalline salt form.

In some embodiments, provided is a composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 95% of Compound I present in a composition is in the designated salt, solid form, crystalline form, or crystalline salt form.

In some embodiments, provided is a composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 99% of Compound I present in a composition is in the designated salt, solid form, crystalline form, or crystalline salt form.

In some embodiments, provided is a pharmaceutical composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 85% of Compound I present in a pharmaceutical composition is in the designated salt, solid form, crystalline form, or crystalline salt form.

In some embodiments, provided is a pharmaceutical composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 90% of Compound I present in a pharmaceutical composition is in the designated salt, solid form, crystalline form, or crystalline salt form.

In some embodiments, provided is a pharmaceutical composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 95% of Compound I present in a pharmaceutical composition is in the designated salt, solid form, crystalline form, or crystalline salt form.

In some embodiments, provided is a pharmaceutical composition comprising a salt or solid form of 6-fluoro-5-[4-({6-fluoro-4-oxo-2H,3H,5H-furo[3,2-c]quinolin-7-yl} methyl) piperazin-1-yl]-N-methylpyridine-2-carboxamide (Compound I), or a salt or solvate thereof, wherein at least 99% of Compound I present in a pharmaceutical composition is in the designated salt, solid form, crystalline form, or crystalline salt form.

In some embodiments, Compound I, or a pharmaceutically acceptable salt or solvate thereof, is administered to a subject in need thereof, either alone or in combination with pharmaceutically acceptable carriers, excipients, or diluents, in a pharmaceutical composition, according to standard pharmaceutical practice. In one embodiment, Compound I, or a pharmaceutically acceptable salt or solvate thereof, may be administered to animals. Compound I, or a pharmaceutically acceptable salt or solvate thereof, can be administered orally or parenterally, including the intravenous, intramuscular, intraperitoneal, subcutaneous, rectal, and topical routes of administration.

In another aspect, provided herein are pharmaceutical compositions comprising Compound I or a pharmaceutically acceptable salt or solvate thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating cancer using Compound I or a pharmaceutically acceptable salt or solvate thereof, in combination with an additional therapeutic agent.

In some embodiments, the additional therapeutic agent is an anticancer agent.

In some embodiments, the additional therapeutic agent is administered at the same time as Compound I or a pharmaceutically acceptable salt or solvate thereof. In some embodiments, the additional therapeutic agent and Compound I or a pharmaceutically acceptable salt or solvate thereof, are administered sequentially. In some embodiments, the additional therapeutic agent is administered less frequently than Compound I or a pharmaceutically acceptable salt or solvate thereof. In some embodiments, the additional therapeutic agent is administered more frequently than Compound I or a pharmaceutically acceptable salt or solvate thereof. In some embodiments, the additional therapeutic agent is administered prior to the administration of Compound I or a pharmaceutically acceptable salt or solvate thereof. In some embodiments, the additional therapeutic agent is administered after the administration of Compound I or a pharmaceutically acceptable salt or solvate thereof.

### EXAMPLES

### Abbreviations

### Chemical Abbreviation

ACN Acetonitrile
DCM Dichloromethane
DMSO Dimethyl sulfoxide
EtOH Ethanol
EtOAc Ethyl acetate
IPA Isopropanol
IPAc Isopropyl acetate
DIPEA N,N-diisopropylethylamine
DMF N,N-Dimethylformamide
MeOH Methanol
MIBK Methyl isobutyl ketone
MTBE tert-Butyl methyl ether
NMP N-Methylpyrrolidone
PTSA p-Toluenesulfonic acid
THF Tetrahydrofuran
2-MeTHF 2-Methyltetrahydrofuran
TFA Trifluoroacetic acid
MEK 2-Butanone

### Other Abbreviations (Alphabetical Order)

d Day
DSC Differential Scanning Calorimetry
DVS Dynamic Vapor Sorption
eq. equivalent
FaSSIF Fasted State Simulated Intestinal Fluid
FeSSIF Fed State Simulated Intestinal Fluid
h Hour
HPLC High Performance Liquid Chromatography
LC Low crystallinity
¹H-NMR Proton Nuclear Magnetic Resonance
NMR Nuclear Magnetic Resonance
min Minute
NP New pattern
pk peak
RT Room temperature (20-25° C.)
SGF Simulated Gastric Fluid
TGA Thermogravimetric Analysis
XRPD X-ray Powder Diffraction

### Example 1: Syntheses of Compound I Form I

### Synthesis 1

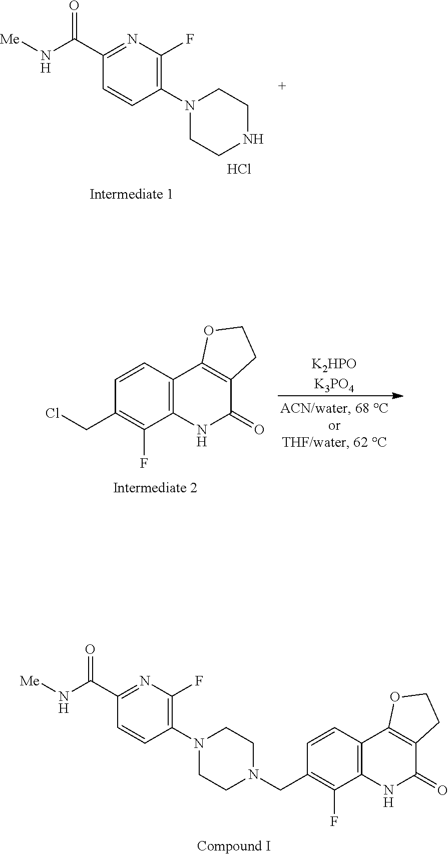 [SMILES: CNC(=O)c1ccc(N2CCNCC2)c(F)n1 ; Cl ; O=c1[nH]c2c(F)c(CCl)ccc2c2c1CCO2 ; "K2HPOK3PO4" ; CNC(=O)c1ccc(N2CCN(Cc3ccc4c5c(c(=O)[nH]c4c3F)CCO5)CC2)c(F)n1]

Intermediate 1 and Intermediate 2 can be made according to methods known in the art, such as those described in WO 2023/212219.

Compound I Form I can be prepared according to the following procedure.

In a reaction vessel, dipotassium phosphate (about 0.9×), tripotassium phosphate (about 1.7×), and water (about 9V) were combined and mixed well until all solids dissolved. To a reactor was charged Intermediate 2 (X), Intermediate 1 (about 1.5×) and acetonitrile (about 19V). The contents were adjusted to around 68° C.; then, the phosphate solution was charged in a drop-wise manner. The contents were agitated at around 68° C. for about 1 day until the reaction is deemed complete. The contents were adjusted to around 22° C. and filtered. The solids were washed with a mixture of 1:3 v/v ACN/water (about 12V), followed by ACN (about 5V). The solids were dried in a vacuum oven at about 50° C. and with a N₂ sweep for about one day, to obtain Compound I Form I.

Compound I Form I can also be prepared according to the following procedure.

In a reaction vessel, dipotassium phosphate (about 0.8×), tripotassium phosphate (about 1.5×), and water (about 6V) were combined and mixed well until all solids dissolved. To a reactor was charged Intermediate 2 (X), Intermediate 1 (about 1.2×) and THF (about 18V). The contents were adjusted to around 62° C.; then, the phosphate solution was charged in a drop-wise manner. The contents were agitated at around 62° C. for about 1 day until the reaction is deemed complete. The contents were adjusted to about 5° C. and filtered. The solids were washed with a mixture of 1:4 v/v THF/water (about 10V), followed by ACN (about 4V). The solids were dried in a vacuum oven at about 50° C. and with a N₂ sweep for about 1 day, to obtain Compound I Form I.

### Synthesis 2

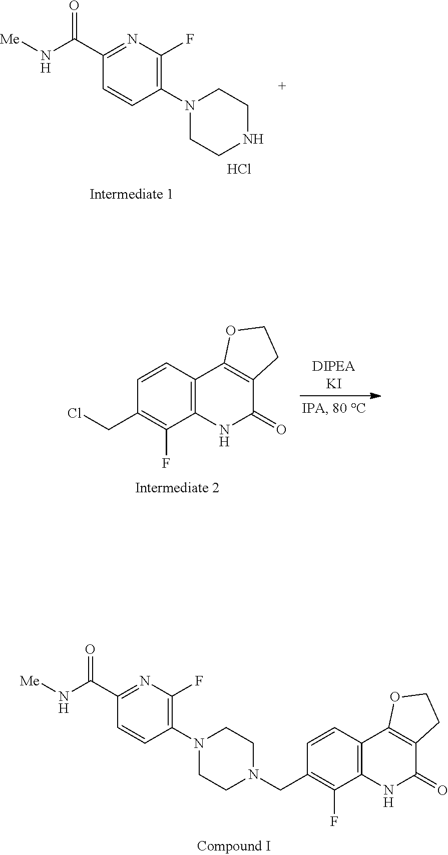 [SMILES: CNC(=O)c1ccc(N2CCNCC2)c(F)n1 ; Cl ; O=c1[nH]c2c(F)c(CCl)ccc2c2c1CCO2 ; "DIPEAKI" ; CNC(=O)c1ccc(N2CCN(Cc3ccc4c5c(c(=O)[nH]c4c3F)CCO5)CC2)c(F)n1]

Compound I Form I can also be prepared according to the following procedure.

To a reactor was charged Intermediate 1 (about 1.2×), Intermediate 2 (X), KI (about 0.07×), IPA (about 30V) and DIPEA (about 2×). The contents were adjusted to about 80° C. and agitated for about 1 day until the reaction is deemed complete. The contents were filtered, and the solids were rinsed with IPA (about 5V). A slurry wash of the solids in 1:1 v/v IPA/water (about 22V) at around 80° C. was performed. The contents were adjusted to around 22° C. and filtered. The solids were rinsed with IPA (about 5V) and dried in a vacuum oven at around 50° C. and with a N₂ sweep for about 1 day, to obtain Compound I Form I.

### Example 2: Free Base Polymorph Screen

### Analysis Methods

### XRPD

XRPD diffractograms were collected with an X-ray diffractometer. The sample was prepared on a zero-background silicon wafer by gently pressing onto the flat surface. The parameters of XRPD diffraction are given in Table 1.

TABLE 1
Parameters for XRPD Testing

| Instrument | Bruker, D8 Advance | |
|---|---|---|
| Radiation | Cu Ka (l = 1.5418 Å) | |
| Detector | LynxEye | |
| Scan angle | 3-40° | (2q) |
| Scan step | 0.013° | (2q) |
| Scan speed | 0.1 | s/step |
| Tube voltage/current | 40 kV/40 mA | |
| Divergence slit | 0.6 | mm |

| | |
|---|---|
| Rotation | On |
| Sample holder | Zero-background sample pan |

### TGA

TGA analysis was performed using a TA Instrument. About 1-5 mg of a sample was loaded onto a pre-tared aluminum pan and heated with the parameters in Table 2. The data was analyzed using TRIOS.

TABLE 2
Parameters of TGA Testing

| Instrument | TA TGA 5500 | |
|---|---|---|
| Sample pan | Aluminum, open | |
| Temperature range | RT-300° | C. |
| Heating rate | 10° | C./min |

| | |
|---|---|
| Purge gas | N2 |
| Flow rate | Balance chamber: 40 mL/min |
| | Sample chamber: 60 mL/min |

### DSC

DSC analysis was performed with a TA Instrument. About 1-3 mg of a sample was placed into an aluminum pan with pin-hole and heated with the parameters in Table 3. The data was analyzed using TRIOS.

TABLE 3
Parameters of DSC Analysis

| Instrument | TA DSC 2500/250 | |
|---|---|---|
| Sample pan | Aluminum, pin-holed | |
| Temperature range | 25-300° | C. |
| Heating rate | 10° | C./min |
| Purge gas | N₂ | |
| Flow rate | 50 | mL/min |

### DVS

Moisture sorption/desorption data were collected on a Vsorp Dynamic Moisture Sorption Analyzer. About 13 mg of a sample was placed into a tared sample chamber and automatically weighed. The samples were analyzed with the setting parameters in Table 4.

TABLE 4
Parameters of DVS Analysis

| Instrument | SMS, DVS Intrinsic | |
|---|---|---|
| dm/dt | 0.002%/min | |
| Drying/Measurement temperature | 40° C./25° C. | |
| Cycle | Full cycle | |
| Save data rate | 5 | s |
| Total flow rate | 200 | ccm |
| Post experiment total flow | 200 | ccm |
| Minimum dm/dt stability duration | 30 | min |
| Maximum dm/dt equilibrium time | 120 | min |

| | |
|---|---|
| Method | Adsorption: 0, 10, 20, 30, |
| | 40, 50, 60, 70, 80, 90 |
| | Desorption: 80, 70, 60, 50, |
| | 40, 30, 20, 10, 0 |

### HPLC

HPLC analysis was performed with an Agilent HPLC 1260 series instrument. HPLC method for solubility and stability testing is presented in Table 5.

TABLE 5
HPLC Method for Solubility and Stability Testing

| | |
|---|---|
| Instrument | Agilent 1260 HPLC series |
| Column | Ascentis Express C18, 4.6 × 100 mm, 2.7 μm |
| Mobile Phase | A: 0.05% TFA in water; B: 0.05% TFA in ACN |
| Gradient (T/B %) | 0.0/5%, 7/20%, 8/20%, 13/35%, |
| | 16/95%, 17/95%, 17.1/5% |
| Column Temperature | 30° C. |
| Detector | DAD; 220 nm |
| Flow Rate | 1 mL/min |
| Injection Volume | 2 or 3 mL |
| Run Time | 17.10 min |
| Post Time | 3 min |
| Diluent | MeOH/water (v/v, 9:1) |

### ¹H-NMR

¹H-NMR spectra were collected on a Bruker 400 MHz instrument. Unless specified, samples were prepared in DMSO-d6 solvent and measured with the parameters in Table 6. The data was analyzed using MestReNova.

TABLE 6
Parameters for ¹H-NMR Analysis

| Instrument | Bruker | |
|---|---|---|
| Frequency | 400 | MHz |
| Scan times | 4 | |
| Temperature | 295 | K |
| Relaxation delay | 1 | s |

### Summary

In this study, the objective was to conduct a polymorph screen for the free base of Compound I.

Polymorph screening was carried out using solvents and various crystallization methods, including slurry conversion, cooling, evaporation, anti-solvent precipitation and cyclic heating-cooling. 14 crystal forms were found, including four anhydrates of Compound I Forms I, III, IV and VII. Compound I Form I has the highest melting point, and other polymorphs converted into Compound I Form I after thermal treatment by DSC.

After slurry at RT, Compound I Form IV converted to Compound I Form III, and Compound I Form VII converted to Compound I Form I. Competitive slurries showed that Compound I Form I is more stable above 80° C., and Compound I Form III more stable below 50° C.

### Characterization and Definition of Crystal Forms

A total of 14 crystal forms, Compound I Forms I-XIV, were identified. Among them, Compound I Forms I, III, IV and VII are anhydrates.

### Compound I Form I

Compound I Form I, which can be prepared according to methods described herein, was characterized by XRPD, DSC and TGA. The sample showed fine crystals with slight aggregation. XRPD showed the material was highly crystalline (FIG. 1A). Negligible organic solvent was detected by¹H-NMR. TGA (FIG. 1B) showed 0.5% weight loss before 65° C. DSC (FIG. 1B) showed a sharp endothermic peak at 285° C. (onset), due to melting. The sample was identified as an anhydrate and assigned as Compound I Form I.

Compound I Form I was used to obtain the other freebase forms.

### Compound I Form II

Compound I Form II was obtained by anti-solvent precipitation in MeOH/DCM (1/4, v/v) with toluene as anti-solvent, which was characterized by XRPD, TGA, DSC and ¹H-NMR. The characterization data are shown in FIG. 2A and FIG. 2B.

XRPD (FIG. 2A) showed Compound I Form II exhibited high crystallinity and fine crystals with aggregation. Thermal analysis (FIG. 2B) showed 7.6% weight loss at 115-185° C. in TGA and two endothermic peaks at 159 and 287° C. (onset) in DSC, due to desolvation and melting. ¹H-NMR showed 7.9% toluene. Therefore, Compound I Form II is a solvate of toluene. After heating to 200° C., Compound I Form II converted to Compound I Form I. Compound I Form II can also be prepared in n-butanol/anisole.

### Compound I Form III

Compound I Form III was obtained by anti-solvent precipitation in NMP with IPAc as anti-solvent, which was characterized by XRPD, TGA, DSC and ¹H-NMR.

XRPD (data not shown) showed Compound I Form III exhibited high crystallinity and fine crystals with aggregation. Thermal analysis showed 1.4% weight loss at 125-295° C. in TGA and one endothermic peak at 287° C. (onset) in DSC, due to melting. Small thermal signals around 270° C. were found. ¹H-NMR showed 0.2% residual IPAc. Therefore, Compound I Form III is an anhydrate. Compound I Form IV

Compound I Form IV was obtained by anti-solvent precipitation in acetic acid with IPA as anti-solvent, which was characterized by XRPD, TGA, DSC and ¹H-NMR. The characterization data are shown in FIG. 4A and FIG. 4B.

XRPD (FIG. 4A) showed Compound I Form IV exhibited moderate crystallinity and fine crystals with aggregation. Thermal analysis (FIG. 4B) showed 0.7% weight loss at 255-285° C. in TGA, and two endothermic peaks at 272 and 286° C. and one exothermic peak at 276° C. (onset) in DSC, due to melting recrystallization. ¹H-NMR showed 0.9% IPA and 0.4% CH₃COOH. Therefore, Compound I Form IV is an anhydrate. Compound I Form IV converted to Compound I Form III after slurry in ACN/H₂O (6/1) for 1 day at RT and converted to Compound I Form I after heating to 277° C.

### Compound I Form V

Compound I Form V was obtained by slurry in DMSO at RT, which was characterized by XRPD, TGA, DSC and ¹H-NMR. The characterization data are shown in FIG. 5A and FIG. 5B.

XRPD (FIG. 5A) showed Compound I Form V exhibited high crystallinity and fine crystals with aggregation. Thermal analysis (FIG. 5B) showed 12.1% weight loss at 115-155° C. in TGA, and two endothermic peaks at 122 and 287° C. (onset) in DSC, due to desolvation and melting. ¹H-NMR showed 14.2% DMSO. Therefore, Compound I Form V is a solvate of DMSO. After heating to 200° C., Compound I Form V converted to Compound I Form I.

### Compound I Form VI

Compound I Form VI was obtained by slow evaporation in MeOH, which was characterized by XRPD, TGA, DSC and ¹H-NMR. The characterization data are shown in FIG. 6A and FIG. 6B.

XRPD (FIG. 6A) showed Compound I Form VI exhibited high crystallinity and rod-shaped crystals. Thermal analysis (FIG. 6) showed 2.6% weight loss before 85° C. and 1.7% weight loss at 180-290° C. in TGA, and two endothermic peaks at 26 and 285° C. and one small exothermic peak at 213° C. (onset) in DSC, due to dehydration, melting and phase transition. ¹H-NMR showed negligible organic solvent. Therefore, Compound I Form VI is a hydrate. After vacuum drying at 40° C., Compound I Form VI converted to Compound I Form VII, while it converted to Compound I Form I after heating to 220° C.

### Compound I Form VII

Compound I Form VII was obtained by slow evaporation in MeOH followed by drying at about 40° C. for about 3 h under vacuum, which was characterized by XRPD, TGA and DSC. The characterization data are shown in FIG. 7A and FIG. 7B.

XRPD (FIG. 7A) showed Compound I Form VII exhibited high crystallinity and rod-shaped crystals. Thermal analysis (FIG. 7B) showed 1.2% weight loss at 190-290° C. in TGA, and one endothermic peak at 284° C. (onset) in DSC, due to melting. Therefore, Compound I Form VII is an anhydrate. After slurry in ACN/H₂O (6/1) at RT for 1 day, Compound I Form VII converted to Compound I Form I.

### Compound I Form VIII

Compound I Form VIII was obtained by repetition trial of anti-solvent precipitation in acetic acid with MTBE as anti-solvent, which was characterized by XRPD, TGA, DSC and ¹H-NMR. The characterization data are shown in FIG. 8A and FIG. 8B.

XRPD (FIG. 8A) showed Compound I Form VIII exhibited moderate crystallinity and fine crystals with aggregation. Thermal analysis (FIG. 8B) showed 8.1% weight loss at 100-145° C. and 0.1% weight loss at 265-290° C. in TGA, and two endothermic peak at 109 and 284° C. (onset) in DSC, due to desolvation and melting. ¹H-NMR showed 7.3% acetic acid and 0.1% MTBE. Therefore, Compound I Form VIII is a solvate of acetic acid. After heating to 200° C., Compound I Form VIII converted to Compound I Form I with minor Compound I Form IV.

### Compound I Form IX

Compound I Form IX was obtained by repetition trial of anti-solvent precipitation in acetic acid with IPA as anti-solvent, which was characterized by XRPD, TGA, DSC and ¹H-NMR. The characterization data are shown in FIG. 9A and FIG. 9B.

XRPD (FIG. 9A) showed Compound I Form IX exhibited low crystallinity and fine crystals with aggregation. Thermal analysis (FIG. 9B) showed 3.4% weight loss at 70-160° C., 0.5% weight loss at 160-225° C. and 0.5% weight loss at 225-290° C. in TGA, and three endothermic peaks at 102, 257 and 286° C. and one exothermic peak at 270° C. (onset) in DSC, due to desolvation, phase transition and melting. ¹H-NMR showed 3.6% acetic acid and 0.7% IPA. Therefore, Compound I Form IX is a solvate of acetic acid. Compound I Form IX converted to Compound I Form IV after heating to 215° C. and converted to Compound I Form I after heating to 270° C.

### Compound I Form X

Compound I Form X was obtained by cooling in acetone/MeOH, but limited sample was obtained. A mixture of Compound I Form I and X was obtained by cyclic heating-cooling in THF/water. The suspension was divided into two parts for slurry experiments. Half of the suspension was stirred at RT and the other half was stirred at about 50° C. After 4 days, Compound I Form I was obtained by slurry at both RT and about 50° C. The XRPD is shown in FIG. 10A.

### Compound I Form XI

Compound I Form XI was obtained by repetition trial of anti-solvent precipitation in MeOH/DCM with IPA as anti-solvent, which was characterized by XRPD, TGA, DSC and ¹H-NMR. The characterization data are shown in FIG. 11A and FIG. 11B.

XRPD (FIG. 11A) showed Compound I Form XI exhibited moderate crystallinity and fine crystals with aggregation. Thermal analysis (FIG. 11B) showed 4.8% weight loss at 115-170° C. in TGA, and two endothermic peaks at 128 and 287° C. (onset) in DSC, due to desolvation and melting. ¹H-NMR showed 4.9% IPA and 0.4% DCM. Therefore, Compound I Form XI is a solvate of IPA. After heating to 200° C., Compound I Form XI converted to Compound I Form I.

### Compound I Form XII

Compound I Form XII was obtained by anti-solvent precipitation in MeOH/DCM with MIBK as anti-solvent at RT for 1 h, which was characterized by XRPD, TGA, DSC and ¹H-NMR. The characterization data are shown in FIG. 12A and FIG. 12B.

XRPD (FIG. 12A) showed Compound I Form XII exhibited high crystallinity. Thermal analysis (FIG. 12B) showed negligible weight loss before melting in TGA, and one endothermic peak at 286° C. (onset) in DSC, due to melting. Compound I Form XII converted to Compound I Form XIII after extended slurry for 1 d.

### Compound I Form XIII

Compound I Form XIII was obtained by anti-solvent precipitation in MeOH/DCM with MIBK as anti-solvent at RT for 1 day, which was characterized by XRPD, TGA, DSC and ¹H-NMR. The characterization data are shown in FIG. 13A and FIG. 13B.

XRPD (FIG. 13A) showed Compound I Form XIII exhibited high crystallinity and fine crystals with aggregation. Thermal analysis (FIG. 13B) showed 5.6% weight loss at 100-160° C. in TGA, and two endothermic peaks at 121 and 287° C. (onset) in DSC, due to desolvation and melting. ¹H-NMR showed 5.0% MIBK. Therefore, Compound I Form XIII is a solvate of MIBK. After heating to 200° C., Compound I Form XIII converted to Compound I Form I.

### Compound I Form XIV

Compound I Form XIV was obtained by repetition trial of anti-solvent precipitation in MeOH/DCM with MIBK as anti-solvent. After drying at about 40° C. overnight under vacuum, Compound I Form XIV mostly converted to Compound I Form I, and after extended slurry for about 1 day, Compound I Form XIV converted to Compound I Form III. The XRPD is shown in FIG. 14A.

### Stability Relationship Study of Compound I Forms I and III

Four anhydrates of Compound I Forms I, III, IV and VII were identified. After slurry in ACN/water (6/1) for 1 day, Compound I Form IV converted to Compound I Form III, and Compound I Form VII converted to Compound I Form I. Competition slurry was conducted between the two more stable anhydrates of Compound I Forms I and III.

About 5 mg each of Compound I Form I and Form III were suspended in 0.5 mL solvent at desired temperatures which was pre-saturated by Compound I Form I. The sample was withdrawn for XRPD test at different time points. The results are summarized in Table 7. Compound I Form I is more stable at 80° C., and Compound I Form III more stable at RT-50° C. The conversion rate at RT-50° C. was slow.

TABLE 7
Results of Competition Slurry of Compound I Forms I and III

| | | XRPD Result | | |
|---|---|---|---|---|
| Solvent (v/v) | Temperature | 1 d | 7 d | 14 d |
| EtOH | RT | Compound I | Compound I Form | Compound I Form |
| | | Form I + III | III + I | III + I |
| EtOH | 50° C. | Compound I | Compound I Form | Compound I Form |
| | | Form I + III | III + I | III + I |
| ACN/Water (6/1) | RT | Compound I | Compound I Form | Compound I Form |
| | | Form I + III | III + I | III + I (trace) |
| ACN/Water (6/1) | 50° C. | Compound I | Compound I Form | Compound I Form |
| | | Form I + III | III + I (trace) | III |
| ACN/Water (6/1) | 80° C. | Compound I | / | / |
| | | Form I | | |
| NMP | 50° C. | Compound I | / | / |
| | | Form I + III | | |

### Additional Characterization of Compound I Form III

The preparation procedures of Compound I Form III are described in Table 8. Seeds of Compound I Form III as described in the table were prepared as follows: 50 mg of Compound I Form I was dissolved in 1.5 mL MeOH/DCM (1/4, v/v) at room temperature. The solution was filtered, and MIBK was added into the filtrate dropwise until solid precipitated out. The sample was collected by filtration, and the seeds were obtained by drying at 40° C. overnight under vacuum.

The sample was characterized by XPRD, DSC, TGA, DVS and ¹H-NMR. The characterization results are shown in FIG. 3A, FIG. 3B, and FIG. 3C.

The XRPD pattern (FIG. 3A) was consistent with Compound I Form III. The sample showed fine crystals with aggregation under microscopy. Thermal analysis (FIG. 3B) showed the sample had 1.2% weight loss at 240-290° C. in TGA, and two endothermic peaks at 268 and 281° C. and one exothermic peak at 271° C. (onset) in DSC, due to melting recrystallization. ¹H-NMR showed 1.3% NMP and 0.2% IPAc. After heating to 272° C., Compound I Form III converted to Compound I Form I. DVS result (FIG. 3C) showed Compound I Form III was slightly hygroscopic with water uptake of 0.77/1.13% at 80/90% RH, and the crystal form remained unchanged after DVS testing.

TABLE 8
Scale-up of Compound I Form III

| No. | Procedure | Result |
|---|---|---|
| 1 | About 500 mg of Compound I Form I was dissolved in 30 mL NMP at | Clear |
| | about 80° C. for 1.5 h. The solution was filtered and cooled to about 50° C. | |
| | Compound I Form III seed, which was made according to methods | |
| | described herein, was added and stirred at about 50° C. overnight. | |
| 2 | 30 mL IPAc was added into the clear solution and the system remained | Suspension |
| | clear. Compound I Form III seed, which was made according to methods | |
| | described herein, was added. The suspension was stirred at about 50° C. for | |
| | about 2 h and at RT for about 4 h. | |
| 3 | 30 mL IPAc was added into the suspension and stirred for about 2 h at RT. | Compound I |
| | Most of the suspension (about 65 mL) was filtered and the filter cake was | Form III |
| | dried at about 40° C. overnight under vacuum. | |
| 4 | The filtrate and residual suspension were mixed and stirred at RT for | Compound I |
| | about 12 h. Then 30 mL MTBE was added and stirred for about 8 h at RT. | Form III |
| | The suspension was filtered and dried at about 40° C. overnight under | |
| | vacuum. | |

### Evaluation of Compound I Form III

### Mechanical Stability of Compound I Form III

Appropriate amount of Compound I Form III was manually ground by pestle and mortar for about 2 and 5 minutes, respectively. The ground samples were analyzed by XRPD. The crystal form of Compound I Form III remained unchanged with crystallinity decrease after grinding, indicating likely acceptable mechanical stability.

### Solid-State Stability Study

About 8 mg of Compound I Form III were put at 40° C./75% RH (open) and 60° C. (capped) conditions for 7 days. Then the stability sample was tested by XRPD and HPLC to check the physical and chemical stability. The results are presented in Table 9 and Table 10.

The crystal form of Compound I Form III remained at both conditions.

TABLE 9
Results of Solid-state Stability Study

| Solid | Purity (area %) @7 D | | | XRPD @7 D | |
|---|---|---|---|---|---|
| Form | Initial | 40° C./75% RH | 60° C. | 40° C./75% RH | 60° C. |

TABLE 10
Impurity Table of Compound I Form
III in Solid-state Stability Study

| | | Purity | Impurity (area %) | |
|---|---|---|---|---|
| Solid Form | Condition | (area %) | RRT 0.95 | RRT 1.07 |
| Compound I | Initial | 96.62 | 0.29 | 1.41 |
| Form III | 40° C./75% RH | 96.13 | 0.34 | 1.86 |
| | 60° C. | 96.29 | 0.37 | 1.61 |

The solid-state properties of Compound I Form I and Compound I Form III are summarized in Table 11.

TABLE 11
Solid-state Properties of Compound I Form I and Compound I Form III

| | Compound I Form I | Compound I Form III |
|---|---|---|
| Solid Form | Anhydrate | Anhydrate |
| Solid-state Stability* | Physically and chemically stable | Physically stable for |
| @60° C. and 40° C./75% RH | for at least 1 week | at least 1 week |
| Mechanical Stability | / | No form change, crystallinity |
| under Manual Grinding | | decrease |

*The solubility and stability results of Compound I Form I refer to the study described in Example 3.

### Polymorph Screening

Solvents used for the polymorph screening are given in Table 12.

TABLE 12
List of Solvents

| # | Solvent |
|---|---|
| 1 | MeOH |
| 2 | EtOH |
| 3 | DMF |
| 4 | IPA |
| 5 | Acetone |
| 6 | MEK |
| 7 | ACN |
| 8 | EtOAc |
| 9 | IPAc |
| 10 | Acetic acid |
| 11 | THF |
| 12 | DCM |
| 13 | Water |
| 14 | MTBE |
| 15 | DMSO |
| 16 | NMP |
| 17 | MIBK |
| 18 | Toluene |

Polymorph screening experiments are summarized in Table 13. 14 crystalline forms were found, and Compound I Forms I, III, IV and VII are anhydrates.

TABLE 13
Summary of Polymorph Screening

| No. | Method | Crystalline Form |
|---|---|---|
| 1 | Slurry conversion | Compound I Forms I-III, V |
| 2 | Anti-solvent precipitation | Compound I Form I-IV, VIII, |
| | | IX, XI-XIV |
| 3 | Evaporation crystallization | Compound I Form I, VI, VII |
| 4 | Cooling crystallization | Compound I Form I, V, VI, X |
| 5 | Cyclic heating-cooling | Compound I Form I, II, X |

| | |
|---|---|
| Total | Compound I Forms I-XIV |

### Slurry Conversion

Appropriate amount of Compound I Form I was added into different solvents to make suspensions which were kept stirring at RT and 50° C. for 4 and 7 days and at 80° C. for 4 days. Solid samples were collected by filtration and analyzed by XRPD.

The results are summarized in Table 14-Table 16. Compound I Form I remained unchanged at most conditions. Compound I Form II appeared after slurry in butanol/anisole (1/1) at 80° C., Compound I Form III appeared after slurry in acetic acid/water (1/100) at RT, and pure Compound I Form V was obtained by slurry in DMSO at RT and 50° C.

TABLE 14
Results of Slurry at RT

| | Solvent | XRPD Result | |
|---|---|---|---|
| No. | (v/v, Vol.) | Day 4 | Day 7 |
| 1 | MeOH (50 V) | Compound I Form | Compound I Form |
| | | I | I |
| 2 | EtOH (50 V) | Compound I Form | Compound I Form |
| | | I | I |
| 3 | Acetone (50 V) | Compound I Form | Compound I Form |
| | | I | I |
| 4 | EtOAc (50 V) | Compound I Form | Compound I Form |
| | | I | I |
| 5 | THF (50 V) | Compound I Form | Compound I Form |
| | | I | I |
| 6 | NMP (15 V) | Compound I Form | Compound I Form |
| | | I | I |
| 7 | Water (50 V) | Compound I Form | Compound I Form |
| | | I | I |
| 8 | DMSO (25 V) | Compound I | Compound I |
| | | Form V | Form V |
| 9 | DMF (25 V) | Compound I Form | Compound I Form |
| | | I | I |
| 10 | THF/Water | Compound I Form | Compound I Form |
| | (9/1, 50 V) | I | I |
| 11 | ACN/Water | Compound I Form | Compound I Form |
| | (6/1, 50 V) | I | I |
| 12 | Acetone/Water | Compound I Form | Compound I Form |
| | (9/1, 50 V) | I | I |
| 13 | MeOH/Water | Compound I Form | Compound I Form |
| | (9/1, 50 V) | I | I |
| 14 | DMSO/Water | Compound I Form | Compound I Form |
| | (1/1, 50 V) | I | I |
| 15 | Acetic acid/Water | Compound I Form | Compound I Form |
| | (1/100, 50 V) | I + III | I + III |
| 16 | ACN (50 V) | Compound I Form | Compound I Form |
| | | I | I |
| 17 | Acetic acid/EtOH | Compound I Form | Compound I Form |
| | (1/50, 50 V) | I | I |

TABLE 15
Results of Slurry at 50° C.

| | Solvent | XRPD Result | |
|---|---|---|---|
| No. | (v/v, Vol.) | Day 4 | Day 7 |
| 1 | MeOH (50 V) | Compound I Form I | Compound I |
| | | | Form I |
| 2 | EtOH (50 V) | Compound I Form I | Compound I |
| | | | Form I |
| 3 | Acetone (50 V) | Compound I Form I | Compound I |
| | | | Form I |
| 4 | EtOAc (50 V) | Compound I Form I | Compound I |
| | | | Form I |
| 5 | ACN (50 V) | Compound I Form I | Compound I |
| | | | Form I |
| 6 | THF (50 V) | Compound I Form I | Compound I |
| | | | Form I |
| 7 | NMP (15 V) | Compound I Form I | Compound I |
| | | | Form I |
| 8 | Water (50 V) | Compound I Form I | Compound I |
| | | | Form I |
| 9 | DMSO (25 V) | Compound I Form V | Compound I |
| | | | Form V |
| 10 | DMF (25 V) | Compound I Form I | Compound I |
| | | | Form I |
| 11 | THF/Water (9/1, 50 V) | Compound I Form I | Compound I |
| | | | Form I |
| 12 | ACN/Water (6/1, 50 V) | Compound I Form I | Compound I |
| | | | Form I |
| 13 | Acetone/Water (9/1, 50 V) | Compound I Form I | Compound I |
| | | | Form I |
| 14 | MeOH/Water (9/1, 50 V) | Compound I Form I | Compound I |
| | | | Form I |
| 15 | DMSO/Water (1/1, 50 V) | Compound I Form I | Compound I |
| | | | Form I |
| 16 | Aceticacid/ | Compound I Form I | Compound I |
| | Water(1/100, 50 V) | | Form I |
| 17 | Acetic acid/EtOH | Compound I Form I | Compound I |
| | (1/50, 50 V) | | Form I |

TABLE 16
Results of Slurry at 80° C.

| No. | Solvent (v/v, Vol.) | XRPD Result (4 d) |
|---|---|---|
| 1 | ACN (50 V) | Compound I Form I |
| 2 | DMF (25 V) | Compound I Form I |
| 3 | DMSO (25 V) | Compound I Form I |
| 4 | IPA (50 V) | Compound I Form I |
| 5 | Water (50 V) | Compound I Form I |
| 6 | ACN/Water (6/1, 50 V) | Compound I Form I |
| 7 | DMSO/Water (1/1, 50 V) | Compound I Form I |
| 8 | Acetic acid/IPAc (1/100, 50 V) | Compound I Form I |
| 9 | NMP/MIBK (1/10, 50 V) | Compound I Form I |
| 10 | Butanol/anisole (1/1, 50 V) | Compound I Form I + II |

### Cooling Crystallization

Cooling crystallization was performed in eleven selected solvents. About 15 mg of Compound I Form I was weighed into a glass vial, and then selected solvent was added to make a suspension with stirring at elevated temperature for about 20 m. Then the suspension was filtered to obtain saturated drug solution at elevated temperature. The filtrate was placed at RT first; if no solid occurred, the filtrate was placed at 5° C. refrigerator for cooling crystallization. Any solid obtained was characterized by XRPD.

The results are summarized in Table 17. Compound I Form V was obtained in DMSO, Compound I Form X was obtained in acetone/MeOH (4/1), and Compound I Form VI appeared in MeOH and MeOH/water (9/1).

TABLE 17
Results of Cooling Crystallization

| Solvent | Elevated | Results | |
|---|---|---|---|
| (v/v, Vol.) | Temp. (° C.) | RT | 5° C. |
| MeOH (67 V) | 50 | Clear | Compound I |
| | | | Form I + VI |
| THF (67 V) | 75 | Compound I | / |
| | | Form I | |
| THF/Water (9/1, 67 V) | 75 | Compound I | / |
| | | Form I | |
| DMSO/Water (2/1, 67 V) | 75 | Clear | Clear |
| MeOH/Water (9/1, 67 V) | 50 | Clear | Compound I |
| | | | Form I + VI |
| ACN/Water (6/1, 67 V) | 75 | Clear | Clear |
| Acetic acid/Water | 75 | Clear | Clear |
| (1/100, 67 V) | | | |
| DMSO/ACN (1/2, 67 V) | 75 | Clear | Compound I |
| | | | Form I |
| DMSO (67 V) | 75 | Clear | Compound I |
| | | | Form V |
| DMF (67 V) | 75 | Clear | Compound I |
| | | | Form I |
| Acetone/MeOH (4/1, 67 V) | 50 | Clear | Compound I |
| | | | Form X |

### Evaporation Crystallization

Evaporation crystallization was performed in eight selected solvents. Clear solutions or suspensions of Compound I were prepared at RT or 50° C.; after filtration, the filtrate was covered with pin-hole film and placed at ambient condition for slow evaporation.

The results are summarized in Table 18. Compound I Form VI appeared in solvents containing MeOH and Compound I Form I with extra peaks were obtained in DCM, THF and DCM/MeOH.

TABLE 18
Results of Evaporation Crystallization

| Solvent (v/v, Vol.) | XRPD Result |
|---|---|
| MeOH/DCM (1/4, 50 V) | Compound I Form I + Compound I Form VI |
| THF/Water (9/1, 200 V) | Compound I Form I + extra peaks |
| ACN/water (6/1, 200 V) | Compound I Form I |
| Acetone/Water (9/1, 200 V) | Compound I Form I |
| MeOH/Water (9/1, 200 V) | Compound I Form I + Compound I Form VI |
| DCM (200 V) | Compound I Form I + extra peaks |
| THF (200 V) | Compound I Form I + extra peaks |
| MeOH (200 V) | Compound I Form VI |

### Anti-Solvent Precipitation

Anti-solvent precipitation was performed according to solubility data. Appropriate amount of Compound I Form I was weighed into 8 mL glass vials and then selected solvent was added to make nearly saturated solution. After filtration, anti-solvent was added into the filtrate gradually until solids precipitated out or 5 mL anti-solvent was reached at RT. If precipitation occurred, solids were isolated by filtration and characterized accordingly.

The results are summarized in Table 19. Compound I Form II was obtained in MeOH/DCM/toluene, and Compound I Form IV was obtained in acetic acid/IPA, acetic acid/MTBE and NMP/water. Compound I Forms XI, XII and XIII were obtained using MeOH/DCM solvent.

TABLE 19
Results of Anti-solvent Precipitation

| Solvent | Anti- | | Vₛₒₗᵥₑₙₜ/ | XRPD Result | |
|---|---|---|---|---|---|
| (Vol.) | solvent | Observation | Vₐₙₜᵢ | 1 h | 1 day |
| MeOH/DCM | IPA | Precipitation occurred | 1/2 | Compound I | Compound I |
| (1/4, 30 V) | | immediately | | Form XI | Form XI |
| | MIBK | Precipitation occurred | 3/4 | Compound I | Compound I |
| | | immediately | | Form XII | Form XIII |
| | IPAc | Precipitation occurred | 3/2 | Compound I | Compound I |
| | | immediately | | Form XIII | Form XIII |
| | ACN | Precipitation occurred | 3/2 | Compound I | Compound I |
| | | immediately | | Form XI | Form XI |
| | Toluene | Precipitation occurred | 3/50 | Compound I | Compound I |
| | | after slurry for 40 min | | Form II | Form II |
| | MTBE | Precipitation occurred | 3/4 | Compound I | Compound I |
| | | immediately | | Form XI | Form XI |
| NMP | IPA | Clear | 1/7 | Clear | Clear |
| (71 V) | MIBK | Clear | 1/7 | Clear | Clear |
| | IPAc | Clear | 1/7 | Clear | Compound I |
| | | | | | Form III |
| | ACN | Precipitation occurred | 1/7 | Compound I | Compound I |
| | | after slurry for 20 min | | Form I | Form I |
| | Toluene | Precipitation occurred | 1/7 | Compound I | Compound I |
| | | after slurry for 60 min | | Form I + extra | Form I + extra |
| | | | | peak | peak |
| | Water | Precipitation occurred | 1/7 | Compound I | Compound I |
| | | after slurry for 10 min | | Form IV | Form IV |
| | MTBE | Precipitation occurred | 1/7 | Compound I | Compound I |
| | | after slurry for 30 min | | Form I | Form I |
| Acetic acid | IPA | Precipitation occurred | 1/25 | Compound I | Compound I |
| (10 V) | | immediately | | Form IV | Form IV |
| | MIBK | Clear | 1/50 | Clear | Clear |
| | IPAc | Clear | 1/50 | Clear | Clear |
| | ACN | Clear | 1/50 | Clear | Clear |
| | Toluene | Clear | 1/50 | Clear | Clear |
| | Water | Clear | 1/50 | Clear | Clear |
| | MTBE | Precipitation occurred | 1/50 | Compound I | Compound I |
| | | after slurry for 5 min | | Form IV | Form IV |

Vₛₒₗᵥₑₙₜ/Vₐₙₜᵢ is the volume ratio of solvent to anti-solvent

### Cyclic Heating-Cooling

Cyclic heating-cooling experiments were carried out in Crystal 16 Parallel Crystallizer. About 10 mg of Compound I Form I was added into 1 mL solvents, and a heating-cooling program was performed:
1. Equilibrated first at 20° C., and heated to 75/100° C. at 0.2° C./min
2. Held at 75/100° C. for 1
3. Cooled to 20° C. at 0.2° C./min, and held for 1 h
4. Repeated the above procedures once

Turbidimeter from Crystal 16 recorded if the solids were completely dissolved during heating. The results are presented in Table 20. Compound I Form X appeared in THF/water (9/1) and Compound I Form II appeared in n-butanol/anisole (1/1).

TABLE 20
Results of Cyclic Heating-cooling

| | Hot | Temp. | |
|---|---|---|---|
| Solvent (v/v, Vol.) | Dissolution | (° C.) | XRPD Result |
| THF/water (9/1, 100 V) | Yes | 75 | Compound I |
| | | | Form I + X |
| EtOH/water (9/1, 100 V) | No | | Compound I |
| | | | Form I |
| MEK (100 V) | No | | Compound I |
| | | | Form I |
| ACN/water (6/1, 100 V) | No | | Compound I |
| | | | Form I |
| DMSO/Water (2/1, 100 V) | No | 100 | Compound I |
| | | | Form I |
| Acetic acid/Water | No | | Compound I |
| (1/100, 100 V) | | | Form I |
| n-Butanol/anisole | Yes | | Compound I |
| (1/1, 100 V) | | | Form I + II |
| NMP/Water (1/10, 100 V) | No | | Compound I |
| | | | Form I |

### Example 3: Salt Screen

### Analysis Method

### XRPD

XRPD diffractograms were collected with a Bruker, D8 Advance X-ray diffractometer. The sample was prepared on a zero-background silicon wafer by gently pressing onto the flat surface. The parameters of XRPD diffraction are given in Table 21.

TABLE 21
Parameters for XRPD Testing

| Instrument | Bruker, D8 Advance | |
|---|---|---|
| Radiation | Cu Ka (l = 1.5418 Å) | |
| Detector | LynxEye | |
| Scan angle | 3-40° | (2q) |
| Scan step | 0.013° | (2q) |
| Scan speed | 0.1 | s/step |
| Tube voltage/current | 40 kV/40 mA | |
| Divergence slit | 0.6 | mm |

| | |
|---|---|
| Rotation | On |
| Sample holder | Zero-background sample pan |

### TGA

TGA analysis was performed using a TA Instrument. About 1-3 mg of a sample was loaded onto a pre-tared aluminum pan and heated with the parameters in Table 22. The data was analyzed using TRIOS.

TABLE 22
Parameters for TGA Testing

| Instrument | TA, Discovery TGA 5500 | |
|---|---|---|
| Sample pan | Aluminum, open | |
| Temperature range | RT-300° | C. |
| Heating rate | 10° | C./min |

| | |
|---|---|
| Purge gas | N₂ |
| Flow rate | Balance chamber: 10 mL/min |
| | Sample chamber: 40 mL/min |

### DSC

DSC analysis was performed with a TA Instrument. About 1-3 mg of a sample was placed into an aluminum pan with pin hole and heated with the parameters in Table 23. The data was analyzed using TRIOS.

TABLE 23
Parameters for DSC Analysis

| Instrument | TA, Discovery DSC 2500/250 | |
|---|---|---|
| Sample pan | Aluminum, pin-holed | |
| Temperature range | 25-300° | C. |
| Heating rate | 10° | C./min |
| Purge gas | N₂ | |
| Flow rate | 50 | mL/min |

### DVS

Moisture sorption/desorption data were collected on a Vsorp Dynamic Moisture Sorption Analyzer. About 50-100 mg of a sample was placed into a tared sample chamber and automatically weighed. The samples were analyzed with the setting parameters in Table 24.

TABLE 24
Parameters for DVS Analysis

| Instrument | ProUmid GmbH & Co. KG | |
|---|---|---|
| Sample temperature | 25° C. | |
| Time between cycles | 10 min | |
| Minimum time per climate setting | 50 min | |
| Maximum time per climate setting | 120 min | |
| Weight limit | 100% | |
| Equilibrium condition | 0.01%/45 min | |
| Climate cycle #1 | 0%-0% | 1 step at 40° C. for 3 h |
| Climate cycle #2 | 0%-90% | 9 steps at 25° C. |
| Climate cycle #3 | 80%-0% | 8 steps at 25° C. |

| | |
|---|---|
| Adsorption | 0, 10, 20, 30, 40, 50, 60, 70, 80, 90 |
| Desorption | 80, 70, 60, 50, 40, 30, 20, 10, 0 |

### ¹H-NMR

¹H-NMR spectra were collected on a Bruker 400 MHz instrument. Unless specified, samples were prepared in DMSO-d6 solvent and measured with the parameters in Table 25. The data was analyzed using MestReNova.

TABLE 25
Parameters for ¹H-NMR Analysis

| Instrument | Bruker | |
|---|---|---|
| Frequency | 400 | MHz |
| Scan times | 4 | |
| Temperature | 295 | K |
| Relaxation delay | 1 | s |

### HPLC

HPLC analysis was performed with an Agilent HPLC 1260 series instrument. HPLC method for solubility and stability testing is presented in Table 26.

TABLE 26
HPLC Method for Solubility and Stability Testing

| | |
|---|---|
| Instrument | Agilent 1260 HPLC series |
| Column | Ascentis Express C18, 4.6 × 100 mm, 2.7 μm |
| Mobile Phase | A: 0.05% TFA in water; B: 0.05% TFA in ACN |
| Gradient (T/B %) | 0.0/5%, 7/20%, 8/20%, 13/35%, |
| | 16/95%, 17/95%, 17.1/5% |
| Column Temperature | 30° C. |
| Detector | DAD; 220 nm |
| Flow Rate | 1 mL/min |
| Injection Volume | 2 or 3 mL |
| Run Time | 17.10 min |
| Post Time | 3 min |
| Diluent | MeOH/water (v/v, 9:1) |

### IC

IC analysis was performed with a Thermo ICS-6000 instrument. IC method for anion is presented in Table 27.

TABLE 27
IC Method

| | |
|---|---|
| Instrument | Thermo ICS-6000 |
| Column | Dionex IonPac ™AG11-HC(4*250 mm) |
| Guard Column | Dionex IonPac ™AG11-HC(4*50 mm) |
| Suppressor | Dionx ASRS 300 4 mm |
| Workstation | Chromeleon Workstation |
| Collection | 5.0 Hz |
| Cell Temperature | 35.0° C. |
| EGC Concentration | 30 mm (EGC 500 KOH) |
| Suppressor Mode | External Mode |
| Suppressor Current | 75 mA |
| Column Temperature | 30.0° C. |
| Flow Rate | 1.0 mL/min |
| Gradient Elution | Isocratic elution |
| Run Time | 20 min |
| Injection Volume | 25 μL |
| Flow Rate of External Water | 1.5 mL/min |

### Summary

The objective of this study was to conduct a salt screen for the free base of Compound I.

Salt screening experiments were carried out with ten pharmaceutically acceptable acids. A total of ten crystalline salts were identified. Among them, Compound I Phosphate Form I and Compound I Fumarate Form I showed acceptable solid-state properties; and other salt forms were hydrates and/or showed multiple thermal events on DSC. Compound I Form I, an anhydrate, was compared with Compound I Phosphate Form I and Compound I Fumarate Form I. DVS result showed that Compound I Form I, Compound I Phosphate Form I and Compound I Fumarate Form I were all slightly hygroscopic. The three solid forms were all chemically and physically stable at both 60° C. (capped) and 40° C./75% RH (open) conditions for 9 days.

Overall, Compound I Form I, Compound I Fumarate Form I and Compound I Phosphate Form I all showed acceptable solid-state properties. Compound I Phosphate Form I showed some advantages in solubility improvement compared to Compound I Fumarate Form I.

### Reagents

A total of ten acids were used for the salt screening, as presented in Table 28.

TABLE 28
List of Acids

| No. | Acid |
|---|---|
| 1 | HCl |
| 2 | H₂SO₄ |
| 3 | Maleic acid |
| 4 | H₃PO₄ |
| 5 | L-Tartaric acid |
| 6 | Fumaric acid |
| 7 | Citric acid |
| 8 | Succinic acid |
| 9 | PTSA |
| 10 | Methanesulfonic acid |

The solvents used for the salt screening are given in Table 29.

TABLE 29
List of Solvents

| No. | Solvent |
|---|---|
| 1 | MeOH |
| 2 | EtOH |
| 3 | Acetone |
| 4 | THF |
| 5 | DCM |
| 6 | Water |
| 7 | NMP |
| 8 | 2-MeTHF |
| 9 | EtOAc |
| 10 | ACN |
| 11 | Toluene |

### Results and Discussion

### DVS Test

DVS test was performed on Compound I Phosphate Form I and Compound I Fumarate Form I to exclude hygroscopic salt, and compared with Compound I Form I. The results are shown in FIG. 1C, FIG. 15G, and FIG. 16F.

DVS results showed Compound I Form I, Compound I Phosphate Form I and Compound I Fumarate Form I were all slightly hygroscopic with 1.04%/1.19%, 0.70%/0.81% and 0.49%/0.58% water uptake at 80%/90% RH, respectively. All the three solid forms remained unchanged after DVS test.

### Solid-State Stability

Solid-state stability trials of Compound I Form I, Compound I Phosphate Form I and Compound I Fumarate Form I were conducted at 60° C. (capped) and 40° C./75% RH (open) for 9 days. Duplicated samples were prepared at each condition. The stability sample was dissolved in diluent to prepare solution at 0.4 mg/mL for HPLC purity analysis. Solid samples were analyzed by XRPD to check the crystal form. The results are summarized in Table 30.

The crystal forms of the two salts and freebase remained unchanged, and no purity decrease was observed. All three forms were physically and chemically stable at 60° C. (capped) and 40° C./75% RH (open) for 9 days.

TABLE 30
Stability Evaluation Results

| | Initial Purity | Purity (area %) - 9 d | | XRPD - 9 d | |
|---|---|---|---|---|---|
| Solid Form | (area %) | 40° C./75% RH | 60° C. | 40° C./75% RH | 60° C. |
| Compound I Form I | 97.59 | 97.56/97.61 | 97.52/97.55 | Unchanged | Unchanged |
| Compound I Phosphate | 98.76 | 98.74/98.72 | 98.76/98.75 | Unchanged | Unchanged |
| Form I | | | | | |
| Compound I Fumarate | 98.74 | 98.71/98.72 | 98.71/98.71 | Unchanged | Unchanged |
| Form I | | | | | |

### Salt Screening

Salts were prepared with ten acids in three solvent systems of EtOH, THF and acetone/water (19/1, v/v).

About 25 mg of Compound I Form I was suspended in selected solvent at RT. Then 1.1 eq. of acid was added into the suspension at RT for salt formation (liquid acid was pre-diluted in corresponding solvent and added into the suspension); for H₂SO₄, an additional 0.55 eq. of H₂SO₄ was added. The mixture was stirred at RT for 1 d. 0.5 m more solvent was added to dilute several thick systems at 16 h (noted as * in the table). If clear solution or oil was obtained, various crystallization methods were used to induce precipitation. Solids were collected by filtration or centrifugation, and dried under vacuum at 40 FC for about 14 h. Salt screening results are summarized in Table 31. The experimental details and characterization results are presented in the following parts.

TABLE 31
Summary of Salt Screening

| | Solvent (v/v) | | |
|---|---|---|---|
| Acid | EtOH | THF | Acetone/Water (19/1) |
| None | Compound I Form I | Compound I Form | Compound I Form I |
| | | I | |
| HCl | Compound I HCl | Low crystallinity * | Compound I HCl |
| | Form I | | Form II |
| H₂SO₄ | Compound I Sulfate | Compound I | Compound I Sulfate |
| | Form I + | Sulfate Form I * | Form I * |
| | Compound I Form I | | |
| H₂SO₄ | Compound I Sulfate | Compound I | Amorphous + |
| | Form I + | Sulfate Form I + | Compound I Form I * |
| | Compound I Form I | Compound I Form | |
| | | I | |
| Maleic acid | Compound I Maleate | Compound I | Compound I Maleate |
| | Form I | Maleate Form I | Form I |
| H₃PO₄ | Compound I | Compound I | Compound I |
| | Phosphate Form I + | Phosphate Form II + | Phosphate Form II + |
| | Compound I Form I | Compound I Form | Compound I Form I * |
| | | I | |
| L-Tartaric acid | Compound I Form I | Compound I Form | Amorphous + |
| | | I | Compound I Form I * |
| Fumaric acid | Compound I Fumarate | Compound I | Compound I Fumarate |
| | Form I + | Fumarate Form I + | Form I + |
| | Compound I Form I | Compound I Form | Compound I Form I |
| | | I | |
| Citric acid | Compound I Form I + | Compound I Form | Compound I Form I + |
| | peaks | I + peaks | peaks * |
| Succinic acid | Compound I Form I | Form I + | Compound I Form I + |
| | | Compound I | Compound I |
| | | Succinate Form I | Succinate Form I |
| PTSA | Compound I Tosylate | Low crystallinity | Compound I Tosylate |
| | Form I + | | Form I * |
| | Compound I Form I * | | |
| Methanesulfonic | Sticky solid * | Compound I | Sticky solid * |
| acid | | Mesylate Form I | |

* 0.5 mL more solvent was added to dilute the thick suspensions at 16 h.

About 25 mg of Compound I Form I was suspended in selected solvents at RT, the detailed information and the results are presented in Table 32. Compound I Form I remained unchanged in selected solvent systems.

TABLE 32
Preparation of Freebase

| Solvent | | |
|---|---|---|
| (v/v, Vol.) | Crystallization Procedure | Result |
| EtOH (20 V) | Stirred for 24 h | Compound I Form I |
| THF (20 V) | Stirred for 24 h | Compound I Form I |
| Acetone/Water | Stirred for 24 h | Compound I Form I |
| (19/1, 20 V) | | |

### HCl Salt

About 25 mg of Compound I Form I was suspended in selected solvents at RT; then, 1.1 eq. of HCl was added into the suspension. The detailed information and the results are summarized in Table 33. Two crystalline HCl salts were obtained and assigned as Compound I HCl Form I and Form II, respectively. The characterization results are presented in FIG. 17A to FIG. 18B.

Compound I HCl Form I was obtained in EtOH. The sample had 4.1% weight loss before 120° C. and 6.2% weight loss from 230 to 280° C. in TGA. Two endothermic peaks at 95 and 271° C. and one exothermic peak at 165° C. (onset) were observed before decomposition by DSC, due to dehydration, melting and phase transition. No EtOH residue was detected by ¹H-NMR.

Compound I HCl Form II was obtained in acetone/water (19/1, v/v). The sample had 9.5% weight loss before 115° C. and 5.9% weight loss from 200 to 285° C. in TGA. Two endothermic peaks at 44 and 272° C. (onset) were detected by DSC, due to dehydration and melting. Negligible acetone residue was detected by ¹H-NMR.

TABLE 33
Preparation of HCl Salt

| Solvent | | |
|---|---|---|
| (v/v, Vol.) | Crystallization Procedure | Result |
| EtOH (20 V) | Stirred for 24 h | Compound I HCl |
| | | Form I |
| THF (20 V) | Stirred for 24 h | LC |
| Acetone/Water | Stirred for 24 h | Compound I HCl |
| (19/1, 20 V) | | Form II |

### Sulfate

About 25 mg of Compound I Form I was suspended in selected solvents at RT; then, 1.1 eq. or 0.55 eq. of H₂SO₄ was added into the suspension. The detailed information and the results are summarized in Table 34. Compound I Sulfate Form I with moderate crystallinity was obtained. The characterization results are presented in FIG. 19A and FIG. 19B.

Compound I Sulfate Form I was obtained in acetone/water (19/1, v/v) with 1.1 eq. H₂SO₄. The sample had 1.1% weight loss before 83° C. and 16.4% weight loss from 190 to 220° C. in TGA. Two endothermic peaks at 27 and 264° C. (onset) were detected in DSC. Negligible acetone residue was detected by ¹H-NMR.

TABLE 34
Preparation of Sulfate

| Solvent | Acid | | |
|---|---|---|---|
| (v/v, Vol.) | (eq.) | Crystallization Procedure | Result |
| EtOH (40 V) | 1.1 | After stirring for 16 h, 0.5 mL more | Compound I |
| | | EtOH was added to dilute thick system | Sulfate Form I + |
| | | and then stirred for 8 h | Compound I |
| | | | Form I |
| THF (40 V) | 1.1 | After stirring for 16 h, 0.5 mL more | Compound I |
| | | THF was added to dilute thick system | Sulfate Form I |
| | | and then stirred for 8 h | |
| Acetone/Water | 1.1 | After stirring for 16 h, 0.5 mL more | Compound I |
| (19/1, 40 V) | | acetone/water (19/1) was added to | Sulfate Form I |
| | | dilute thick system and then stirred for | |
| | | 8 h | |
| EtOH (20 V) | 0.55 | Stirred for 24 h | Compound I |
| | | | Form I + |
| | | | Compound I |
| | | | Sulfate Form I |
| THF (20 V) | 0.55 | Stirred for 24 h | Compound I |
| | | | Form I + |
| | | | Compound I |
| | | | Sulfate Form I |
| Acetone/Water | 0.55 | After stirring for 16 h, 0.5 mL more | Amorphous + |
| (19/1, 40 V) | | acetone/water (19/1) was added to | peaks |
| | | dilute thick system and then stirred for | |
| | | 8 h | |

### Maleate

About 25 mg of Compound I Form I was suspended in selected solvents at RT; then, 1.1 eq. of maleic acid was added into the suspension. The detailed information and the results are summarized in Table 35. One crystalline maleate of Form I was obtained. The characterization results are presented in FIG. 20A and FIG. 20B.

Compound I Maleate Form I was obtained in EtOH. The sample had 5.4% weight loss before 125° C. and 19.8% weight loss from 165 to 240° C. in TGA. Three endothermic peaks at 27, 208 and 284° C. (onset) were detected in DSC, due to dehydration, dissociation of salt and melting of freebase, respectively. Negligible EtOH residue was detected by ¹H-NMR, and the salt ratio was determined to be 1:1.

TABLE 35
Preparation of Maleate

| Solvent | | |
|---|---|---|
| (v/v, Vol.) | Crystallization Procedure | Result |
| EtOH (20 V) | Stirred for 24 h | Compound I |
| | | Maleate Form I |
| THF (20 V) | Stirred for 24 h | Compound I |
| | | Maleate Form I |
| Acetone/Water | Stirred for 24 h | Compound I |
| (19/1, 20 V) | | Maleate Form I |

### Phosphate

About 25 mg of Compound I Form I was suspended in selected solvents at RT; then, 1.1 eq. of H₃PO₄ was added into the suspension. The detailed information and the results are summarized in Table 36. Two crystalline phosphate salts were obtained and assigned as Compound I Phosphate Form I and II, respectively.

Compound I Phosphate Form I was obtained in EtOH and mixed with Compound I Form I. The sample had no weight loss before melting in TGA. One endothermic peak at 263° C. (onset) was detected in DSC, due to melting (TGA and DSC not shown). About 0.5% EtOH residue was detected by H-NMR.

Compound I Phosphate Form II was obtained in acetone/water (19/1, v/v) and mixed with Compound I Form I. The characterization results are presented in FIG. 21A and FIG. 21B. The sample had 2.8% weight loss before 100° C. and 1.4% weight loss from 220 to 270° C. in TGA. Three endothermic peaks at 26, 227 and 275° C. (onset) were detected in DSC, the first peak was possibly due to dehydration. About 0.4% acetone residue was detected by ¹H-NMR.

TABLE 36
Preparation of Phosphate

| Solvent | | |
|---|---|---|
| (v/v, Vol.) | Crystallization Procedure | Result |
| EtOH (20 V) | Stirred for 24 h | Compound I |
| | | Phosphate Form I + |
| | | Compound I |
| | | Form I |
| THF (20 V) | Stirred for 24 h | Compound I |
| | | Phosphate Form II + |
| | | Compound I |
| | | Form I |
| Acetone/Water | After stirring for 16 h, | Compound I |
| (19/1, 40 V) | 0.5 mL more acetone/water | Phosphate Form II + |
| | (19/1) was added to dilute | Compound I |
| | thick system and then | Form I |
| | stirred for 8 h | |

### Tartrate

About 25 mg of Compound I Form I was suspended in selected solvents at RT; then, 1.1 eq. of L-tartaric acid was added into the suspension. The detailed information and the results are summarized in Table 37.

No crystalline tartrate was obtained in three solvents.

TABLE 37
Preparation of Tartrate

| Solvent | | |
|---|---|---|
| (v/v, Vol.) | Crystallization Procedure | Result |
| EtOH (20 V) | Stirred for 24 h | Compound I Form I |
| THF (20 V) | Stirred for 24 h | Compound I Form I |
| Acetone/Water | After stirring for 16 h, 0.5 mL | Amorphous + |
| (19/1, 40 V) | more acetone/water (19/1) was | peaks |
| | added to dilute thick system | |
| | and then stirred for 8 h | |

### Fumarate

About 25 mg of Compound I Form I was suspended in selected solvents at RT; then, 1.1 eq. of fumaric acid was added into the suspension. The detailed information and the results are summarized in Table 38. One crystalline fumarate of Form I was obtained.

Compound I Fumarate Form I was obtained in acetone/water (19/1, v/v) and mixed with Compound I Form I. The sample had no weight loss before 150° C. in TGA. Two endothermic peaks at 210 and 256° C. (onset) were detected in DSC. Negligible acetone residue and 0.76 eq. acid were detected by ¹H-NMR.

TABLE 38
Preparation of Fumarate

| Solvent | | |
|---|---|---|
| (v/v, Vol.) | Crystallization Procedure | Result |
| EtOH (20 V) | Stirred for 24 h | Compound I Fumarate |
| | | Form I + Compound I |
| | | Form I |
| THF (20 V) | Stirred for 24 h | Compound I Fumarate |
| | | Form I + Compound I |
| | | Form I |
| Acetone/Water | Stirred for 24 h | Compound I Fumarate |
| (19/1, 20 V) | | Form I + Compound I |
| | | Form I |

### Citrate

About 25 mg of Compound I Form I was suspended in selected solvents at RT; then, 1.1 eq. of citric acid was added into the suspension. The detailed information and the results are summarized in Table 39. Compound I Form I with small additional peaks was obtained.

TABLE 39
Preparation of Citrate

| Solvent | | |
|---|---|---|
| (v/v, Vol.) | Crystallization Procedure | Result |
| EtOH (20 V) | Stirred for 24 h | Compound I |
| | | Form I + peaks |
| THF (20 V) | Stirred for 24 h | Compound I |
| | | Form I + peaks |
| Acetone/Water | After stirring for 16 h, 0.5 mL | Compound I |
| (19/1, 40 V) | more acetone/water (19/1) was | Form I + peaks |
| | added to dilute thick system | |
| | and then stirred for 8 h | |

### Succinate

About 25 mg of Compound I Form I was suspended in selected solvents at RT; then, 1.1 eq. of succinic acid was added into the suspension. The detailed information and the results are summarized in Table 40. A mixture of Compound I Succinate Form I and Compound I Form I was obtained in acetone/water and THF.

TABLE 40
Preparation of Maleate

| Solvent | | |
|---|---|---|
| (v/v, Vol.) | Crystallization Procedure | Result |
| EtOH (20 V) | Stirred for 24 h | Compound I Form I |
| THF (20 V) | Stirred for 24 h | Compound I Form I + |
| | | Compound I Succinate |
| | | Form I |
| Acetone/Water | Stirred for 24 h | Compound I Form I + |
| (19/1, 20 V) | | Compound I Succinate |
| | | Form I |

Compound I Succinate Form I was repeated. About 25 mg of Compound I Form I was suspended in 0.5 mL of acetone/water (19/1, v/v). Then 2.5 eq. of succinic acid was added into the suspension and the mixture was stirred at RT for 3 d. The suspension was filtered and the solids were vacuum dried at 40° C. for 14 h.

Compound I Succinate Form I was prepared successfully, but mixed with minor Compound I Form I.

The characterization results are presented in FIG. 22A to FIG. 22B.

Compound I Succinate Form I was obtained in acetone/water (19/1, v/v). The sample had 11.5% weight loss from 160 to 235° C. and 9.7% weight loss from 235 to 275° C. in TGA. Four endothermic peaks at 157, 172, 231 and 281° C. (onset) were detected in DSC. Negligible acetone residue was detected by ¹H-NMR and the salt ratio was determined to be 1:1.

### Tosylate

About 25 mg of Compound I Form I was suspended in selected solvents at RT; then, 1.1 eq. of PTSA was added into the suspension. The detailed information and the results are summarized in Table 41. One crystalline tosylate of Form I was obtained and assigned as Compound I Tosylate Form I. The characterization results are presented in FIG. 23A and FIG. 23B.

Compound I Tosylate Form I was obtained in acetone/water (19/1, v/v). The sample had 0.9% weight loss before 90° C., 2.5% weight loss from 90 to 147° C. and 0.7% weight loss from 210 to 255° C. in TGA. Three endothermic peaks at 26, 104 and 268° C. and one exothermic peak at 212° C. (onset) were detected by DSC. Negligible acetone residue was detected by ¹H-NMR, and the salt ratio was determined to be 1:1.

TABLE 41
Preparation of Tosylate

| Solvent | | |
|---|---|---|
| (v/v, Vol.) | Crystallization Procedure | Result |
| EtOH (40 V) | After stirring for 16 h, 0.5 mL more | Compound I |
| | EtOH was added to dilute thick system | Tosylate |
| | and then stirred for 8 h | Form I + |
| | | Compound I |
| | | Form I |
| THF (20 V) | Stirred for 24 h | LC |
| Acetone/Water | After stirring for 16 h, 0.5 mL more | Compound I |
| (19/1, 40 V) | acetone/water (19/1) was added to | Tosylate |
| | dilute thick system and then stirred | Form I |
| | for 8 h | |

### Mesylate

About 25 mg of Compound I Form I was suspended in selected solvents at RT; then, 1.1 eq. of methanesulfonic acid was added into the suspension. The detailed information and the results are summarized in Table 42. Compound I Mesylate Form I was obtained in THF. The characterization results are presented in FIG. 24A and FIG. 24B.

The sample had 3.0% weight loss before 115° C. in TGA. Two endothermic peaks at 27 and 284° C. (onset) were detected in DSC. About 0.8% THF was detected by ¹H-NMR, and the salt ratio was determined to be 1:1.

TABLE 42
Preparation of Mesylate

| Solvent | | |
|---|---|---|
| (v/v, Vol.) | Crystallization Procedure | Result |
| EtOH (20 V) | Stirred for 24 h | Sticky solid |
| THF (20 V) | Stirred for 24 h | Compound I |
| | | Mesylate |
| | | Form I |
| Acetone/Water | After stirring for 16 h, 0.5 mL more | Sticky solid |
| (19/1, 40 V) | Acetone/water (19/1) was added to dilute | |
| | thick system and then stirred for 8 h | |

### Scale-Up and Full Characterization

Compound I Phosphate Form I and Compound I Fumarate Form I were scaled up at 350-mg scale for full characterization and DVS analysis. Detailed experimental procedures are described in Table 43.

Compound I Phosphate Form I was obtained with a yield of about 94% and characterized by XRPD, DSC, TGA and ¹H-NMR. Compound I Phosphate Form I was highly crystalline and composed of fine crystals (FIG. 15D). TGA showed no weight loss before 200° C., and one single endothermic peak at 262° C. (onset) was observed in DSC, due to melting (FIG. 15E). About 0.7% acetone residue was detected by ¹H-NMR. The salt ratio of base to acid was determined to be 1:2 by IC (28.7% PO₄³⁻). Therefore, Compound I Phosphate Form I is an anhydrous di-salt.

Compound I Fumarate Form I was obtained with a yield of about 84% and characterized by XRPD, DSC, TGA and ¹H-NMR. Compound I Fumarate Form I was highly crystalline and composed of fine crystals with slight aggregation (FIG. 16D). TGA showed no weight loss before 150° C., 3.0% weight loss from 200 to 260° C., and two endothermic peaks at 213 and 260° C. (onset) were observed in DSC (FIG. 16E). About 0.4% acetone residue and 1.0 eq. fumaric acid were detected by ¹H-NMR. Thermal treatment was performed to study the first small endothermic peak. After heating to 230° C., the crystal form remained unchanged and the small endothermic peak disappeared in DSC (FIG. 16G). After further heating to 250° C., a mixture of Compound I Fumarate Form I and Compound I Form I was obtained (FIG. 16G).

TABLE 43
Re-preparation of Compound I Phosphate
Form I and Compound I Fumarate Form I

| | |
|---|---|
| Crystallization Procedure | Result |

| | | |
|---|---|---|
| 1) | 25 mg of Compound I Form I was weighed into a 1.5 mL | Compound I |
| | vial. 2.0 eq. of H₃PO₄was added, and 0.5 mL | Phosphate |
| | MeOH/DCM (4/1, v/v) was added. Trace seed, which was | Form I |
| | made according to methods described herein, was added, | |
| | and the mixture was stirred at room temperature for about | |
| | 24 h. The suspensions were filtered, and the solids were | |
| | vacuum dried at about 40° C. for about 14 h to produce | |
| | seeds used in this procedure. | |
| 2) | 350.0 mg of Compound I Form I and 265 mg (3.0 eq.) of | |
| | H₃PO₄ (85% wt.) were weighed into an 8-mL glass vial. | |
| 3) | 7 mL acetone/water (19/1, v/v) was added, the mixture | |
| | was stirred at about 50° C. Then 5 mg of Compound I | |
| | Phosphate Form I seed, which was made according to | |
| | methods described herein, was added and stirred at about | |
| | 50° C. for about 2 d. | |
| 4) | The suspension was filtered, and the filter cake was | |
| | vacuum dried at about 40° C. for about 6 h. | |
| 1) | 350.0 mg of Compound I Form I and 180 mg of fumaric | Compound I |
| | acid (2.0 eq.) were weighed into an 8-mL glass vial. | Fumarate |
| 2) | 7 mL acetone/water (19/1, v/v) was added, and the | Form I |
| | mixture was stirred at about 50° C. Then 5 mg of | |
| | Compound I Fumarate Form I seed, which was made | |
| | according to methods described herein, was added and | |
| | stirred at about 50° C. for about 2 d. | |
| 3) | The suspension was filtered, and the filter cake was | |
| | vacuum dried at about 40° C. for about 14 h. | |

### Example 4: Phosphate Polymorph Screen

### Analysis Methods

### XRPD

XRPD diffractograms were collected with an X-ray diffractometer. The sample was prepared on a zero-background silicon wafer by gently pressing onto the flat surface. The parameters of XRPD diffraction are given in Table 44.

TABLE 44
Parameters for XRPD Testing

| Instrument | Bruker, D8 Advance | |
|---|---|---|
| Radiation | Cu Ka (l = 1.5418 Å) | |
| Detector | LynxEye | |
| Scan angle | 3-40° | (2q) |
| Scan step | 0.013° | (2q) |
| Scan speed | 0.1 | s/step |
| Tube voltage/current | 40 kV/40 mA | |
| Divergence slit | 0.6 | mm |

| | |
|---|---|
| Rotation | On |
| Sample holder | Zero-background sample pan |

### TGA

TGA analysis was performed using a TA Instrument. About 1-5 mg of a sample was loaded onto a pre-tared aluminum pan and heated with the parameters in Table 45. The data was analyzed using TRIOS.

TABLE 45
Parameters for TGA Testing

| Instrument | TA TGA 5500 | |
|---|---|---|
| Sample pan | Aluminum, open | |
| Temperature range | RT-300° | C. |
| Heating rate | 10° | C./min |

| | |
|---|---|
| Purge gas | N₂ |
| Flow rate | Balance chamber: 40 mL/min |
| | Sample chamber: 60 mL/min |

### DSC

DSC analysis was performed with a TA Instrument. About 1-3 mg of a sample was placed into an aluminum pan with pin-hole and heated with the parameters in Table 46. The data was analyzed using TRIOS.

TABLE 46
Parameters for DSC Testing

| Instrument | TA DSC 2500 | |
|---|---|---|
| Sample pan | Aluminum, pin-holed | |
| Temperature range | 25-300° | C. |
| Heating rate | 10° | C./min |
| Purge gas | N₂ | |
| Flow rate | 50 | mL/min |

### ¹H-NMR

¹H-NMR spectra were collected on a Bruker 400 MHz instrument. Unless specified, samples were prepared in DMSO-d6 solvent and measured with the parameters in Table 47. The data was analyzed using MestReNova.

TABLE 47
Parameters for ¹H-NMR Analysis

| Instrument | Bruker | |
|---|---|---|
| Frequency | 400 | MHz |
| Scan times | 4 | |
| Temperature | 295 | K |
| Relaxation delay | 1 | s |

### HPLC

HPLC analysis was performed with an Agilent HPLC 1260 series instrument. HPLC method for solubility and stability testing is presented in Table 48.

TABLE 48
HPLC Method for Purity Testing

| | |
|---|---|
| Instrument | Agilent 1260 HPLC series |
| Column | Ascentis Express C18, 4.6 × 100 mm, 2.7 μm |
| Mobile Phase | A: 0.05% TFA in water; B: 0.05% TFA in ACN |
| Gradient (T/B %) | 0.0/5%, 7/20%, 8/20%, 13/35%, |
| | 16/95%, 17/95%, 17.1/5% |
| Column Temperature | 30° C. |
| Detector | DAD; 220 nm |
| Flow Rate | 1 mL/min |
| Injection Volume | 2 or 3 mL |
| Run Time | 17.10 min |
| Post Time | 3 min |
| Diluent | MeOH/water (v/v, 9:1) |

### Summary

In this study, the objective was to conduct a polymorph screen for the phosphate of Compound I. The free base of Compound I, which can be prepared according to methods described herein, was used to prepare Compound I Phosphate Form I for polymorph screen.

Polymorph screening was carried out using solvents and various crystallization methods, including slurry conversion, cooling, evaporation, anti-solvent precipitation, cyclic heating-cooling and reactive crystallization. Seven crystal forms were found, including four anhydrates of Compound I Phosphate Forms I, II, VI and VII, the other forms were either solvate or metastable form. Compound I Phosphate Form I and Compound I Phosphate Form II are anhydrates, while Compound I Phosphate Form VI and Compound I Phosphate Form VII were identified after heating to high temperature of 200° C.; chemical changes were observed for Compound I Phosphate Form VII. Compound I Phosphate Forms III-VI were obtained in high polar solvents of water and NMP.

Slurry competition experiments were performed between the two anhydrates of Compound I Phosphate Forms I and II in EtOH and acetone/water (19/1, v/v) at RT and 50° C. After 3 days, Compound I Phosphate Form II converted to Compound I Phosphate Form I at all conditions, suggesting that Compound I Phosphate Form I is more thermodynamically stable than Compound I Phosphate Form II at RT-50° C. Compound I Phosphate Form I is characterized by a differential scanning calorimetry (DSC) curve comprising an endotherm at about 262° C. (onset temperature) as shown in FIG. 15E, thermogravimetric analysis (TGA) showing no weight loss up to about 200° C. as shown in FIG. 15E or FIG. 15F, and a dynamic vapor sorption (DVS) curve showing about 0.70% water uptake at 80% relative humidity (RH) or about 0.81% water uptake at 90% RH as shown in FIG. 15G.

Overall, Compound I Phosphate Form I, a di-phosphate, is a stable anhydrate with acceptable solid-state properties, good physicochemical stability, and improved aqueous solubility.

### Characterization and Definition of Crystal Forms

A total of seven crystal forms, Compound I Phosphate Forms I-VII, were identified. Among them, Compound I Phosphate Forms I, II, VI and VII are anhydrates. Compound I Phosphate Forms III-VI were obtained in high polar solvents.

### Compound I Phosphate Form I

About 2.2 g of Compound I Form I, which can be prepared according to methods described herein was added into 73.3 mL acetone/water (19/1, v/v), then 3.0 eq. H₃PO₄ was added. Compound I Phosphate Form I seed, which was made according to methods described herein, was added and the mixture was stirred at about 50° C. for about 2 days. The solid was collected by filtration and dried at about 40° C. under vacuum for about 3 h. Compound I Phosphate Form I was characterized by XRPD, TGA, DSC, ¹H-NMR and IC. Compound I Phosphate Form I is characterized by a differential scanning calorimetry (DSC) curve comprising an endotherm at about 251° C. (onset temperature) as shown in FIG. 15F.

The sample showed small rod crystals with slight aggregation. XRPD showed it was Compound I Phosphate Form I with high crystallinity. Thermal analysis showed negligible weight loss before melting in TGA, and one endothermic peak at 251° C. (onset) in DSC, due to melting. 0.3% acetone was detected by ¹H-NMR. The salt ratio of acid to base was determined to 2:1 by IC (29.9% PO₄³⁻.

### Compound I Phosphate Form II

Compound I Phosphate Form II was obtained by reactive crystallization starting from Compound I Form I in EtOH at about 50° C., which was characterized by XRPD, TGA, DSC and ¹H-NMR. XRPD (FIG. 21A) showed Compound I Phosphate Form II was fine crystals with aggregation and high crystallinity. Thermal analysis (FIG. 21C) showed 0.1% weight loss at 165-180° C. and 2.4% weight loss at 210-265° C. in TGA, and three endothermic peaks at 170, 223 and 275° C. (onset) in DSC. ¹H-NMR showed 0.4% residual EtOH. Therefore, Compound I Phosphate Form II is an anhydrate.

After heating to 200° C., Compound I Phosphate Form II remained unchanged and after heating to 263° C., Compound I Phosphate Form II converted to Compound I Phosphate Form VII, which showed similar XRPD with Compound I Phosphate Form II with slight peak shift.

### Compound I Phosphate Form III

Compound I Phosphate Form III was obtained by slurry of Compound I Phosphate Form I in NMP at RT, after air drying at RT for 2 days, Compound I Phosphate Form III mostly converted to Compound I Phosphate Form V (FIG. 25A).

### Compound I Phosphate Form IV

Compound I Phosphate Form IV was obtained by slurry of Compound I Phosphate Form I in water at RT for 7 days. After drying at 40° C. under vacuum overnight, the crystallinity of Compound I Phosphate Form IV decreased significantly (FIG. 26A).

### Compound I Phosphate Form V

Compound I Phosphate Form V was obtained by slurry of Compound I Phosphate Form I in NMP at 50° C., which was characterized by XRPD, TGA, DSC and ¹H-NMR. The characterization data are shown in FIG. 27A and FIG. 27B.

XRPD (FIG. 27A) showed Compound I Phosphate Form V was small rod crystals with slight aggregation and high crystallinity. Thermal analysis (FIG. 27B) showed 2.8% weight loss at 40-100, 12.1% weight loss at 100-180° C., and 11.3% weight loss at 180-210° C. in TGA, and multiple thermal events in DSC.

¹H-NMR showed 24.8% NMP. Therefore, Compound I Phosphate Form V is a solvate of NMP. After heating to 90 and 155° C., Compound I Phosphate Form V remained unchanged. After heating to 210° C., Compound I Phosphate Form V converted to Compound I Phosphate Form VI.

### Compound I Phosphate Form VI

Compound I Phosphate Form VI was obtained by heating Compound I Phosphate Form V to 210° C., which was characterized by XRPD, TGA, DSC and ¹H-NMR. The characterization data are shown in FIG. 28A and FIG. 28B.

XRPD (FIG. 28A) showed Compound I Phosphate Form VI was fine crystals with aggregation and high crystallinity. Thermal analysis (FIG. 28B) showed negligible weight loss before melting in TGA, and one endothermic peak at 246° C. (onset) in DSC, due to melting. ¹H-NMR showed negligible organic solvent. Therefore, Compound I Phosphate Form VI is an anhydrate.

In order to study the relationship between Compound I Phosphate Form VI and Compound I Phosphate Form I, slurry experiments were conducted. About 5 mg Compound I Phosphate Form VI was suspended into 0.2-0.3 mL EtOH at RT or 50° C. for 1 h; then, a small amount of Compound I Phosphate Form I seed, which was made according to methods described herein, was added. The suspension was stirred at RT or 50° C. for up to 5 days. Samples were collected by filtration and characterized by XRPD. The results are summarized in Table 49.

No form conversion was found at RT, but Compound I Phosphate Form VI converted to Compound I Phosphate Form I with 1 eq. H₃PO₄ at RT for 1 day. Compound I Phosphate Form VI converted to Compound I Phosphate Form I and Compound I Form III at 50° C.

TABLE 49
Results of Slurry Study of Compound I Phosphate Form VI

| Solvent | Temp | XRPD Result | | | |
|---|---|---|---|---|---|
| (V) | (° C.) | 1 h | 1 d | 2 d | 5 d |
| EtOH (60 V) | RT | Compound I | Compound I | Compound I | Compound I |
| | | Phosphate | Phosphate Form | Phosphate Form | Phosphate |
| | | Form VI | VI + I | VI + I | Form VI + I |
| | | | (trace) | (trace) | (trace) |
| | 50° C. | Compound I | Compound I | Compound I | / |
| | | Phosphate | Phosphate Form | Phosphate Form I + | |
| | | Form VI | VI + I | VI (trace) + | |
| | | | (trace) | Compound I | |
| | | | | Phosphate | |
| | | | | Form III | |
| EtOH (40 V) | RT | / | Compound I | / | / |
| 1.0 eq. H₃PO₄ | | | Phosphate | | |
| | | | Form I | | |

### Compound I Phosphate Form VII

Compound I Phosphate Form VII was obtained by heating Compound I Phosphate Form II to 263° C., which was characterized by XRPD, TGA and DSC. The characterization data are shown in FIG. 29A and FIG. 29B.

XRPD (FIG. 29A) showed Compound I Phosphate Form VII was fine crystals with slight aggregation and high crystallinity. Thermal analysis (FIG. 29B) showed 0.2% weight loss before 115° C. in TGA, and one endothermic peak at 270° C. (onset) in DSC, due to melting. ¹H-NMR showed negligible organic solvent. Therefore, Compound I Phosphate Form VII is an anhydrate. [0509]¹H-NMR showed Compound I Phosphate Form VII had a change of chemical shift, compared to Compound I Form I and Compound I Phosphate Form I HPLC showed one 3.1% impurity. Slurry competition experiments were conducted. About 4 mg each of Compound I Phosphate Form VII and Compound I Phosphate Form I were added into 0.4 mL EtOH at RT, 35° C. or 50° C. which was pre-saturated by Compound I Phosphate Form I. The suspension was stirred at RT, 35° C. or 50° C. for 2 d. Samples were collected by filtration and characterized by XRPD. The results are presented in Table 50 No obvious conversion was observed.

TABLE 50
Results of Slurry Competition Study of
Compound I Phosphate Forms I and VII

| | |
|---|---|
| Temp | XRPD Result |

| Solvent (V) | (° C.) | 1 d | 2 d |
|---|---|---|---|
| EtOH | RT | Compound I | Compound I |
| (50 V) | | Phosphate | Phosphate |
| | | Forms I + VII | Forms I + VII |
| | 35° C. | Compound I | Compound I |
| | | Phosphate | Phosphate |
| | | Forms I + VII | Forms I + VII |
| | 50° C. | Compound I | Compound I |
| | | Phosphate | Phosphate |
| | | Forms I + VII | Forms I + VII |

### Stability Relationship Study

Slurry competition experiments were conducted between Compound I Phosphate Forms I and II. About 5 mg each of Compound I Phosphate Form I and Compound I Phosphate Form II were added into 1 mL solvent at RT and 50° C. which was pre-saturated by Compound I Phosphate Form I. The sample was analyzed by XRPD at Day 3. The results are presented in Table 51. Compound I Phosphate Form I was obtained at all conditions, indicating Compound I Phosphate Form I is thermodynamically more stable at RT-50° C.

TABLE 51
Results of Competition Slurry of Compound
I Phosphate Forms I and II

| Solvent (v/v) | Temperature | XRPD - 3 d |
|---|---|---|
| EtOH | RT | Compound I |
| | | Phosphate |
| | | Form I |
| EtOH | 50° C. | Compound I |
| | | Phosphate |
| | | Form I |
| Acetone/Water | RT | Compound I |
| (19/1) | | Phosphate |
| | | Form I |
| Acetone/Water | 50° C. | Compound I |
| (19/1) | | Phosphate |
| | | Form I |

### pH Solubility Profile of Compound I Phosphate Form I

Different pH buffers (pH 1.0, 3.0, 5.0, 6.8 and 9.0) were prepared by the method described in Table 52. Then, 10 mg of Compound I Phosphate Form I was added into 2 mL of different buffers (pH=1.0, 3.0, 5.0, 6.8 and 9.0) to make suspensions. The suspensions were kept shaking with a speed of 1000 rpm at 25° C. for 4 and 24 hours. At each point, the suspensions were centrifuged, and the supernatant was inspected by HPLC/pH, and the wet cakes were analyzed by XRPD. Duplicate samples were prepared.

All the results are summarized in Table 53. Form conversion was observed at all tested pH buffers, and the solubility was overall pH dependent. For the two new patterns (“NP1” and “NP2”), NP1 was of low crystallinity and not further characterized, while NP2 was characterized by TGA and DSC. Thermal analysis showed 5.6% weight loss at 45-115° C. and 0.5% weight loss at 160-215° C. in TGA, and three endothermic peaks at 57, 122 and 285° C. (onset) in DSC. After heating to 240° C., NP2 converted into Compound I Form I.

TABLE 52
Preparation of Different pH Buffers

| | |
|---|---|
| pH Buffer | Experimental Procedure |

| | | |
|---|---|---|
| pH 1.0 | 0.1N HCl | / |
| pH 3.0 | 25 mM citrate, | Anhydrate citric acid (0.480 g), NaOH (0.2M, |
| | I = 0.1M | 5.856 mL) and NaCl (0.526 g) was dissolved |
| | | in water, the total volume of water is 100 mL. |
| pH 5.0 | 25 mM citrate, | Anhydrate citric acid (0.480 g), NaOH (0.2M, |
| | I = 0.1M | 23.818 mL) and NaCl (0.178 g) was dissolved |
| | | in water, the total volume of water is 100 mL. |
| pH 6.8 | 25 mM Phosphate, | Monohydrate NaH₂PO₄ (0.345 g), NaOH |
| | I = 0.1M | (0.2M, 5.904 mL) and NaCl (0.335 g) was |
| | | dissolved in water, the total volume of |
| | | water is 100 mL. |
| pH 9.0 | 25 mM DEA, | Diethanolamine (0.2629 g), HCl (0.1M, 12.375 |
| | I = 0.1M | mL) and NaCl (0.580 g) was dissolved in |
| | | water, the total volume of water is 100 mL. |

TABLE 53
pH Solubility Results

| | Initial | Solubility (mg/mL) | | pH | | XRPD | |
|---|---|---|---|---|---|---|---|
| pH Buffer | pH | 4 h | 24 h | 4 h | 24 h | 0.5 h | 24 h |
| pH 1.0 | 1.00 | 3.61/2.09 | 1.81 | 0.93 | 0.98 | NP1 | NP1 |
| | | | | | | (little solid) | |
| pH 3.0 | 2.95 | >3.73 | 2.67 | 2.66 | 2.67 | Clear | NP1 |
| pH 5.0 | 5.03 | 0.27 | 0.20 | 4.18 | 4.21 | NP2 | NP2 |
| pH 6.8 | 6.79 | 1.72 | 1.55 | 3.35 | 3.33 | Gel | NP2 |
| pH 9.0 | 9.05 | 1.34 | 1.32 | 3.50 | 3.55 | NP2 | NP2 |

NP: new pattern

### Mechanical Stability of Compound I Phosphate Form I

An appropriate amount of Compound I Phosphate Form I was manually ground by pestle and mortar for about 2 minutes and 5 minutes, respectively. The ground samples were analyzed by XRPD. The crystal form of Compound I Phosphate Form I remained unchanged with little crystallinity decrease after grinding, indicating good mechanical stability.

### Polymorph Screening

Solvents used for the polymorph screening are given in Table 54.

TABLE 54
List of Solvents

| # | Solvent |
|---|---|
| 1 | MeOH |
| 2 | EtOH |
| 3 | DMF |
| 4 | IPA |
| 5 | Acetone |
| 6 | MEK |
| 7 | ACN |
| 8 | EtOAc |
| 9 | IPAc |
| 10 | THF |
| 11 | DCM |
| 12 | Water |
| 13 | MTBE |
| 14 | DMSO |
| 15 | NMP |
| 16 | MIBK |
| 17 | Toluene |
| 18 | DMF |

Polymorph screening experiments are summarized in Table 55. Six crystalline forms of Compound I Phosphate Forms I-VI were found, Compound I Phosphate Form VII was obtained via thermal treatment of Compound I Phosphate Form II.

TABLE 55
Summary of Polymorph Screening

| No. | Method | Crystalline Form |
|---|---|---|
| 1 | Reactive crystallization | Compound I Phosphate Forms I, II |
| 1 | Slurry conversion | Compound I Phosphate Forms I, |
| | | III, IV, V, VI |
| 2 | Anti-solvent precipitation | Compound I Phosphate Forms I, II, V |
| 3 | Evaporation | Compound I Phosphate Forms I, IV |
| | crystallization | |
| 4 | Cooling crystallization | Compound I Phosphate Forms I, IV |
| 5 | Cyclic heating-cooling | Compound I Phosphate Forms I, VI |

| | |
|---|---|
| Total | Compound I Phosphate Forms I-VI |

### Slurry Conversion

An appropriate amount of Compound I Phosphate Form I was added into different solvents to make suspensions which were kept stirring at RT and 50° C. for 3 and 7 days and at 80° C. for 3 days. Solid samples were collected by filtration and analyzed by XRPD.

Most samples obtained was Compound I Phosphate Form I in slurry conversion. Compound I Phosphate Form III and Compound I Phosphate Form V were obtained in NMP at RT or 50° C., respectively. Compound I Phosphate Form IV was obtained in water at RT. A mixture of Compound I Phosphate Form I and Compound I Phosphate VI was obtained in DMF at RT and 50 IC. The results are summarized in Table 56-Table 58.

TABLE 56
Results of Slurry at RT

| | XRPD Result | |
|---|---|---|
| Solvent (v/v, Vol.) | 3 Days | 1 Week |
| MeOH (50 V) | Compound I | Compound I |
| | Phosphate Form I | Phosphate Form I |
| EtOH (50 V) | Compound I | Compound I |
| | Phosphate Form I | Phosphate Form I |
| Acetone (50 V) | Compound I | Compound I |
| | Phosphate Form I | Phosphate Form I |
| EtOAc (50 V) | Compound I | Compound I |
| | Phosphate Form I | Phosphate Form I |
| THF (50 V) | Compound I | Compound I |
| | Phosphate Form I | Phosphate Form I |
| NMP (50 V) | Compound I | Compound I |
| | Phosphate Form III | Phosphate Form III |
| Water (25 V) | Compound I | Compound I |
| | Phosphate Form I + IV | Phosphate Form IV |
| DMSO (25 V) | Compound I Form V | Compound I Form V |
| DMF (50 V) | Compound I | Compound I |
| | Phosphate Form I | Phosphate Form I + VI |
| ACN (50 V) | Compound I | Compound I |
| | Phosphate Form I | Phosphate Form I |
| THF/Water | Compound I | Compound I |
| (9/1, 50 V) | Phosphate Form I | Phosphate Form I |
| ACN/Water | Compound I | Compound I |
| (6/1, 50 V) | Phosphate Form I | Phosphate Form I |
| Acetone/Water | Compound I | Compound I |
| (9/1, 50 V) | Phosphate Form I | Phosphate Form I |
| MeOH/Water | Compound I | Compound I |
| (9/1, 50 V) | Phosphate Form I | Phosphate Form I |
| Acetone/MeOH | Compound I | Compound I |
| (4/1, 50 V) | Phosphate Form I | Phosphate Form I |

TABLE 57
Results of Slurry at 50° C.

| | XRPD Result | |
|---|---|---|
| Solvent (v/v, Vol.) | 3 Days | 1 Week |
| MeOH (50 V) | Compound I | Compound I |
| | Phosphate Form I | Phosphate Form I |
| EtOH (50 V) | Compound I | Compound I |
| | Phosphate Form I | Phosphate Form I |
| Acetone (50 V) | Compound I | Compound I |
| | Phosphate Form I | Phosphate Form I |
| EtOAc (50 V) | Compound I | Compound I |
| | Phosphate Form I | Phosphate Form I |
| THF (50 V) | Compound I | Compound I |
| | Phosphate Form I | Phosphate Form I |
| NMP (50 V) | Compound I | Compound I |
| | Phosphate Form V | Phosphate Form V |
| Water (25 V) | Compound I | Compound I |
| | Phosphate Form I | Phosphate Form I |
| DMSO (25 V) | Compound I Form V | Compound I Form V |
| DMF (50 V) | Compound I | Compound I |
| | Phosphate Form I | Phosphate Form I + VI |
| ACN (50 V) | Compound I | Compound I |
| | Phosphate Form I | Phosphate Form I |
| THF/Water | Compound I | Compound I |
| (9/1, 50 V) | Phosphate Form I | Phosphate Form I |
| ACN/Water | Compound I | Compound I |
| (6/1, 50 V) | Phosphate Form I | Phosphate Form I |
| Acetone/Water | Compound I | Compound I |
| (9/1, 50 V) | Phosphate Form I | Phosphate Form I |
| MeOH/Water | Compound I | Compound I |
| (9/1, 50 V) | Phosphate Form I | Phosphate Form I |
| Acetone/MeOH | Compound I | Compound I |
| (4/1, 50 V) | Phosphate Form I | Phosphate Form I |

TABLE 58
Results of Slurry at 80° C.

| Solvent (v/v, Vol.) | Slurry for 3 Days |
|---|---|
| ACN (50 V) | Compound I Phosphate Form I |
| NMP (15 V) | Compound I Phosphate Form V + I (minor) |
| DMSO (15 V) | Compound I Form I |
| IPA (50 V) | Compound I Phosphate Form I |
| Water (15 V) | Compound I Phosphate Form I |
| DMF (25 V) | Compound I Phosphate Form I + |
| | Compound I Form I + peaks |
| ACN/Water | Compound I Phosphate Form I |
| (6/1, 50 V) | |
| NMP/MIBK | Compound I Phosphate Form I |
| (1/10, 25 V) | |
| Butanol/anisole | Compound I Phosphate Form I |
| (1/1, 50 V) | |

### Cooling Crystallization

Cooling crystallization was performed in six selected solvents. About 15 mg of Compound I Phosphate Form I was weighed into a glass vial, and then a selected solvent was added to make a suspension with stirring at elevated temperature for about 20 m. Then, the suspension was filtered to obtain saturated drug solution at elevated temperature. The filtrate was placed at RT for about 8 h first; if no solid occurred, the filtrate was placed at 4° C. refrigerator for cooling crystallization. Any solid obtained was characterized by XRPD.

The results are summarized in Table 59. Clear solutions remained after kept at RT for about 8 h. After cooling to 5° C., Compound I Phosphate Form I was obtained in MeOH/water (9/1) and DMSO/ACN (1/2), and Compound I Phosphate Form IV+1 pk was obtained in THF/water (9/1).

TABLE 59
Results of Cooling Crystallization

XRPD Result

| Solvent (v/v, Vol.) | RT | 5° C. |
|---|---|---|
| MeOH (67 V) | Clear | Clear |
| THF/Water | Clear | Compound I |
| (9/1, 67 V) | | Phosphate Form IV + |
| | | 11.6° pk |
| MeOH/Water | Clear | Compound I |
| (9/1, 67 V) | | Phosphate Form I |
| ACN/Water | Clear | Clear |
| (6/1, 67 V) | | |
| Acetone/Water | Clear | Clear |
| (6/1, 67 V) | | |
| DMSO/ACN | Clear | Compound I |
| (1/2, 67 V) | | Phosphate Form I |

### Evaporation Crystallization

Evaporation crystallization was performed in five selected solvents according to the solubility data. Clear solutions or suspensions of Compound I Phosphate Form I were prepared at RT or 50° C.; after filtration, the filtrate was covered with pin-hole film and placed at ambient condition for slow evaporation. The results are summarized in Table 60. Compound I Phosphate Form I and Compound I Phosphate Form IV were obtained by evaporation, as well as two extra peaks at 3.4° and 10.3° (20).

TABLE 60
Results of Evaporation Crystallization

| Solvent (v/v, Vol.) | XRPD Result |
|---|---|
| MeOH (200 V) | Compound I Phosphate Form I + IV (minor) |
| MeOH/Water | Compound I Phosphate Form I + IV (minor) |
| (9/1, 200 V) | |
| Acetone/Water | Compound I Phosphate Form IV + |
| (6/1, 200 V) | extra peaks (3.4°, 10.3°) |
| ACN/water | Compound I Phosphate Form I |
| (6/1, 200 V) | |
| THF/Water | Compound I Phosphate Form I + |
| (9/1, 200 V) | extra peaks (3.4°, 10.3°) |

### Anti-Solvent Precipitation

Anti-solvent precipitation was performed according to solubility data. Appropriate amount of Compound I Phosphate Form I was weighed into 8 mL glass vials and then a selected solvent was added to make a nearly saturated solution. After filtration, anti-solvent was added into the filtrate gradually until solids precipitated out or 5 mL anti-solvent was reached at RT. If precipitation occurred, solids were isolated by filtration and characterized accordingly.

The results are summarized in Table 61 Compound I Phosphate Form I was obtained in DMSO/IPAc, DMSO/toluene, water/ACN and water/acetone; Compound I Phosphate Form II was obtained in MeOH/MTBE; Compound I Phosphate Form V was obtained in NMP/IPAc and NMP/MTBE.

TABLE 61
Results of Anti-solvent Precipitation

| Solvent | Anti- | | Vₛₒₗᵥₑₙₜ/ | XRPD Result | |
|---|---|---|---|---|---|
| (V) | solvent | Observation | Vₐₙₜᵢ | 1 h | 1 d |
| DMSO | IPAc | Solid occurred | 1/8 | Compound I | Compound I |
| (50 V) | | immediately | | Phosphate | Phosphate |
| | | | | Form I | Form I |
| | Toluene | Solid occurred | 1/9 | Clear | Compound I |
| | | after slurry | | | Phosphate |
| | | for 1 d | | | Form I |
| NMP | IPAc | Solid occurred | 1/2 | Compound I | Compound I |
| (50 V) | | immediately | | Phosphate | Phosphate |
| | | | | Form V | Form V |
| | MTBE | Solid occurred | 1/3 | Compound I | Compound I |
| | | immediately | | Phosphate | Phosphate |
| | | | | Form V | Form V |
| MeOH | MTBE | Solid occurred | 1/5 | Compound I | Compound I |
| (100 V) | | after slurry | | Phosphate | Phosphate |
| | | for 10 min | | Form II | Form II |
| Water | ACN | Solid occurred | 1/5 | Compound I | Compound I |
| (50 V) | | immediately | | Phosphate | Phosphate |
| | | | | Form I | Form I |
| | Acetone | Solid occurred | 1/9 | Compound I | Compound I |
| | | after slurry | | Phosphate | Phosphate |
| | | for 30 min | | Form I | Form I |

Vₛₒₗᵥₑₙₜ/Vₐₙₜᵢ is the volume ratio of solvent to anti-solvent

### Cyclic Heating-Cooling

Cyclic heating-cooling experiments were carried out in Crystal 16 Parallel Crystallizer. About 10 mg of Compound I Phosphate Form I was added into 1 m solvents, and a heating-cooling program was performed:
1. Equilibrated first at 20° C., and heated to 80° C. at 0.2° C./min
2. Held at 80° C. for 1 h
3. Cooled to 20° C. at 0.2° C./min, and held for 1 h
4. Repeated the above procedures once

Turbidimeter from Crystal 16 recorded if the solids were completely dissolved during heating. The results are presented in Table 62 Clear solution was obtained in water and no solid precipitated during cooling. Compound I Phosphate Form I was obtained in n-butanol/anisole (1/1), EtOH and ACN/water (19/1). Compound I Phosphate Form VI was obtained in DMSO/MIBK (1/3).

TABLE 62
Results of Cyclic Heating-cooling

| | Hot | XRPD |
|---|---|---|
| Solvent (v/v, Vol.) | Dissolution | Result |
| n-Butanol/Anisole | No | Compound I |
| (1/1, 100 V) | | Phosphate |
| | | Form I |
| Water (100 V) | Clear | N/A |
| EtOH (100 V) | No | Compound I |
| | | Phosphate |
| | | Form I + one |
| | | small pk |
| ACN/Water | No | Compound I |
| (19/1, 100 V) | | Phosphate |
| | | Form I |
| DMSO/MIBK | No | Compound I |
| (1/3, 100 V) | | Phosphate |
| | | Form I + VI |

### Reactive Crystallization

Reactive crystallization was performed at RT and 50° C. in different solvents. About 30 mg of Compound I Form I was dispersed in 1 mL or 0.5 mL selected solvent. Then 2.5 eq. of H₃PO₄ was added and stirred at RT or 50° C. The solid was collected by filtration and characterized by XRPD. The results are presented in Table 63 Compound I Phosphate Form I was obtained in most solvents. Compound I Phosphate Form II was obtained in EtOH at RT/50° C. and acetone/water (19/1) at RT.

TABLE 63
Results of Reactive Crystallization

| Solvent (v/v, Vol.) | Temp (° C.) | Result (3 d) |
|---|---|---|
| MeOH (33 V) | RT | Compound I |
| | | Phosphate |
| | | Form I |
| THF (33 V) | RT | Compound I |
| | | Phosphate |
| | | Form I |
| MeOH/DCM | RT | Compound I |
| (1/4, 17 V) | | Phosphate |
| | | Form I |
| Acetone/Water | RT | Compound I |
| (19/1, 33 V) | | Phosphate |
| | | Form II |
| EtOH (33 V) | RT | Compound I |
| | | Phosphate |
| | | Form II |
| MeOH/Water | RT | Compound I |
| (19/1, 33 V) | | Phosphate |
| | | Form I |
| MeOH (33 V) | 50° C. | Compound I |
| | | Phosphate |
| | | Form I |
| THF (33 V) | 50° C. | Compound I |
| | | Phosphate |
| | | Form I |
| Acetone/Water | 50° C. | Compound I |
| (19/1, 33 V) | | Phosphate |
| | | Form I |
| EtOH (33 V) | 50° C. | Compound I |
| | | Phosphate |
| | | Form II |
| MeOH/Water | 50° C. | Compound I |
| (19/1, 33 V) | | Phosphate |
| | | Form I |

### Example 5: Single Crystal Structure Determination

### Analysis Method

### XRPD

XRPD diffractograms were collected with an X-ray diffractometer. The sample was prepared on a zero-background silicon wafer by gently pressing onto the flat surface. The parameters of XRPD diffraction are given in Table 64.

TABLE 64
Parameters for XRPD Testing

| Instrument | Bruker, D8 Advance | |
|---|---|---|
| Radiation | Cu Ka (l = 1.5418 Å) | |
| Detector | LynxEye | |
| Scan angle | 3-40° | (2q) |
| Scan step | 0.013° | (2q) |
| Scan speed | 0.1 | s/step |
| Tube voltage/current | 40 kV/40 mA | |
| Divergence slit | 0.6 | mm |

| | |
|---|---|
| Rotation | On |
| Sample holder | Zero-background |
| | sample pan |

### Summary

The objective of this study was to determine the molecular structure of Compound I Phosphate Form I by single crystal X-ray diffraction. Plate-shaped single crystals were obtained by slow evaporation in MeOH at RT, which were used for single crystal X-ray diffraction.

The crystal data were collected on a Rigaku XtaLAB Synergy-DW diffractometer and analyzed using SHELXT program. The single crystal structure of Compound I Phosphate Form I is monoclinic in P2₁/c space group with formula of C₂₃H₂₉F₂N₅O₁₁P₂. There are one Compound I cation, one phosphate anion, and one phosphoric acid molecule in each asymmetric unit, and the unit cell contains four asymmetric units. Compound I Phosphate Form I is a phosphoric acid co-crystal of phosphate. The stoichiometric ratio of Compound I to phosphoric acid is determined as 1:2. The calculated XRPD pattern from single crystal structure matches well with the experimental XRPD pattern.

### Single Crystal Growth and Data Collection

### Single Crystal Growth

About 3.24 g of Compound I Phosphate Form I was obtained by salt reaction of Compound I Form I with 3 eq. of phosphoric acid in acetone/water (19/1, v/v) followed by slurry conversion in MeOH with Compound I Phosphate Form I seeds, which can be prepared according to methods described herein. This batch of Compound I Phosphate Form I, as confirmed by XRPD was used as starting material to prepare single crystals.

About 5.0 mg of Compound I Phosphate Form I was added into 2.0 mL of MeOH and stirred magnetically at 50° C. for 20 mm. The solution was filtered into a clean vial and 1 eq. H₃PO₄ was added to inhibit dissociation. The glass vial was covered with pin-hole film and placed in fume hood for slow evaporation at RT. After 3 days, plate-shaped single crystals were generated. The single crystals obtained were used for single crystal X-ray diffraction.

### Instruments and Parameters

Single crystal X-ray diffraction data of Compound I Phosphate Form I was collected at 180 K on a Rigaku XtaLAB Synergy-DW diffractometer, with Cu Kα radiation (l=1.54184 Å). Data reduction and empirical absorption correction were performed using the CrysAlisPro program. The structure was solved by a dual-space algorithm using SHELXT program. All non-hydrogen atoms could be located directly from the difference Fourier maps. Framework hydrogen atoms were placed geometrically and constrained using the riding model to the parent atoms. Final structure refinement was done using the SHELXL program by minimizing the sum of squared deviations of F² using a full-matrix technique.

### Single Crystal X-Ray Diffraction Analysis

Compound I Phosphate Form I crystallizes as monoclinic in P2₁/c space group with formula of C₂₃H₂₉F₂N₅O₁₁P₂. There are one Compound I cation, one phosphate anion and one phosphoric acid molecule in each asymmetric unit, and the unit cell contains four asymmetric units. The phosphate anion is positionally disordered with 0.5 occupancy at two sites. The stoichiometric ratio of Compound I to phosphoric acid is determined to be 1:2.

The crystal structural data are summarized in Table 65. The calculated XRPD pattern from the single crystal structure is consistent with the experimental pattern which is Compound I Phosphate Form.

TABLE 65
Crystal Data and Structure Refinement
for Compound I Phosphate Form I

| | |
|---|---|
| Empirical formula | C₂₃H₂₉F₂N₅O₁₁P₂ |
| Formula weight | 651.45 |
| Temperature/K | 180.00(10) |
| Crystal system | Monoclinic |
| Space group | P2₁/c |
| a/Å | 12.9509(2) |
| b/Å | 8.22450(10) |
| c/Å | 25.4204(3) |
| α/° | 90 |
| β/° | 100.0490(10) |
| γ/° | 90 |
| Volume/Å³ | 2666.11(6) |
| Z | 4 |
| ρ_{calc}g/cm³ | 1.623 |
| μ/mm⁻¹ | 2.258 |
| F(000) | 1352.0 |
| Crystal size/mm³ | 0.15 × 0.04 × 0.02 |
| Radiation | Cu Kα (l = 1.54184 Å) |
| 2θ range for data | 6.932 to 145.142 |
| collection/° | |
| Index ranges | −15 ≤ h ≤ 16, −9 ≤ k ≤ |
| | 10, −31 ≤ l ≤ 30 |
| Reflections collected | 53330 |
| Independent reflections | 5123 [Rᵢₙₜ = 0.0545, |
| | R_{sigma} = 0.0242] |
| Data/restraints/parameters | 5123/24/409 |
| Goodness-of-fit on F² | 1.076 |
| Final R indexes | R₁ = 0.0505, |
| [I >= 2σ (I)] | wR₂ = 0.1334 |
| Final R indexes | R₁ = 0.0545, |
| [all data] | wR₂ = 0.1363 |
| Largest diff. | 0.97/−1.03 |
| peak/hole/e Å⁻³ | |

### Example 6: Salt Screening of Compound I

### Equipment

XRPD patterns were collected with a PANalytical Empyrean diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcellD-Medipix3 PASS (programmable anti-scatter slit), located 240 mm from the sample and Data Collector software v. 7.2b.

Differential Scanning Calorimetry (DSC) data were collected using a TA Instruments Q2000 differential scanning calorimeter. Temperature calibration was performed using NIST-traceable indium metal. The sample was placed into a Tzero aluminum DSC pan, covered with a lid pierced using a needle. The weight was then accurately recorded. A weighed aluminum pan configured as the sample pan was placed on the reference side of the cell. The sample was heated from 20° C. to 300° C. at 10° C./minute. Thermogravimetric Analysis (TGA) data were collected using a TA Instruments Q5000 thermogravimetric analyzer. Temperature calibration was performed using nickel and Alumel™. Each sample was placed in an aluminum pan and inserted into the TG furnace. The furnace was heated under a nitrogen purge. The sample was heated from ambient to 300° C. at 10° C./minute.

### Compound I Malonate Acetonitrile Solvate Form I and Compound I Malonate Form I

### Preparation

Compound I Malonate Form I was prepared by slurrying about 50 mg of Compound I Form I in about 0.5 mL of acetonitrile. Approximately one molar equivalent of malonic acid was added, and the slurry was stirred at ambient temperature for about one day. Solids for Compound I Malonate Acetonitrile Solvate Form I were isolated by centrifugation and characterized by XRPD. The solids were then dried in a vacuum oven at 50° C. for about three days to obtain Compound I Malonate Form I.

### Characterization

The XRPD pattern for Compound I Malonate Acetonitrile Solvate Form I is shown in FIG. 30A and is characterized by peaks at 5.4, 9.8, and 15.4 °2θ, and one or more peaks at 10.1, 12.1, or 17.2 °2θ. Compound I Malonate Acetonitrile Solvate Form I is also characterized by one or more peaks at 14.4, 16.3, or 19.8 °2θ.

The XRPD pattern for Compound I Malonate Form I is shown in FIG. 31A and is characterized by peaks at 5.7, 12.0, and 15.2 °2θ, and one or more peaks at 11.1, 16.1, or 18.0 °2θ. Compound I Malonate Form I is also characterized by one or more peaks at 13.6, 17.0, or 19.3 °2θ.

The DSC thermogram for Compound I Malonate Form I is shown in FIG. 31B. DSC thermogram of Compound I Malonate Form I (FIG. 31B) exhibits three endothermic transitions with onsets at about 32, 168 and 286° C., respectively. The TGA thermogram is shown in FIG. 31C and exhibits a weight loss of about 4.8% during the first endothermic event and a weight loss of about 18.5% during the second endothermic event.

### Compound I Citrate Methanol Solvate Form I, Compound I Citrate Form I, and Citrate Form II Preparation

Compound I Citrate Form I was prepared by slurrying about 50 mg of Compound I Form I in about 0.5 mL of methanol and stirring at ambient temperature for about a day. Approximately one molar equivalent of citric acid was added, and the slurry was stirred at ambient temperature for about three days. Solids for Compound I Citrate Methanol Solvate Form I were isolated by centrifugation and characterized by XRPD. The solids were then dried in a vacuum oven at 50° C. for about one day to obtain Compound I Citrate Form I. The slurry was further stirred at ambient temperature for about two weeks and sonicated for about 10 minutes. The solids were then isolated by centrifugation and dried in a vacuum oven at 50° C. for about one day to obtain Compound I Citrate Form II.

### Characterization

The XRPD pattern for Compound I Citrate Methanol Solvate Form I is shown in FIG. 32A and is characterized by peaks at 4.8, 9.8, and 14.7 °2θ, and one or more peaks at 8.2, 10.5, or 15.6 °2θ. Compound I Citrate Methanol Solvate Form I is also characterized by one or more peaks at 12.4, 15.3, or 17.0 °2θ.

The XRPD pattern for Compound I Citrate Form I is shown in FIG. 33A and is characterized by peaks at 5.3, 10.5, and 16.6 °2θ, and one or more peaks at 8.2, 11.1, or 16.0 °2θ. The Compound I Citrate Form I is also characterized by one or more peaks at 3.7, 11.7, or 13.1 °2θ.

The XRPD pattern for Compound I Citrate Form II is shown in FIG. 34A and is characterized by peaks at 5.2, 10.3, and 14.2 °2θ, and one or more peaks at 7.9, 15.8, or 16.3 °2θ. The Compound I Citrate Form II is also characterized by one or more peaks at 3.6, 8.2, or 19.8 °2θ.

The DSC thermogram for Compound I Citrate Form II is shown in FIG. 34B. DSC thermogram of Compound I Citrate Form II (FIG. 34B) exhibits four endothermic transitions with onsets at about 47, 141, 169 and 286° C. The TGA thermogram is shown in FIG. 34C and exhibits two weight losses of about 3.1% and 26.5%, respectively.

### Compound I Oxalate Acetonitrile Solvate Form I and Compound I Oxalate Form I

### Preparation

Compound I Oxalate Form I was prepared by slurrying about 50 mg of Compound I Form I in about 0.5 mL of acetonitrile. Approximately one molar equivalent of oxalic acid was added, and the slurry was stirred at ambient temperature for about a day. Solids for Compound I Oxalate Acetonitrile Solvate Form I were isolated by centrifugation and characterized by XRPD. The solids were then dried in a vacuum oven at about 50° C. for about three days to obtain Compound I oxalate Form I.

### Characterization

The XRPD pattern for Compound I Oxalate Acetonitrile Solvate Form I is shown in FIG. 35A and is characterized by peaks at 5.1, 15.4, and 16.6 °2θ, and one or more peaks at 11.0, 14.2, or 21.3 °2θ. Compound I Oxalate Acetonitrile Solvate Form I is also characterized by one or more peaks at 20.6, 24.0 or 27.1 °2θ.

The XRPD pattern for Compound I Oxalate Form I is shown in FIG. 36A and is characterized by peaks at 5.6, 12.9, and 20.6 °2θ, and one or more peaks at 11.0, 14.0, or 21.3 °2θ. Compound I Oxalate Form I is also characterized by one or more peaks at 16.6, 18.2, or 27.2 °2θ.

### Alternative Preparations of Compound I Phosphate Form I

Compound I Form 1(20 g, 1.0 eq.) and methanol (600 mL, 30 V) were charged in a flask at 20° C. H₃PO₄ (10.13 g, 2.0 eq. 85%) was added to the flask at 20° C. The mixture was stirred for 2 h at 20° C. Compound I Phosphate Form I seeds (1 g, 0.0018 eq), which can be prepared as described herein, was added, and the mixture stirred for 2 days at 20° C. The mixture was then filtered, and the filter cake was washed with MeOH (200 mL) and dried to obtain Compound I Phosphate Form I.

Compound I Phosphate Form I was prepared by slurrying about 5 grams of Compound I Form I in about 130 mL of methanol. Approximately two molar equivalents of phosphoric acid (85%) were added, and the slurry was stirred at ambient temperature. Around 60 mg of Compound I Phosphate Form I seeds, which were prepared as described herein, were charged. The slurry was heated to about 40° C. and agitated for about one day. The slurry was cooled to about 20° C. and agitated for about one day. The slurry was then filtered and washed with around 26 mL methanol. The solids were then dried in a vacuum oven at 50° C. for about one day, to obtain Compound I Phosphate Form I.

The XRPD pattern for Compound I Phosphate Form I is shown in FIG. 15A and is characterized by peaks at 9.1, 14.6, and 18.2 °2θ, and one or more peaks at 7.1, 14.3, or 17.6 °2θ. Compound I Phosphate Form I is also characterized by one or more peaks at 16.9, 21.1, or 24.5 °2θ.

The DSC thermogram for Compound I Phosphate Form I is shown in FIG. 15B and exhibits one endothermic transition with onset at about 270° C. The TGA thermogram is shown in FIG. 15C, and exhibits a weight loss of about 1.2% prior to around 240° C. About 9.2% weight loss is observed during the endothermic event.

Compound I Phosphate Form I can also be prepared according to the following procedure.

Compound I Phosphate Form I was prepared by charging Compound I Form I (1.0 X), dichloromethane (about 31.8 X, 24 V) and methanol (about 4.8 X, 6 V) to a reactor. The contents of the reactor were agitated at about 22° C. until all solids dissolved. The contents of the reactor were then distilled under vacuum to about 18 V, with maximum jacket temperature around 40° C. The reactor contents were adjusted to around 22° C. About 2 equivalents of phosphoric acid, technical grade, 85% (about 0.51×) were charged to the reactor and agitated for around 15 minutes at around 22° C. Compound I Phosphate Form I seeds (around 0.01 X), which were prepared as described herein, were then charged to the reactor. The contents of the reactor were adjusted to around 35° C. The contents were agitated at around 35° C. until the reaction was deemed complete by XRPD (around 24 h expected). The reactor contents were adjusted to around 22° C. The contents of the reactor were filtered and rinsed forward with methanol (around 4.0 X, 5 V). The solids were dried under vacuum at about 50° C. and with a N₂ sweep for about 1 day to obtain Compound I Phosphate Form I.

### Alternative Preparation of Compound I Phosphate Form II

Compound I Phosphate Form II was also prepared by dispersing about 60 mg of Compound I Form I in about 2 mL ethanol. Then about 2.5 eq. of phosphoric acid were added and the contents were stirred at room temperature for about 1 day. The solids were collected by filtration and characterized by XRPD to obtain Compound I Phosphate Form II.

### Alternative Preparation of Compound I Fumarate Form I

Compound I Fumarate Form I was prepared by slurrying about 200 mg of Compound I Form I in about 2 mL of methanol. Approximately two molar equivalents of fumaric acid were added. The slurry was stirred at ambient temperature for about one day. Around 1.5 mL of methanol were added, and the slurry was sonicated for about 10 minutes. The slurry was agitated for a few hours and filtered under vacuum. Around 0.5 mL of methanol were used to rinse forward. The solids were dried in a vacuum oven at 50° C. for about one day to obtain Compound I Fumarate Form I.

The XRPD pattern for Compound I Fumarate Form I is shown in FIG. 16A and is characterized by peaks at 5.5, 7.0, and 19.4 °2θ, and one or more peaks at 7.7, 10.9, or 16.4 °2θ. Compound I Fumarate Form I is also characterized by one or more peaks at 14.0, 18.1, or 21.3 °2θ.

The DSC thermogram for Compound I Fumarate Form I is shown in FIG. 16B and exhibits two endothermic transitions with onsets at about 188° C. and 261° C.

The TGA thermogram for Compound I Fumarate Form I is shown in FIG. 16C and exhibits negligible weight loss before 100° C., around 2.6% weight loss between 10° and 188° C., and around 11.8% weight loss between 188° C. and 279° C.

### Compound I Fumarate Form II

About 300 mg of Compound I Form I in about 5 mL of methanol was slurried. Approximately two molar equivalents of fumaric acid were added and the slurry was stirred for about one day. A small amount of Compound I Fumarate Form I seeds (Preparation: About 50 mg of Compound I Form I was slurried in about 0.4 mL of methanol. Approximately one molar equivalents of fumaric acid were added and the slurry was stirred for about one day and diluted with about 0.4 mL methanol. The slurry was centrifuge-filtered after stirring for about 11 days and dried at 50° C. for about 1 day) were added and the slurry was stirred at ambient temperature for about three days, diluted with about 1 mL methanol, filtered under vacuum and dried at about 50° C. for about one day.

Compound I Fumarate Form II was prepared by slurrying about 50 mg of Compound I Form I in about 1 mL of methanol/water solvent mixture (1/1, v/v). Approximately two molar equivalents of fumaric acid were added. A small amount of Compound I Fumarate Form I seeds, which were prepared as described above, were added, and the slurry was stirred at ambient temperature for about one day. A slurry sample was centrifuge filtered, and the solids were analyzed by XRPD to obtain Compound I Fumarate Form II. The solids were then dried in a vacuum oven at 50° C. for about one day to obtain Compound I Fumarate Form II.

The XRPD pattern for Compound I Fumarate Form II is shown in FIG. 37A and is characterized by peaks at 4.2, 8.5, and 12.7 °2θ, and one or more peaks at 8.8, 14.2, or 18.6 °2θ. Compound I Fumarate Form II is also characterized by one or more peaks at 10.2, 17.6, or 21.3 °2θ.

The DSC thermogram for Compound I Fumarate Form II is shown in FIG. 37B and exhibits four endothermic transitions with onsets at about 23° C., 190° C., 218° C. and 250° C.

1. A crystalline form of Compound I: 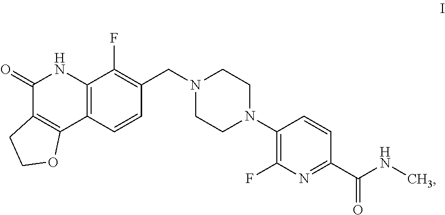 [SMILES: CNC(=O)c1ccc(N2CCN(Cc3ccc4c5c(c(=O)[nH]c4c3F)CCO5)CC2)c(F)n1]
(Compound I Form I), wherein Compound I Form I is characterized by an X-ray powder diffractogram comprising peaks (±0.2°) at 8.1, 9.4, and 19.7 °2θ as determined on a diffractometer using Cu-Kα radiation.

2. The crystalline form of claim 1, further characterized by:
i) one or more peaks at (±0.2°) at 4, 12.2, or 18.8 °2θ;
ii) a diffractogram substantially as shown in FIG. 1A;
iii) a differential scanning calorimetry (DSC) curve comprising an endotherm at about 285° C. (onset temperature);
iv) a differential scanning calorimetry (DSC) curve substantially as shown in FIG. 1B;
v) thermogravimetric analysis (TGA) showing a weight loss of about 0.5 wt % up to about 65° C.;
vi) thermogravimetric analysis (TGA) comprising a thermogram substantially as shown in FIG. 1B; or
vii) a dynamic vapor sorption (DVS) curve showing about 1.04% water uptake at 80% relative humidity (RH) or about 1.19% water uptake at 90% RH; or
viii) a DVS curve substantially as shown in FIG. 1C.

3. A crystalline form of Compound I: 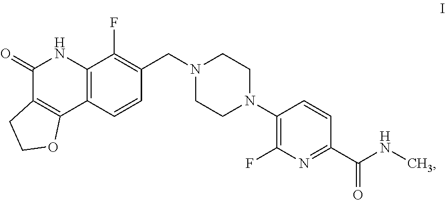 [SMILES: CNC(=O)c1ccc(N2CCN(Cc3ccc4c5c(c(=O)[nH]c4c3F)CCO5)CC2)c(F)n1]
(Compound I Form II), wherein Compound I Form II is characterized by an X-ray powder diffractogram comprising peaks (±0.2°) at 6.4, 9.1, and 18.7 °2θ as determined on a diffractometer using Cu-Kα radiation.

4. The crystalline form of claim 3, further characterized by:
i) one or more peaks at (±0.2°) at 9.7, 14.1, or 19.5 °2θ;
ii) a diffractogram substantially as shown in FIG. 2A;
iii) a differential scanning calorimetry (DSC) curve comprising an endotherm at about 159° C. (onset temperature) and an endotherm at about 287° C. (onset temperature);
iv) a differential scanning calorimetry (DSC) curve substantially as shown in FIG. 2B;
v) thermogravimetric analysis (TGA) showing a weight loss of about 7.6 wt % up to about 185° C.; or
vi) thermogravimetric analysis (TGA) comprising a thermogram substantially as shown in FIG. 2B.

5. A crystalline form of Compound I: 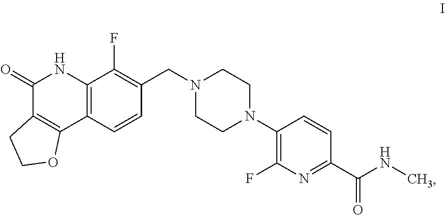 [SMILES: CNC(=O)c1ccc(N2CCN(Cc3ccc4c5c(c(=O)[nH]c4c3F)CCO5)CC2)c(F)n1]
(Compound I Form III), wherein Compound I Form III is characterized by an X-ray powder diffractogram comprising peaks (±0.2°) at 4.7, 9.5, and 12.6 °2θ as determined on a diffractometer using Cu-Kα radiation.

6. The crystalline form of claim 5, further characterized by:
i) one or more peaks at (±0.2°) at 7.5, 11.7, or 18.3 °2θ;
ii) a diffractogram substantially as shown in FIG. 3A;
iii) a differential scanning calorimetry (DSC) curve comprising an endotherm at about 268° C. (onset temperature) and an endotherm at about 281° C. (onset temperature);
iv) a differential scanning calorimetry (DSC) curve substantially as shown in FIG. 3B;
v) thermogravimetric analysis (TGA) showing a weight loss of about 1.2 wt % up to about 290° C.;
vi) thermogravimetric analysis (TGA) comprising a thermogram substantially as shown in FIG. 3B;
vii) a dynamic vapor sorption (DVS) curve showing about 0.77% water uptake at 80% RH or about 1.13% at 90% RH; or
viii) a DVS curve substantially as shown in FIG. 3C.

7-76. (canceled)

77. A pharmaceutical composition comprising the crystalline form of claim 1, and a pharmaceutically acceptable excipient.

78. A method of treating cancer in a subject in need thereof, the method comprising administering the crystalline form of claim 1.

79. The method of claim 78, wherein the cancer is breast cancer, ovarian cancer, pancreatic cancer, prostate cancer, a hematological cancer, gastrointestinal cancer, or lung cancer.

80. A method of treating a cancer comprising a BRCA1 and/or a BRCA2 mutation in a subject in need thereof, the method comprising administering the crystalline form of claim 1.

81. The method of claim 80, wherein the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.
